(19) United States
(12) Patent Application Publication
(10) Pub. No.: US 2018/0117151 A1
(43) Pub. Date: May 3, 2018

(54) PULMONARY INTRAVASCULAR NON-CLASSICAL MONOCYTES MEDIATE LUNG TRANSPLANT ISCHEMIA-REPERFUSION INJURY

(71) Applicant: Northwestern University, Evanston, IL (US)

(72) Inventors: Ankit Bharat, Chicago, IL (US); G.R. Scott Budinger, Evanston, IL (US)

(73) Assignee: Northwestern University, Evanston, IL (US)

(21) Appl. No.: 15/799,474

(22) Filed: Oct. 31, 2017

Related U.S. Application Data
(60) Provisional application No. 62/414,985, filed on Oct. 31, 2016.

(51) Int. Cl. A61K 39/395; C07F 9/40; A61K 9/127; C07C 229/16

(52) U.S. Cl. CPC A61K 39/39558; C07C 229/16; A61K 9/127; C07F 9/4037

(57) ABSTRACT

Disclosed are methods and compositions which may be utilized to inhibit and/or deplete non-classical monocytes during a transplantation procedure. The disclosed methods and compositions may be utilized in order to reduce damage to organs and tissues during a transplantation procedure and/or to improve the success rate of the transplantation procedure.

### CROSS-REFERENCE TO RELATED PATENT APPLICATIONS

The present application claims the benefit of priority under 35 U.S.C. § 119(e) to U.S. Provisional Patent Application No. 62/414,985, filed on Oct. 31, 2016, the content of which is incorporated herein by reference in its entirety.

### BACKGROUND

The present invention relates to methods and compositions for improving a transplantation procedure. The disclosed methods and compositions typically utilize or include pharmaceutical agents that inhibit and/or deplete non-classical monocytes during the transplantation procedure.

### SUMMARY

Disclosed are methods and compositions which may be utilized to inhibit and/or deplete non-classical monocytes during a transplantation procedure. The disclosed methods and compositions may be utilized in order to reduce damage to organs and tissues during a transplantation procedure and/or to improve the success rate of the transplantation procedure.

In some embodiments, the disclosed methods include treating a donor for a transplantation procedure prior to harvesting an allograft from the donor with a pharmaceutical agent that inhibits the activity of non-classical monocytes. In other embodiments, the disclosed methods include treating an allograft that has been removed from a donor and prior to a transplantation procedure with a pharmaceutical agent that inhibits the activity of non-classical monocytes and/or an agent that depletes the allograft of non-classical monocytes. The disclosed methods also include methods for preserving an allograft after harvest by storing the allograft in a preservation solution comprising an agent inhibits the activity of non-classical monocytes and/or a chelator of divalent cations.

The disclosed compositions include preservation solutions for allografts. The preservation solutions disclosed herein may include at least one of (i) an agent that inhibits the activity of non-classical monocytes, and/or (ii) a chelator of divalent cations. The disclosed compositions also include an allograft stored in a preservation solution.

### BRIEF DESCRIPTION OF THE FIGURES

FIG. 1A, FIG. 1B, FIG. 1C, FIG. 1D, and FIG. 1E. Donor intravenous clodronate-liposome treatment ameliorates primary graft dysfunction following transplantation. (A) Experimental design. (B) Allograft histology. Representative histology of the heart-lung blocks of control PBS-liposome and intravenous (IV) Clodronate-liposome (clo-lip) treated donors at 24 hours after reperfusion. Hematoxylin and eosin staining. Inset scale bars represent 50 μm. (C) Allograft function measured by PaO₂ on 100% FiO₂ as a marker of lung injury at 24 hours post-transplant, *p=0.01. (D) Allograft edema measured by wet-to-dry ratio of allograft at 4 and 24 hours after reperfusion, *p<0.001, n=5 per group. (E) Allograft vascular permeability measured by Evans Blue Dye extravasation leak test of allograft at 4 and 24 hours after reperfusion, *p<0.01. n=5 per group. Unpaired student's t-test was used to compare means.

FIG. 2A, FIG. 2C, and FIG. 2C. Intravenous clodronate-liposomes selectively depletes nonclassical monocytes in donor lungs. (A) Experimental design. (B) Flow cytometry plots showing effects of monocyte depletion strategies on classical (CM), non-classical (NCM), and CD11b⁺ dendritic cells (DC) in wild type mice (Full gating strategy shown in FIG. 10). (C) Effects of Clodronate-liposome (clo-lip) and anti-CCR2 on number of monocytes and relative composition of lung monocytes at 24 hours after treatment. *p<0.01, n=5 per group. (C) Data are representative of 5 experiments. Unpaired student's t-test with Holm-Sidak correction for multiple comparisons was used to compare means.

FIG. 3A, FIG. 3B, FIG. 3C, FIG. 3D, FIG. 3E, FIG. 3F, and FIG. 3G. Influx of recipient neutrophils into the allograft is abrogated by depletion of donor NCM. (A) Experimental design (B-G). Intravital two photon imaging at 2 hours starting at t=0 through t=30 minutes following reperfusion. Representative still images of control PBS-lip treated donor allograft immediately after reperfusion are shown, Green: LysM⁺, Red: Q655vascular blood vessels. Unpaired student's t-test was used to compare means. (H) Experimental design and result of differential monocyte depletion strategies in donors and recipients. Combinations of donor and recipient treatments were used to selectively deplete the different monocyte populations. The allograft was harvested at 24 hours following transplantation and neutrophil influx determined using flow cytometry, *p=0.001 compared to Group I, all other comparisons to Group I are not significant, n=6 per group. Unpaired student's t-test with Holm-Sidak correction for multiple comparisons was used to compare means.

FIG. 4A, FIG. 4B, FIG. 4C, and FIG. 4D. Pulmonary intravascular nonclassical monocytes (NCM) are dependent on CX3CR1 to recruit neutrophils and reconstitution of depleted NCM restores neutrophil influx in NR4A1 deficient mice. (A) Experimental design for CX3CR1 knockout transplants. (B) Representative flow plots and effects of CX3CR1 deletion on monocyte populations in donor lungs and post-transplant neutrophil infiltration of the allograft. *p=0.01, n=6 per group. Unpaired student's t-test was used to compare means. (C) Experimental design for NR4A1 knockout transplants. (D) Representative flow plots and effects of NR4A1 deletion NCM reconstitution on monocyte populations in donor lungs and post-transplant neutrophil infiltration. *p=0.01, n=5 per group. Unpaired student's t-test with Holm-Sidak correction for multiple comparisons was used to compare means. CM: Classical Monocyte, NCM: Nonclassical Monocyte, DC: Dendritic cell.

FIG. 5A, FIG. 5B, FIG. 5C, FIG. 5D, and FIG. 5E. Compartmentalization of pulmonary nonclassical and classical monocytes and their response to intratracheal lipopolysaccharide. (A) Two-photon imaging of Cx3cr1^{gfp/+} lungs. Green: Cx3cr1⁺, Red: Q655vascular blood vessels, Blue: Second harmonic generation collagen, Yellow: Autofluorescent alveolar macrophages. Filled arrow: Extravascular cell. Open arrow: Intravascular cell. (B) Morphology of the lung myeloid cell populations in the donor lung using immunogold electron microscopy in the Cx3cr1^{gfp/+} reporter mouse. Left panel: Immuno-EM of fixed Cx3cr1^{gfp/+} lung with white circles highlighting gold nanoparticles staining for GFP. Right panels: EM micrographs of post-sort cells from WT B6 mouse lung. (C) Immuno-EM of Cx3cr1^{gfp/+} donor lungs at 4-hours after reperfusion with white circles highlighting gold nanoparticles staining for GFP. Left and middle panels: Non-classical monocyte bound to the endothelium with areas of exposed, thickened basement membrane and endothelial cell blebbing. Right panels: Neutrophil bound to the endothelium in the vicinity of a blebbing endothelial cell. (D) Representative compartmental staining of non-classical monocytes and alveolar macrophages. As negative control, FMO+1 staining is shown. IV:intravenous, IT:Intratracheal, FMO: Fluorescence minus one control. (E) Experimental design and representative flow diagrams of intravenous and intratracheal anti-CD45 staining in LPS-treated and control mice along with neutrophil infiltration into the lungs with and without intravenous clo-lip pre-treatment, unpaired student's t-test, not significant. n=5 per group.

FIG. 6A, FIG. 6B, FIG. 6C, and FIG. 6D. Transcriptional profiling of murine post-transplant donor-derived nonclassical monocytes. (A) Experimental plan to isolate and sort nonclassical monocytes (NCM) from donor naïve, +2 hours, and +24 hours post-transplant lung. (B) Sorting strategy with representative flow plots from each time point. (C) Principle components analysis of samples. (D) Gene ontology process enrichment analysis using K-means clustering. Toll-like receptor and NFkB signaling pathway genes which were differentially expressed by cutoff of adjusted p<0.05 by pairwise comparison between time points with the normalized counts of genes of interest depicted. *p<0.04 by one-way ANOVA, n=4 per group. Heatmap scale bars represent log₂ scale (data not shown).

FIG. 7A, FIG. 7B, FIG. 7C, FIG. 7D, and FIG. 7E. NCM are dependent on MyD88/TRIF signaling to produce CXCL2. (A) Experimental design. (B) Effects of reconstitution of non-classical monocyte (NCM)-depleted donor lungs with either wild type (WT) or Myd88/Trif^{-/-} NCM. *p<0.01, n=5 per group. (C) Cxcl2 transcript expression level in donor-derived NCM measured by qPCR at two hours post-transplant. As control, the baseline expression of Cxcl2 in wild type pulmonary NCM is shown, *p=0.01. (D) Effect of NCM depletion on CXCL2 cytokine levels in allograft circulation when NCM are depleted. As control, the CXCL2 levels in left pulmonary vein blood of wild type mice is shown. *p<0.01, n=5 per group. Unpaired student's t-test with Holm-Sidak correction for multiple comparisons was used to compare means. (E) Effect of CXCL2 blockade on neutrophil recruitment in the recipient at 24 hours after transplant. *p=0.001, n=5 per group. Unpaired student's t-test was used to compare means.

FIG. 8A, FIG. 8B, and FIG. 8C. Myeloid cell populations in human donor lungs and immediate post-reperfusion changes. (A) Representative gating strategy of human lungs flushed and utilized in clinical transplantation. After excluding doublets and dead cells, and including only CD45⁺ cells, neutrophils were identified as CD15⁺CD16⁺SSC^{high}. After gating out CD15⁺ events, an HLA⁻DR⁺CD11b⁺ gate was used to identify monocytes and macrophages. AM were identified as CD15⁻HLA⁻DR⁺CD11b⁺CD169⁺CD206⁺. After gating out AM, NCM were identified as CD16⁺⁺CD14^{dim}, IntM as CD16⁺CD14⁺, and CM as CD14⁺CD16⁻. (B) Changes in nonclassical monocytes and neutrophils at 90 minutes after reperfusion. Data expressed as cell count per alveolar macrophage to standardize across patients. Biopsies were taken serially from the same location in the lung. *p=0.02 by paired student's t-test, n=8. Immunofluoresence microscopy of (C) pre-reperfusion and (D) post-reperfusion human lung samples depicting endothelial-bound intravascular CD16⁺CD14^{dim} NCM (Filled white arrow) in contrast with CD16⁻ CD14^{high} CM (Open arrow) and CD16⁺CD14⁺ neutrophils (Filled white chevron). Scale bar represents 10 μm. Green: CD31, Blue: DAPI, Red: CD16, Yellow: CD14. NCM: Nonclassical Monocyte, IntM: Intermediate Monocyte, CM: Classical Monocyte, AM: Alveolar Macrophage.

FIG. 9. Cytokine analysis of the lung allograft. Fold-increase of broncho-alveolar lavage (BAL) cytokines in PBS-lip versus Clo-lip treated allografts (n=5 per group).

FIG. 10. Representative gating strategy to evaluate the myeloid cell populations in the lungs. After excluding doublets and dead cells, and including only CD45⁺ cells, neutrophils were identified as Ly6G⁺CD11b⁺CD24⁺, while NK cells (NKC) were identified as NK1.1⁺CD24⁻CD11b^{low/int}. Eosinophils (Eos) were identified as Ly6G^{low}NK1.1⁻SiglecF⁺CD64⁻. After excluding eosinophils, neutrophils, and NK cells, CD103⁺ DC (dendritic cells) were identified as being CD11b⁻CD64⁻CD24⁺CD11c⁺ and alveolar macrophages (AM) as being CD11b⁻CD64⁺SiglecF⁺. After exclusion of CD11b⁻ cells, interstitial macrophages (IM) were identified as being CD64⁺Ly6C^{+/-} and the remaining CD64⁻ cells were either Ly6C^{high}MHCII^{+/-} classical monocytes (CMo), Ly6C^{+/-}MHCII⁻ non-classical monocytes (NCMo), or Ly6C^{low}MHCII⁺ CD11b⁺ DC.

FIG. 11. Perfusion of the heart-lung block. There are no intravascular red blood cells visualized in the perfused whole heart-lung-block: Compared with non-perfused block (left side), the red blood cells (black arrow) were flushed thoroughly in the perfused whole lung block (right side) on both gross and histological evaluation (×200).

FIG. 12A and FIG. 12B. Effects of monocyte depletion strategies on the donor. (A) Effects of clo-lip treatment on circulating monocytes in the blood and monocytes in the spleen. *p<0.005, by student's t-test with Holm-Sidak adjustment for multiple comparisons, n=3 per group. (B) Effects of clo-lip and anti-CCR2 treatment on non-monocyte lung resident myeloid populations. No comparison by one way ANOVA with Tukey's correction for multiple comparisons met the significance threshold of p<0.05. n=3-6 per group.

FIG. 13. Effects of donor clo-lip treatment on non-neutrophil myeloid cell types. All comparisons were non-significant by unpaired student's t-test with Holm-Sidak adjustment for multiple comparisons. n=5 per group.

FIG. 14. Donor clo-lip treatment abrogates neutrophil influx after syngeneic lung transplant. (A) Experimental design. (B) Effects of donor clo-lip pre-treatment on post-transplant neutrophil infiltration at 24 hours. *p=0.001, n=5 per group.

FIG. 15A, FIG. 15B, FIG. 15C, FIG. 15D, FIG. 15E, and FIG. 15F. Effects of CX3CR1 deletion on nonmonocyte cell populations in the lung and blood and on the posttransplant allograft. Effects of CX3CR1 homozygous deletion versus heterozygous control on the (A) non-monocyte myeloid populations in the lung and (B) myeloid populations in the blood. *p=0.02, n=5 per group. (C) 24 hour post-transplant heart-lung block with inset representing 10× magnification. (D) Effects of CX3CR1 homozygous deletion versus heterozygous control on 24 hour post-transplant allograft BAL cytokines. n=5 per group. (E) PaO2 on 100% FiO2 of CX3CR1 deficient versus wild type donors. *p=0.01, n=5 per group. (F) Effects of anti-CX3CR1 blockade on neutrophil infiltration into the allograft at 24 hours post-transplant. *p=0.002, n=5 per group. All comparisons were made using an unpaired student's t-test using a Holm-Sidak adjustment for multiple comparisons where appropriate.

FIG. 16A, FIG. 16B, FIG. 16C, and FIG. 16D. Effects of NR4A1 deletion on non-monocyte cell populations in the lung and blood and on the posttransplant allograft. Effects of NR4A1 homozygous deletion versus wild type (WT) control on the (A) non-monocyte myeloid populations in the lung and (B) myeloid populations in the blood. *p=0.0001, n=5 per group. (C) 24 hour post-transplant heart-lung block with inset representing 10× magnification. (D) Effects of NR4A1 deletion and reconstitution of NCM in Nr4a1−/− on non-monocyte cell populations in the donor lung. No comparison by one-way ANOVA with Tukey's correction for multiple comparisons met the significance threshold of p<0.05.

FIG. 17. All neutrophils were of recipient origin after 24 hours reperfusion in allograft. Using the orthotopic left lung transplant model from C57BL/6 (CD45.2) to Balb/c (CD45.1), Multicolor flow cytometry analysis showed all the neutrophils were recipient derived in allograft lung at 24 hours after reperfusion.

FIG. 18. Representative gating strategy of GFP-expressing cells in lungs of Cx3cr1^{gfp/+} and Cx3cr1^{gfp/gfp} mice. After exclusion of doublets and dead cells and including only CD45⁺ cells for analysis, the GFP⁺ population was found to include NK1.1⁺SSC^{low}CD11b^{int} NK cells, Ly6G⁻NK1.1⁻CD11b⁺CD64⁺ interstitial macrophages or monocyte-derived cells (IM/MoDC), and Ly6C^{high}MHCII^{+/-} classical monocytes (CMo), Ly6C^{low}MHCII⁻ non-classical monocytes (NCMo), and Ly6C^{low}MHCII⁺ CD11b+ dendritic cells (DC).

FIG. 19. NCMs do not differentiate upon lipopolysaccharide challenge. Sorted CD45.1 WT NCM were used to reconstitute CD45.2 Nr4a1−/− and 24 hours later were challenged with intratracheal lipopolysaccharide. At 24 hours post-LPS challenge, lungs were harvested for flow cytometry. Representative flow plots and histograms are shown (n=3).

FIG. 20. Immunofluorescence microscopy of human lung biopsy from a patient experiencing PGD. CD16+CD14dim NCM (filled white arrows) in the vicinity of clusters of neutrophils (filled white chevrons) and classical monocytes (open arrows) in lung biopsy from a patient experiencing early severe primary graft dysfunction. Green: CD31+, Blue: DAPI+, Red: CD16+, Yellow: CD14+. Scale bar represents 10 μm.

FIG. 21A, FIG. 21B, and FIG. 21C. Schematic to illustrate the role of pulmonary intravascular NCMs in mediating neutrophil infiltration and lung allograft injury. (A) At the time of procurement, donor lungs are perfused with a preservative solution to flush all unbound circulating intravascular cells but is unable to remove the NCM bound to the endothelium. Hence, pulmonary intravascular monocytes are retained in the donor lungs. Additionally, interstitial classical monocytes are retained in the donor lungs. (B) Following reperfusion of the lung allograft, the donor-derived pulmonary intravascular NCM are activated through a Toll-like receptor signaling pathway dependent on MyD88 or TRIF which leads to the production of MIP-2, a key neutrophil chemoattractant, leading to neutrophil influx into the allograft and resulting in primary graft dysfunction. (C) CX3CR1 blockade, MIP-2 neutralization, or non-classical monocyte depletion represent strategies to ameliorate primary graft dysfunction.

### DETAILED DESCRIPTION

The present invention is described herein using several definitions, as set forth below and throughout the application.

Unless otherwise specified or indicated by context, the terms “a”, “an”, and “the” mean “one or more.” For example, “a pharmaceutical agent” should be interpreted to mean “one or more pharmaceutical agents.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

Methods and Compositions for Improving Transplantation Procedures

The methods and compositions disclosed herein may be utilized to inhibit and/or deplete non-classical monocytes during a transplantation procedure. In particular, the disclosed methods and compositions may be utilized in order to reduce damage to organs and tissues during a transplantation procedure and/or to improve the success rate of the transplantation procedure.

In some embodiments of the disclosed methods, the methods include treating a donor prior to harvesting an allograft from the donor with a pharmaceutical agent that inhibits the activity of non-classical monocytes. Suitable agents for the disclosed methods may include agents that induce apoptosis of the non-classical monocytes (e.g., liposome-loaded agents such as clodronate). Suitable agents for the disclosed methods also may include cytotoxic antibodies that are targeted to non-classical monocytes (e.g., a cytotoxic antibody that is targeted to the cell surface protein 6-sulfo LacNAc (SLAN) on non-classical monocytes). Suitable agents for the disclosed methods also may include antibodies that suppress the function of non-classical monocytes (e.g., an antibody that binds to the cell surface receptor CS3CR1). These disclosed methods further may include harvesting the allograft and subsequently storing the allograft in a preservation solution after having treated the donor with the pharmaceutical agent that inhibits the activity of non-classical monocytes. The subsequently used preservation solution may include the aforementioned pharmaceutical agent and/or a chelator of divalent cations (e.g., EDTA and EGTA). Suitable allografts for these methods may include an organ or part of an organ selected from the group consisting of, but not limited to, lung, kidney, pancreas, intestine, and liver.

In other embodiments of the disclosed methods, the methods include treating an allograft prior to transplantation with an agent that inhibits the activity of non-classical monocytes and/or an agent that depletes the allograft of non-classical monocytes. Suitable agents for the disclosed methods may include an agent that induces apoptosis of the non-classical monocytes, an agent that is a cytotoxic antibody that is targeted to the non-classical monocytes (e.g., a cytotoxic antibody is targeted to the cell surface protein 6-sulfo LacNAc (SLAN) on the non-classical monocytes, and/or an antibody that suppresses the function of non-classical monocytes (e.g., an antibody that binds to the cell surface receptor CS3CR1). Suitable agents also may include agents that facilitate dissociation of non-classical monocytes from the allograft. In some embodiments, the agents may include a chelator of divalent cations (e.g., EDTA and EGTA). Suitable allografts for these methods may include an organ or part of an organ selected from the group consisting of, but not limited to, lung, kidney, pancreas, intestine, and liver.

In other embodiments of the disclosed methods, the methods include preserving an allograft after harvest. These disclosed methods typically include storing the allograft in a preservation solution comprising an agent inhibits the activity of non-classical monocytes as aforementioned herein, and/or a chelator of divalent cations as aforementioned herein. Suitable allografts for these methods may include an organ or part of an organ selected from the group consisting of, but not limited to, lung, kidney, pancreas, intestine, and liver.

In some embodiments of the disclosed compositions, the disclosed compositions include preservation solutions for harvested allografts. The preservation solutions typically include at least one of (i) an agent inhibits the activity of non-classical monocytes as aforementioned herein; and/or (ii) a chelator of divalent cations as aforementioned herein. The compositions disclosed herein may include (a) a harvested allograft in the aforementioned preservation solution. Suitable allografts for these compositions may include an organ or part of an organ selected from the group consisting of, but not limited to, lung, kidney, pancreas, intestine, and liver.

### ILLUSTRATIVE EMBODIMENTS

The following embodiments are illustrative and are not intended to limit the scope of the claimed subject matter.

Embodiment 1. A method comprising treating a donor prior to harvesting an allograft from the donor with a pharmaceutical agent that inhibits the activity of non-classical monocytes.

Embodiment 2. The method of embodiment 1, wherein the agent induces apoptosis of the non-classical monocytes.

Embodiment 3. The method of embodiment 2, wherein the agent is loaded in liposomes.

Embodiment 4. The method of any of the foregoing embodiments, wherein the agent is clodronate.

Embodiment 5. The method of embodiment 1, wherein the agent is a cytotoxic antibody that is targeted to the non-classical monocytes.

Embodiment 6. The method of embodiment 5, wherein the cytotoxic antibody is targeted to the cell surface protein 6-sulfo LacNAc (SLAN) on the non-classical monocytes.

Embodiment 7. The method of embodiment 1, wherein the agent is an antibody that suppresses the function of non-classical monocytes.

Embodiment 8. The method of embodiment 7, wherein the antibody binds to the cell surface receptor CS3CR1.

Embodiment 9. The method of any of the foregoing embodiments further comprising harvesting the allograft and storing the allograft in a preservation solution.

Embodiment 10. The method of embodiment 9, wherein the preservation solution comprises the pharmaceutical agent of embodiment 1.

Embodiment 11. The method of embodiment 9 or 10, wherein the preservation solution comprises a chelator of divalent cations.

Embodiment 12. The method of embodiment 11, wherein the chelator is selected from EDTA and EGTA.

Embodiment 13. The method of any of the foregoing embodiments, wherein the allograft comprises an organ or part of an organ selected from the group consisting of lung, kidney, pancreas, intestine, and liver.

Embodiment 14. A method comprising treating an allograft prior to transplantation with an agent that inhibits the activity of non-classical monocytes and/or an agent that depletes the allograft of non-classical monocytes.

Embodiment 15. The method of embodiment 14, wherein the agent induces apoptosis of the non-classical monocytes.

Embodiment 16. The method of embodiment 15, wherein the agent is a cytotoxic antibody that is targeted to the non-classical monocytes.

Embodiment 17. The method of embodiment 16, wherein the cytotoxic antibody is targeted to the cell surface protein 6-sulfo LacNAc (SLAN) on the non-classical monocytes.

Embodiment 18. The method of embodiment 14, wherein the agent is an antibody that suppresses the function of non-classical monocytes.

Embodiment 19. The method of embodiment 18, wherein the antibody binds to the cell surface receptor CS3CR1.

Embodiment 20. The method of embodiment 14, wherein the agent is a chelator of divalent cations.

Embodiment 21. The method of embodiment 20, wherein the chelator is selected from EDTA and EGTA.

Embodiment 22. The method of any of embodiments 14-21, wherein the allograft comprises an organ or part of an organ selected from the group consisting of lung, kidney, pancreas, intestine, and liver.

Embodiment 23. A method for preserving an allograft after harvest, the method comprising storing the allograft in a preservation solution comprising an agent inhibits the activity of non-classical monocytes and/or a chelator of divalent cations.

Embodiment 24. The method of embodiment 23, wherein the agent induces apoptosis of non-classical monocytes.

Embodiment 25. The method of embodiment 23, wherein the agent is a cytotoxic antibody that is targeted to the non-classical monocytes.

Embodiment 26. The method of embodiment 25, wherein the cytotoxic antibody is targeted to the cell surface protein 6-sulfo LacNAc (SLAN) on the non-classical monocytes.

Embodiment 27. The method of embodiment 23, wherein the agent is an antibody that suppresses the function of non-classical monocytes.

Embodiment 28. The method of embodiment 27, wherein the antibody binds to the cell surface receptor CS3CR1.

Embodiment 28. The method of embodiment 23, wherein the chelator is selected from EDTA and EGTA.

Embodiment 30. The method of any of the foregoing embodiments, wherein the allograft comprises an organ or part of an organ selected from the group consisting of lung, kidney, pancreas, intestine, and liver.

Embodiment 31. A preservation solution for a harvested allograft, the solution comprising: (i) an agent inhibits the activity of non-classical monocytes; and (ii) a chelator of divalent cations.

Embodiment 32. A composition comprising: (a) a harvested allograft; and (b) at least one of (i) an agent inhibits the activity of non-classical monocytes; and (ii) a chelator of divalent cations.

### EXAMPLES

The following Examples are illustrative and are not intended to limit the scope of the claimed subject matter.

### Example 1

Reference is made to the invention disclosure entitled “Pulmonary Intravascular Non-Classical Monocytes Mediate Lung Transplant Ischemia-reperfusion Injury,” Ankit Bharat and G. R. Scott Budinger, submitted Sep. 19, 2016, which contents are discussed below.

Technical Field

We have discovered that endothelial bound intravascular non-classical monocytes are retained in the donor lungs despite flushing them with the currently available preservative solutions. Upon implantation, these non-classical monocytes are activated and recruit neutrophils leading to primary graft dysfunction. Depletion or inhibition of non-classical monocytes in the donors is sufficient to ameliorate primary graft dysfunction. Since primary graft injury is the predominant cause for short-term mortality and chronic allograft rejection, our proposed strategy to deplete non-classical monocytes in the donor has the potential to significantly improve the outcomes following solid organ transplantation. We propose the development of novel pharmacological agents that can be administered directly to the donors at the time of procurement or be added to the preservative solutions to deplete the non-classical monocytes. Further, since non-classical monocytes are likely present in other organs, we propose that the strategy to deplete non-classical monocytes would be beneficial in other solid organ transplantation including kidney, liver, hearts, pancreas, and intestines.

Abstract

Primary graft dysfunction (PGD) is the predominant risk factor for both short-term and long-term allograft failure. Despite advances in the immunosuppressive regimens and preservative solutions, PGD has not been ameliorated. The cycle of ischemia-reperfusion that occurs during the procurement, transport, and re-implantation of the organ is speculated to cause the recruitment and activation of neutrophils into the transplanted allograft, which then initiates the inflammatory cascade and mediate primary graft dysfunction. While depletion of neutrophils in the recipient can possibly abrogate PGD, this strategy will also suppress the ability of the host to mount immunity against pathogens.

We have identified that a subset of non-classical monocytes is retained in the donor lungs and are responsible for recruitment of neutrophils after implantation. Depletion of these monocytes in the donors was found to prevent primary graft dysfunction. Therefore, we propose to develop pharmacological agents to deplete or inhibit these monocytes in human donors at the time of organ procurement by adding specific therapeutic agents to the currently used preservative solutions, developing new preservative solutions, or administering it directly to the donor at the time of procurement. Further, this strategy can be used for all solid organ transplantation.

Applications

The applications of the disclosed technology include solid organ preservation for clinical transplantation including clinical transplantation of lung, heart, kidney, pancreas, intestine, and liver. As such, the applications of the disclosed technology can include limiting organ damage of transplanted organs during transplantation and improving the success rate of organ transplantation.

Advantages

The inventors have shown that it is advantageous to deplete or inhibit intravascular non-classical monocytes in a donor in order to abrogate recruitment of neutrophils to the transplanted organs immediately following transplantation and prevent of neutrophil mediated injury to an allograft. As such, the disclosed technology is advantageous for limiting organ damage of transplanted organs during transplantation and improving the success rate of organ transplantation.

Brief Summary of Technology

The inventors' data show that a single injection of a chemical, clodronate, loaded on liposomes can induce apoptosis of non-classical monocytes and selectively deplete them in donor organs. Therefore, the inventors propose using the same strategy for clinical transplantation. Any chemical that induces cell death and is ingested by these non-classical monocytes which demonstrate phagocytic properties can be used for these purposes. Non-classical monocytes also have a cell surface protein SLAN and cytotoxic antibodies against this protein can be used to deplete non-classical monocytes in the methods disclosed herein. The inventors' data also show that inhibition of the cell surface receptor CX3CR1 can suppress the function of non-classical monocytes. Hence, agents that inhibit and/or deplete non-classical monocytes when injected into allograft donors intravenously or used in a preservative solution can be used to inhibit and/or deplete non-classical monocytes prior to transplantation of an allograft. Finally, because non-classical monocytes are bound to the endothelium of allografts via covalent bonds which are dependent on the presence of calcium and magnesium ions, ionic chelators, including but not limited to EDTA and EGTA, can be added to a preservative solutions or rinsing solution to facilitate dissociation of non-classical monocytes from allografts at the time of procurement.

### Example 2

Reference is made to Zheng et al. “Donor pulmonary intravascular nonclassical monocytes recruit receipient neutrophils and mediate primary lung allograft dysfunction,” Science Translational Medicine 14 Jun. 2017: Vol. 9, Issue 394, eea14508, the content of which is incorporated herein by reference in its entirety.

Abstract

Primary graft dysfunction is the predominant driver of mortality and graft loss following lung transplantation. Recruitment of neutrophils as a result of ischemia reperfusion injury is thought to cause primary graft dysfunction; however, the mechanisms that regulate neutrophil influx into the injured lung are incompletely understood. We found that donor-derived intravascular non-classical monocytes (NCM) are retained in human and murine donor lungs used in transplantation and can be visualized at sites of endothelial injury following reperfusion. When NCM in the donor lungs were depleted, either pharmacologically or genetically, neutrophil influx and lung graft injury was attenuated in both allogeneic as well as syngeneic models. Similar protection was observed when the patrolling function of donor NCM was impaired by deletion of fractalkine receptor CX3CR1. Unbiased transcriptomic profiling revealed upregulation of MyD88-pathway genes and a key neutrophil chemoattractant, CXCL2, in donor-derived NCM following reperfusion. Reconstitution of NCM-depleted donor lungs with wild type but not MyD88-deficient NCM rescued neutrophil migration. Donor NCM, through MyD88 signaling, were responsible for CXCL2 production in the allograft and neutralization of CXCL2 attenuated neutrophil influx. These findings suggest that therapies to deplete or inhibit NCM in donor lung might ameliorate primary graft dysfunction with minimal toxicity to the recipient. In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Introduction

Primary graft dysfunction (PGD), which develops in over 50% of lung allograft recipients, is the strongest risk factor for short-term mortality, early graft loss, and chronic rejection (1-4). Recipient neutrophils are the primary effector cells that are recruited to the allograft and lead to the development of PGD (5-11). Following reperfusion, neutrophils are recruited from the circulation and extravasate into the alveolar space of the lung allograft where they undergo NETosis, resulting in tissue damage and further activating the inflammatory cascade (6, 12). Strategies to abrogate neutrophil influx and activation are expected to reduce PGD and improve short- and long-term outcomes in patients undergoing lung transplantation. This could be accomplished by systemic depletion of neutrophils prior to lung transplantation, however, this strategy is not clinically practical given the importance of neutrophils in host defense and pathogen clearance (7-11).

In both humans and mice, there are at least two distinct populations of peripheral blood monocytes, classical and non-classical, which can be distinguished based on their surface marker expression and behavior (13, 14). Classical monocytes are known to leave the circulation in response to injury. Upon extravasation, they differentiate into inflammatory macrophages, playing important roles in the innate immune response to injury. Non-classical monocytes (NCM) adhere to the microvasculature, patrolling the endovascular space. They have been shown to inhibit the metastatic spread of tumors and to promote the inflammatory response to viral infection, nephritis, and inflammatory arthritis via mechanisms that are incompletely understood (15-21). In previous studies of lung transplantation and ischemia-reperfusion, nonselective strategies to deplete monocytes reduced the severity of neutrophil egress into the lung (11). Because donor lungs are vigorously perfused to remove any intravascular cells prior to transplantation, these studies have focused on studying recipient-derived classical monocytes and their role in the development of PGD (11). While donor-derived lung-resident cells such as alveolar macrophages have also been studied in the context of PGD, it was not until recently that our group and others reported the presence of donor-derived monocytes within lungs used in transplant (22, 23). As a result, detailed descriptions of these donor-derived monocytes and whether they play a role in the development of PGD has not yet been studied.

Here we report that NCM are present in murine donor lungs and are retained in the intravascular space despite vascular flushing. In murine models of syngeneic and clinically relevant allogeneic transplantation, pharmacologic or genetic depletion of NCM in the donor lungs prior to transplantation dramatically reduced the influx of neutrophils into the alveolar space and improved physiologic markers of lung allograft injury. Adoptive transfer of wild type NCM into mice deficient in NCM restored neutrophil influx and lung injury after transplantation. Signaling through the adaptor protein MyD88 in NCM was required to generate macrophage inflammatory protein-2 (CXCL2 or MIP-2), which was necessary for post-transplant neutrophil infiltration of the lung allograft. We confirmed that NCM are retained in human donor lungs used in clinical transplantation, and that the post-perfusion allograft experiences a brisk neutrophil influx, similar to our murine model. These findings suggest that therapies that deplete or inhibit the function of NCM might ameliorate PGD without impairing the host response to infection. Because these therapies can be selectively targeted to the donor lung, the toxicity to the recipient is predicted to be minimal.

Results

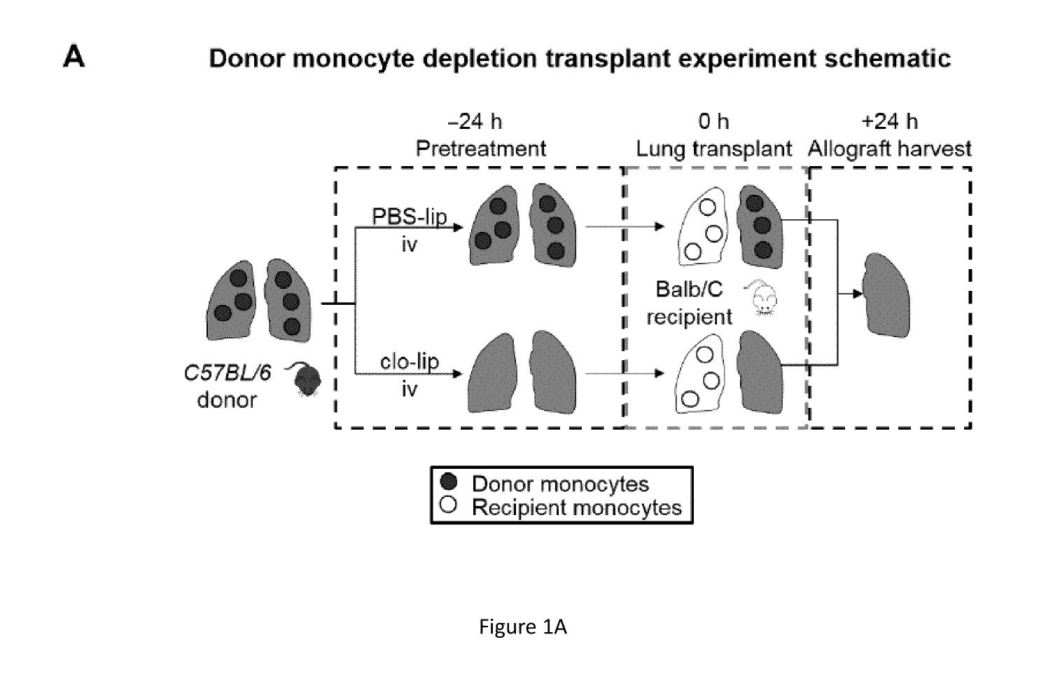

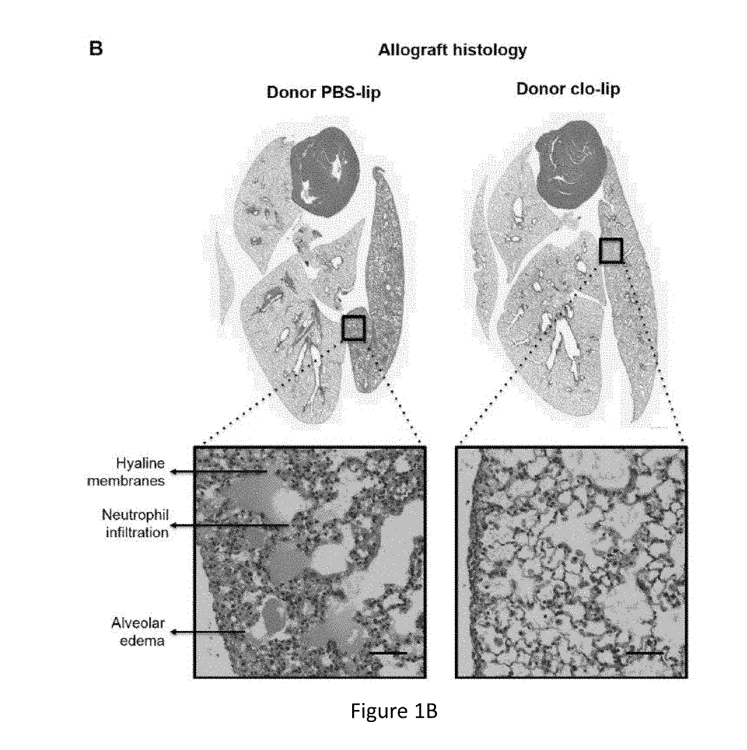

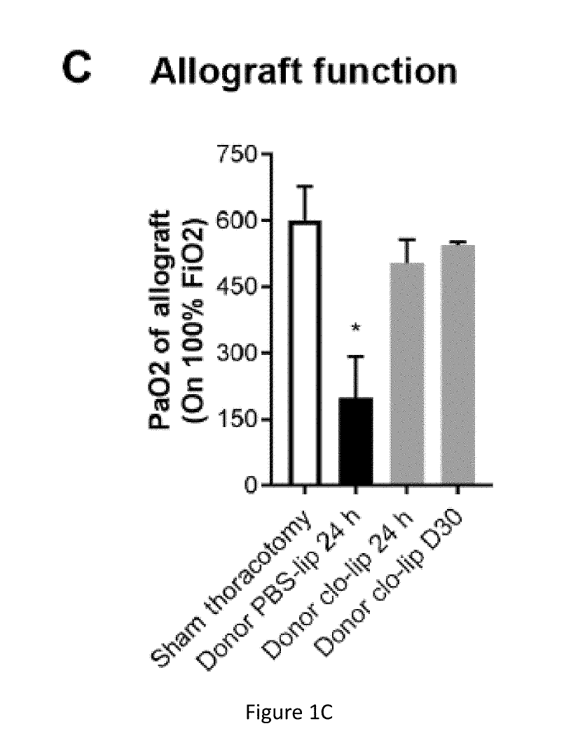

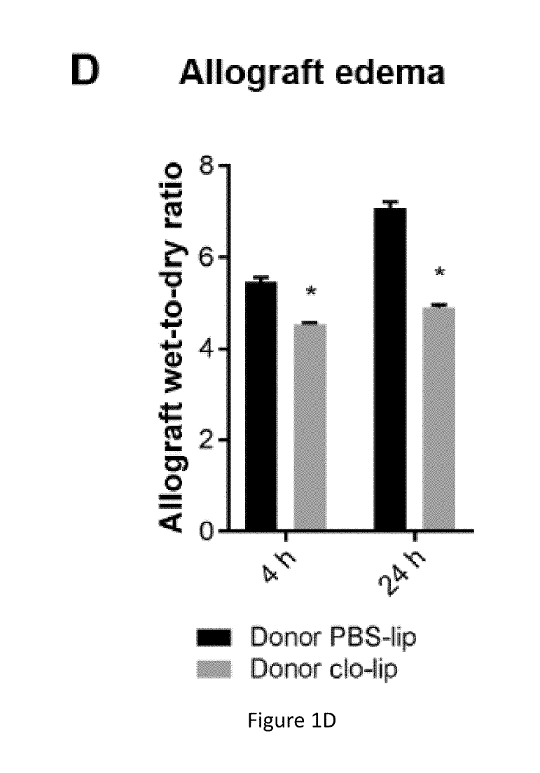

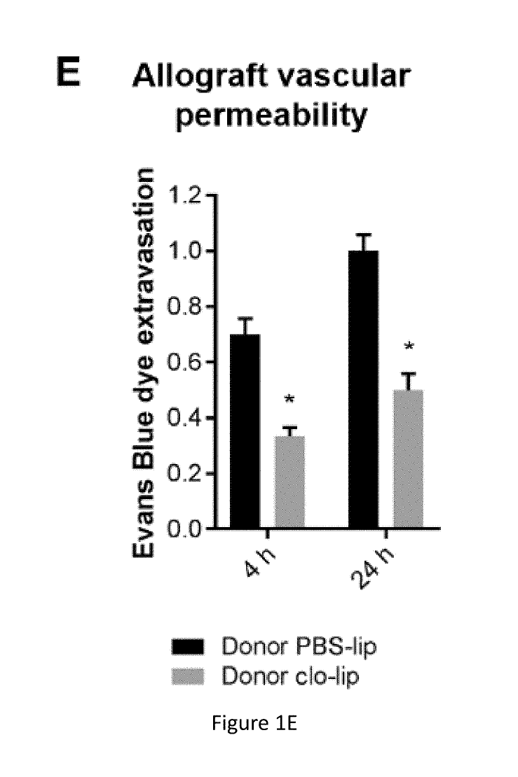

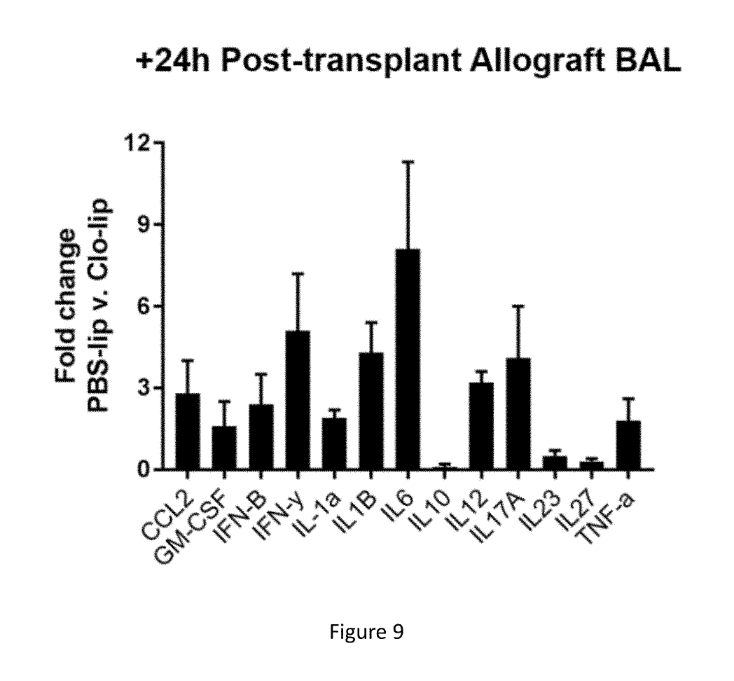

Donor monocyte depletion confers protection against primary graft dysfunction following murine lung transplant. Given our previous unexpected finding of retained monocytes within the donor lung (22), we sought to test the hypothesis that depletion of donor-derived monocytes would ameliorate PGD using a reproducible and clinically relevant murine model of allogeneic lung transplantation. Since clodronate-loaded liposomes (clo-lip) efficiently deplete all monocyte subtypes in circulation (24, 25), we treated murine donors with clo-lip 24 hours prior to transplantation and measured the severity of PGD following transplantation (FIG. 1A). As shown in FIG. 1B, when donors were treated with clo-lip, gross and microscopic examination of the lung allograft showed no signs of lung injury and revealed preserved architecture without alveolar edema or hyaline membrane formation. In contrast, donors that received phosphate buffered saline-loaded liposomes (PBS-lip) had signs of severe allograft injury and PGD development, including alveolar edema, capillaritis, and hyaline membrane development. Allografts from donors treated with clo-lip revealed demonstrated normal oxygenation, while PBS-lip-treated mice had significantly impaired gas exchange (FIG. 1C). Native recipient lungs in both groups were preserved and hearts revealed no damage. Donor clo-lip treatment protected against post-reperfusion pulmonary edema as indicated by multiple measures including decreased wet-dry ratio (FIG. 1D) and capillary leak determined using Evans blue dye extravasation (FIG. 1E). Additionally, pro-inflammatory cytokines in bronchoalveolar lavage fluid (BALF) were suppressed in clo-lip treated donors compared to PBS-lip (FIG. 9). Finally, to investigate whether clo-lip treatment had any detrimental long-term effects on the allograft function, we transplanted wild type Balb/c lungs following clo-lip treatment of donors into wild type C57B1/6 recipients and analyzed allograft function on day 30. To avoid allograft rejection resulting from de novo alloimmunity, we administered co-stimulatory blockade using a combination of MHC-related 1 (MR1) and cytotoxic T-lymphocyte-associated protein 4-Ig (CTLA4-Ig), as previously described (27-29). All lung allografts revealed a PaO₂ of >500 mmHg indicative of normal function (mean 543±23). This indicated that donor monocyte depletion did not compromise long-term allograft function (FIG. 1C).

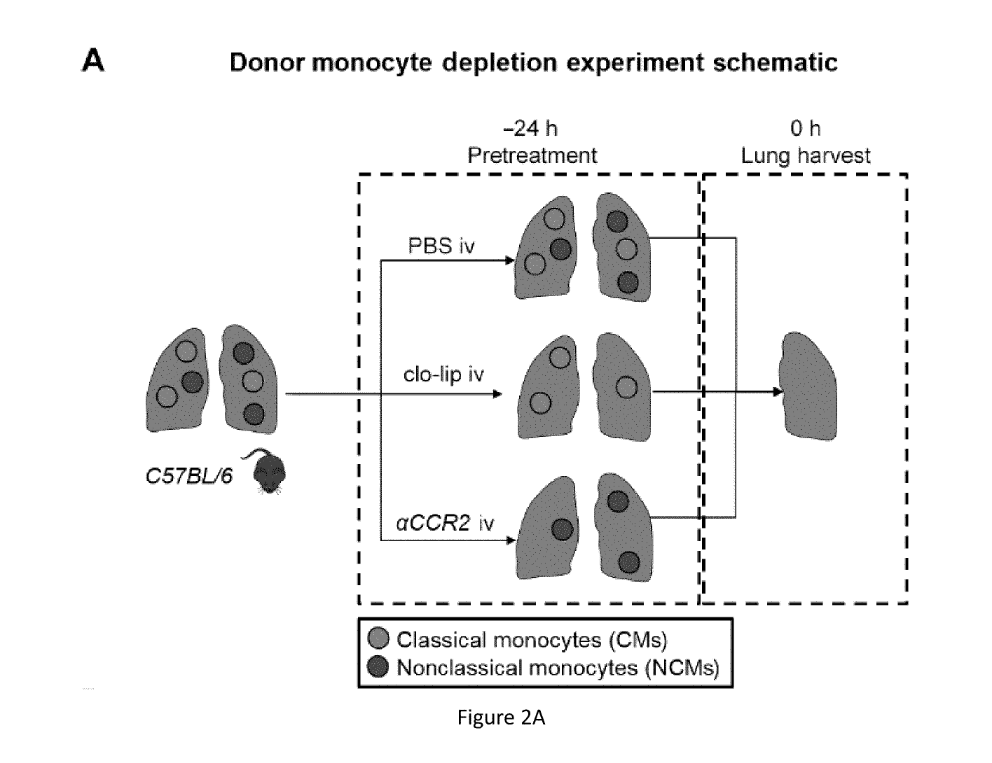

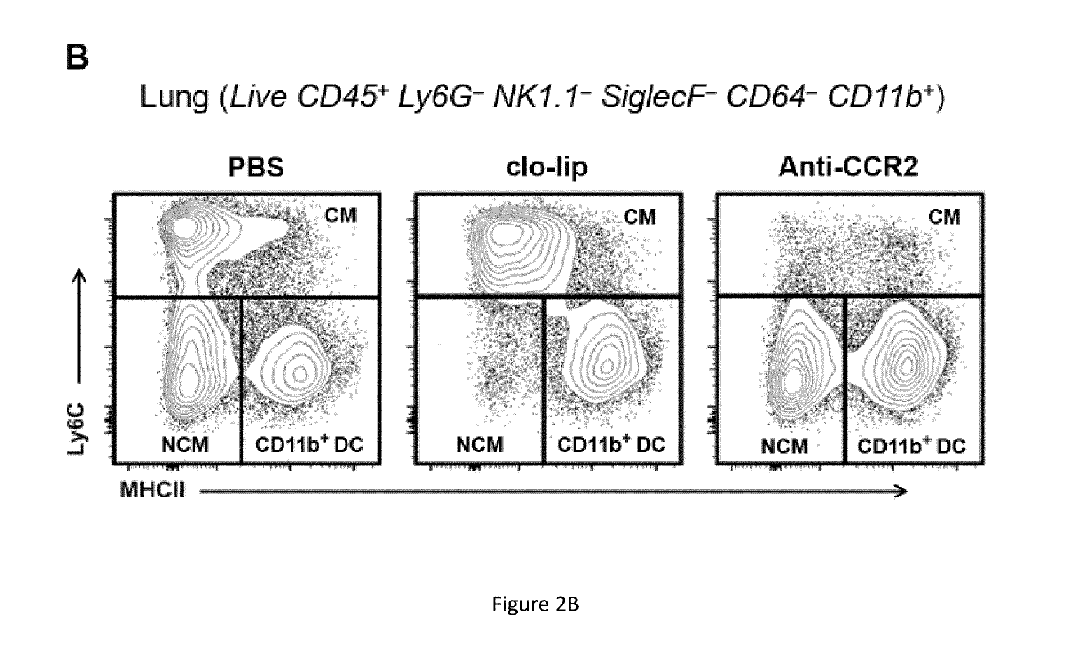

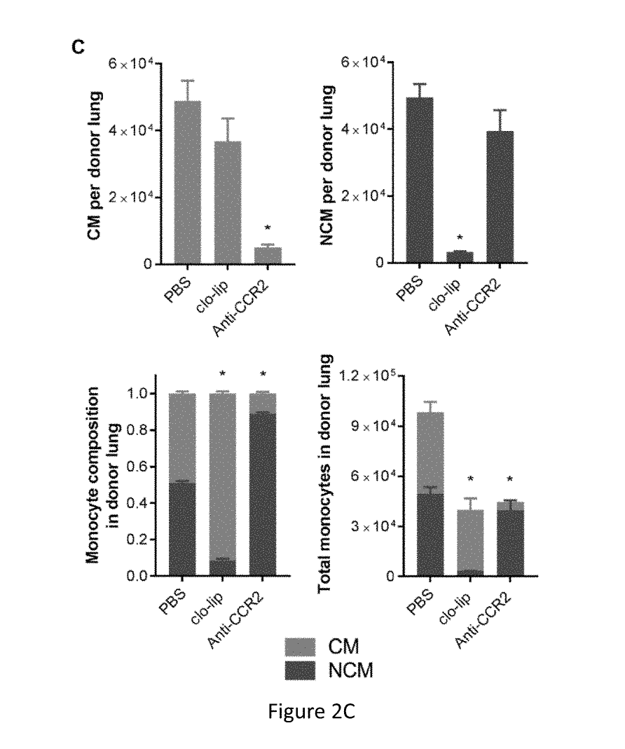

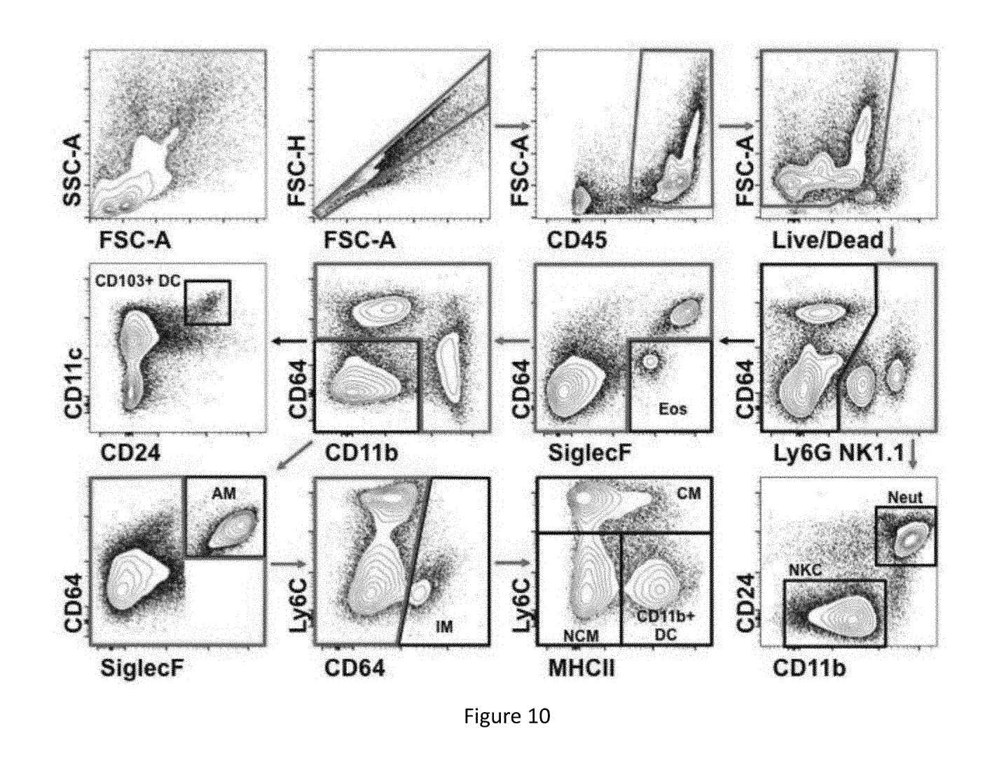

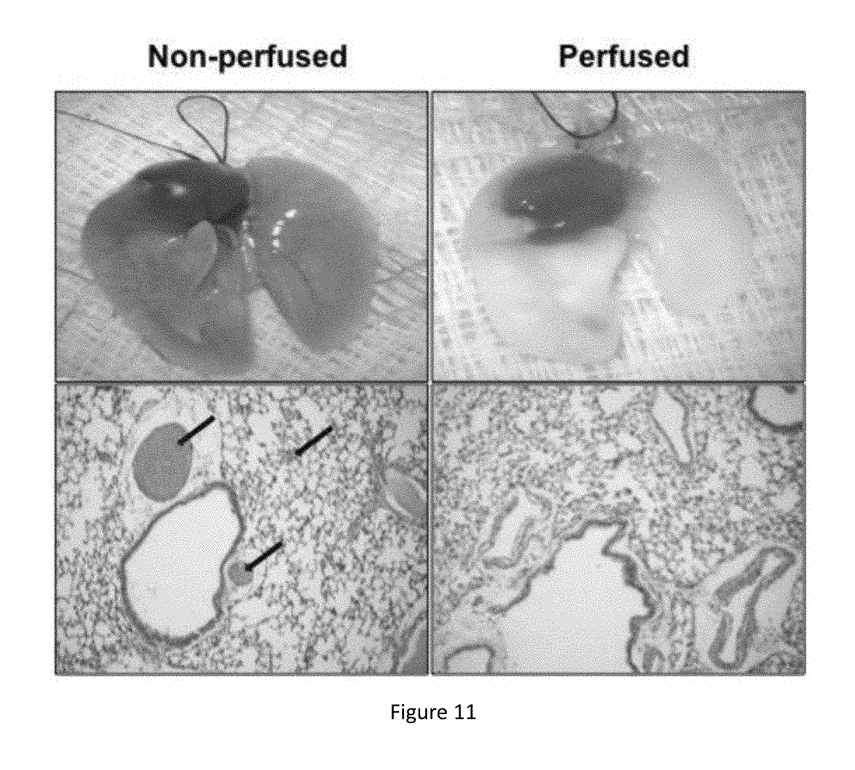

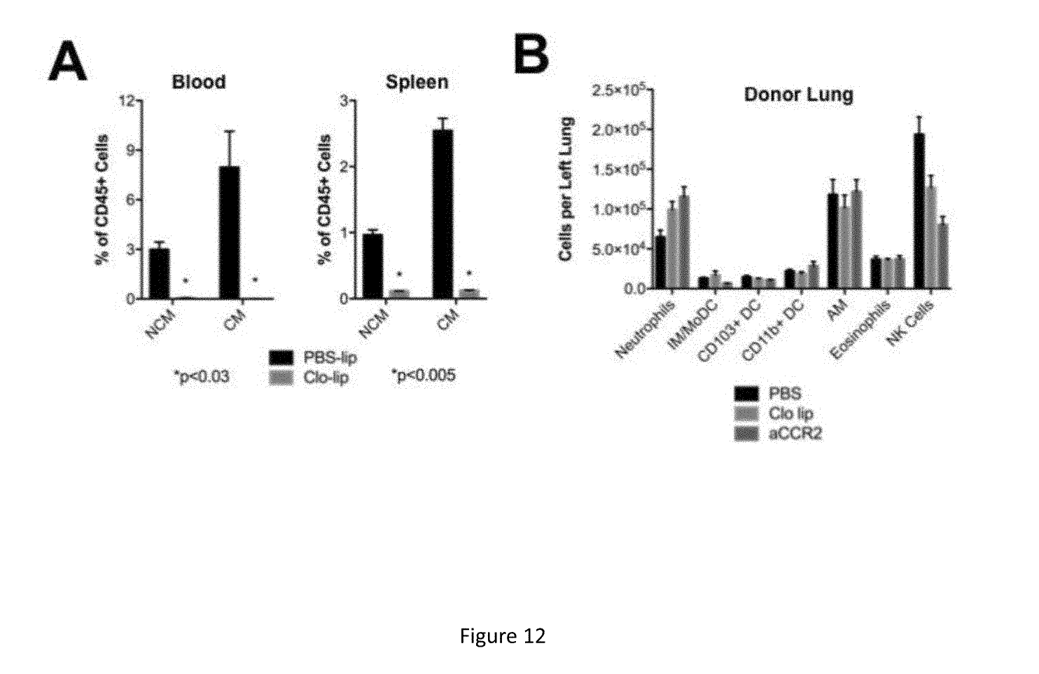

Intravenous clodronate-loaded liposomes selectively depletes donor pulmonary intravascular NCM in donor lungs. Having observed the marked amelioration of PGD, we sought to determine the specific pulmonary myeloid cell populations affected by intravenous clo-lip treatment of the donors (FIG. 2A). Using a previously established protocol (22), we identified the myeloid populations in the perfused murine lungs (FIG. 10). These murine lungs were perfused in a manner similar to in human lung donors(please see materials and methods for details). The perfusion of the lungs consistently eliminated circulating red blood cells (FIG. 11). Donor lungs were found to have CD45⁺Ly6G⁻NK1.1⁻SiglecF⁻CD64⁻CD11b⁺ monocytes, in addition to other myeloid cell populations. Of these monocytes, both non-classical Ly6C^{low} MHCII⁻ (3-5% of CD45⁺) and classical Ly6C^{high} MHCII^{+/-} (4-6% of CD45⁺) were identified. Non-classical monocytes (NCM) comprised ˜40-50% of total monocytes in flushed murine lungs. Intravenous injection of clo-lip completely depleted NCM, but not classical monocytes in the donor lungs (FIGS. 2B&C). This was in contrast to blood and spleen where both subpopulations of monocytes were depleted (FIG. 12A). Since liposomes administrated intravenously do not leave the intravascular compartment (17), intravenously administered clo-lip only eliminates intravascular, but not interstitial monocytes, indicating that non-classical and classical monocytes are compartmentalized in the lung intravascular and interstitial spaces, respectively. In contrast, intravenous injection of anti-CCR2 antibodies led to depletion of only classical monocytes but not NCM (FIGS. 2B&C) in the lungs. All other lung-resident myeloid cell populations were not affected by either clo-lip or anti-CCR2 treatment (FIG. 12B). This indicated that the protection by donor clo-lip treatment against PGD following transplantation was due to selective depletion of pulmonary intravascular NCM in the donor lungs.

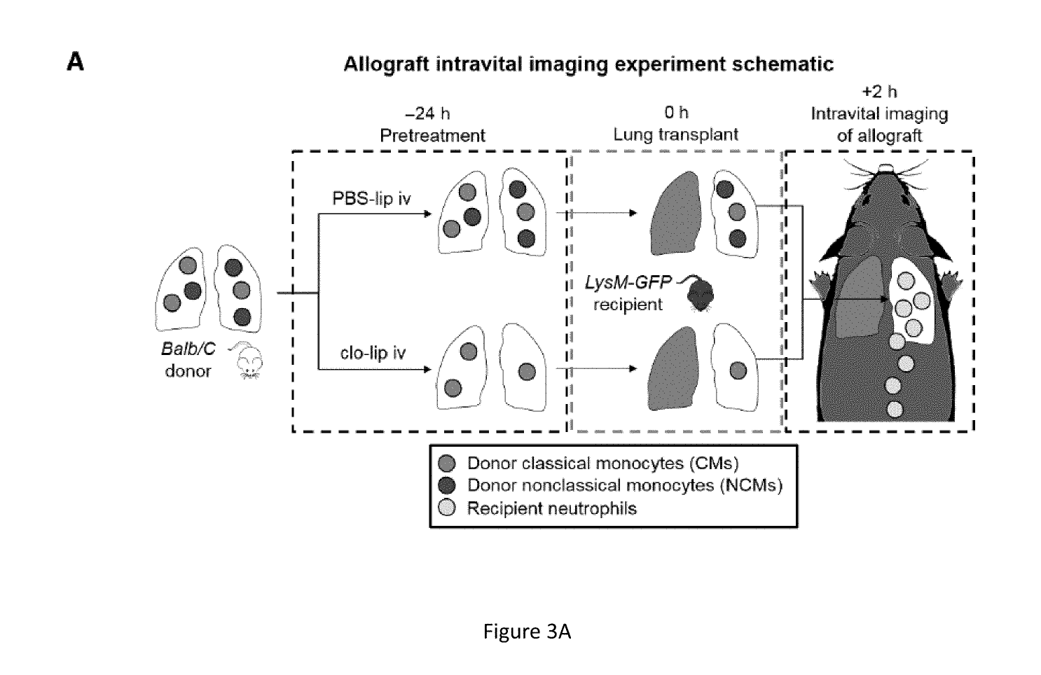

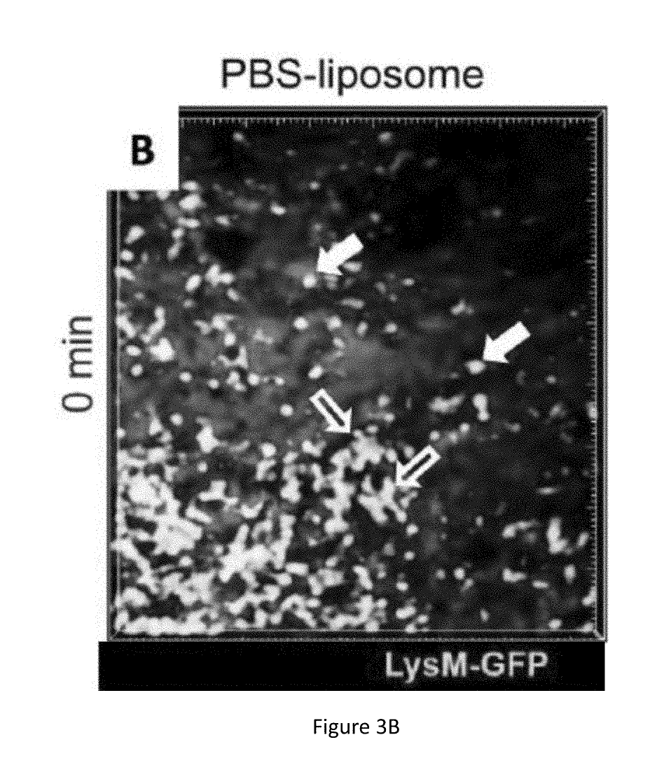

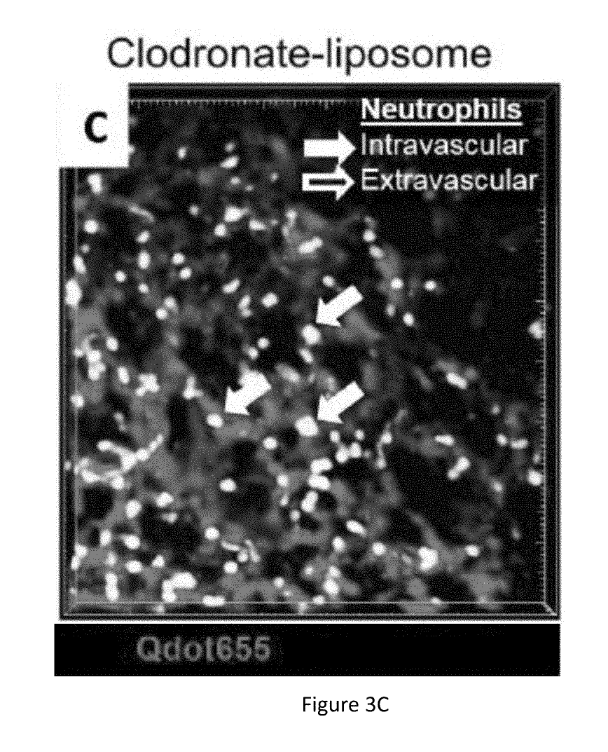

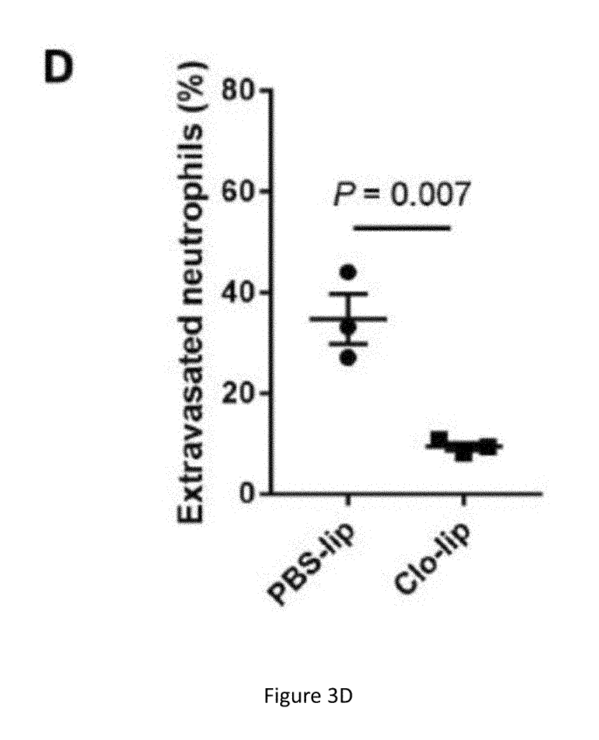

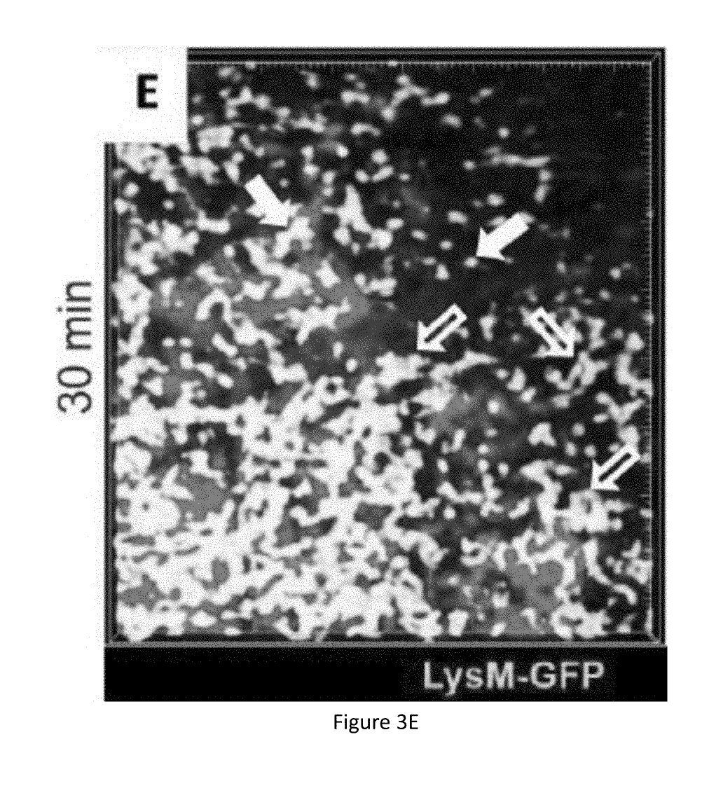

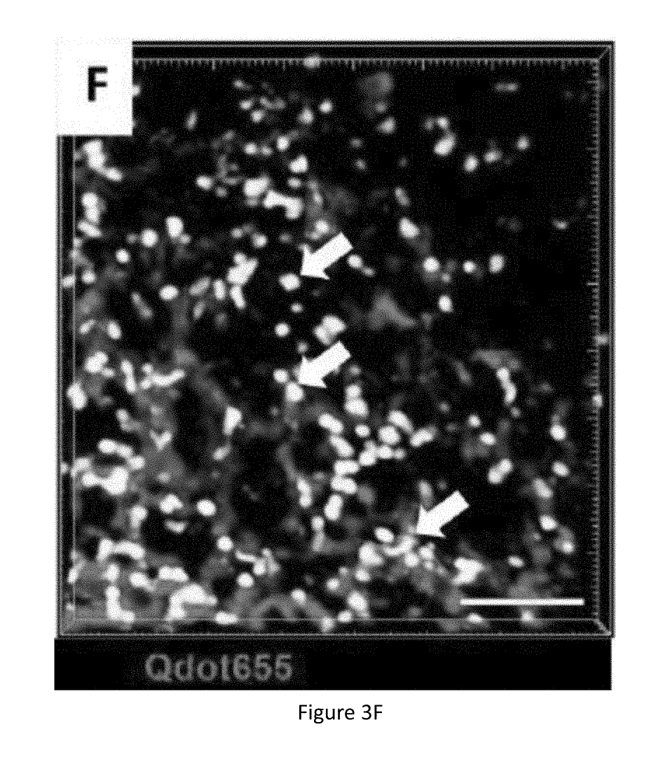

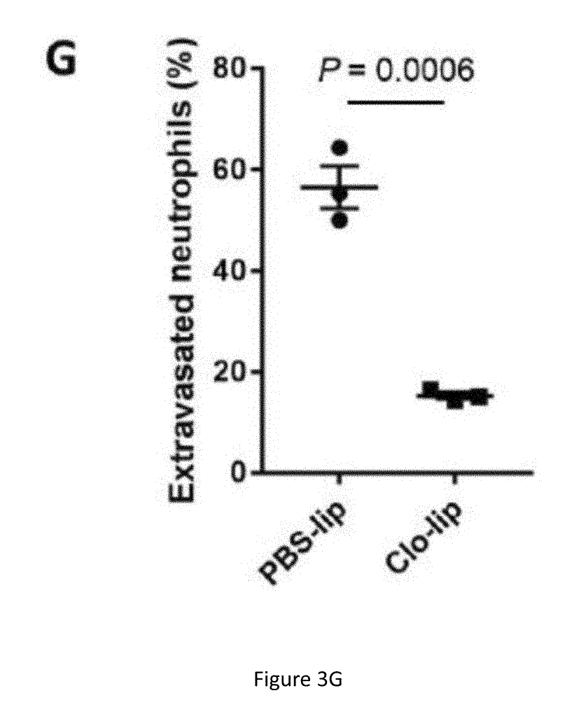

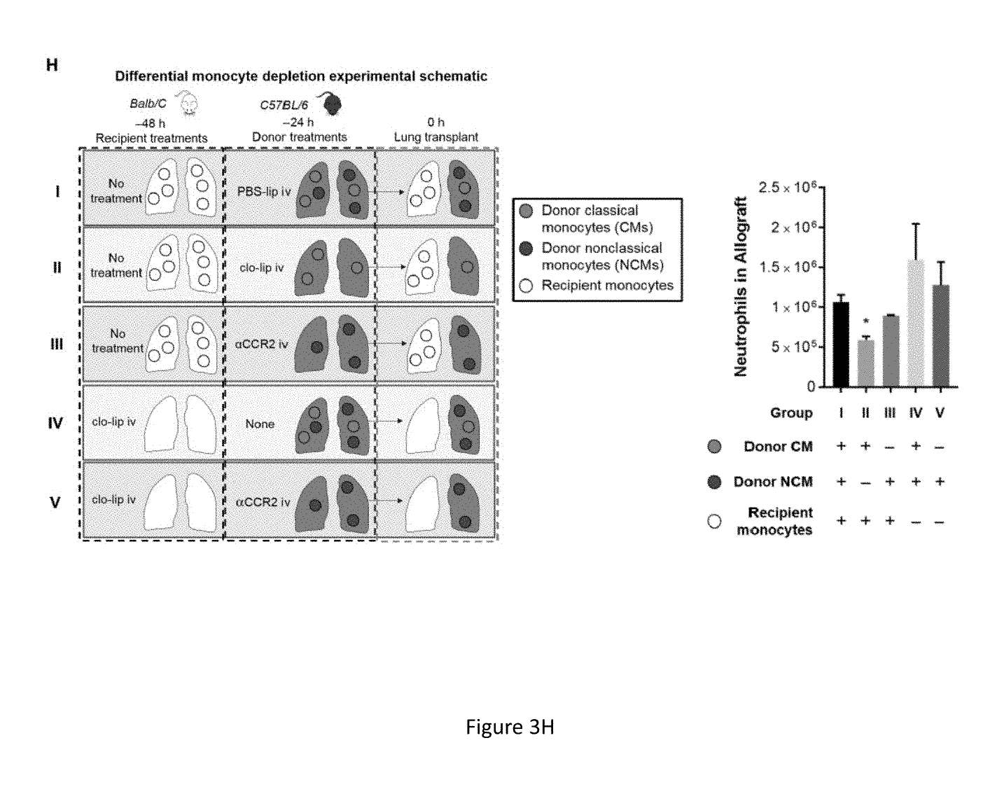

Depletion of donor intravascular NCM abrogates neutrophil influx following lung transplantation. Since PGD is mediated by neutrophils (26), we sought to determine whether depletion of donor pulmonary NCM abrogated neutrophil influx following reperfusion of the allograft. We used LysM-GFP mice as recipients, which express GFP in neutrophils, allowing us to examine the dynamic influx of recipient neutrophils into the lungs and monitor in real-time the rate of extravasation of recruited neutrophils after transplantation using intravital two-photon microscopy (FIG. 3A). Donors were treated intravaneously with clo-lip or PBS-lip as control 24-hours prior to transplantation. 2 hours after reperfusion, lungs from PBS-lip treated control donors demonstrated significant neutrophil extravasation (34.7±4.94% of total neutrophils were extravascular). However, treatment of donors with clo-lip reduced neutrophil extravasation (9.43±0.78% of total neutrophils, p=0.0072, FIG. 3B-D). Thirty minutes later, the proportion of extravasated neutrophils in PBS-lip treated control lung allografts continued to increase but much less so in clo-lip treated donor lungs, which had proportionally fewer extravasated neutrophils than control allografts (56.5±4.17% vs 15.3±0.64% of total neutrophils, p=0.0006, FIG. 3E-G).

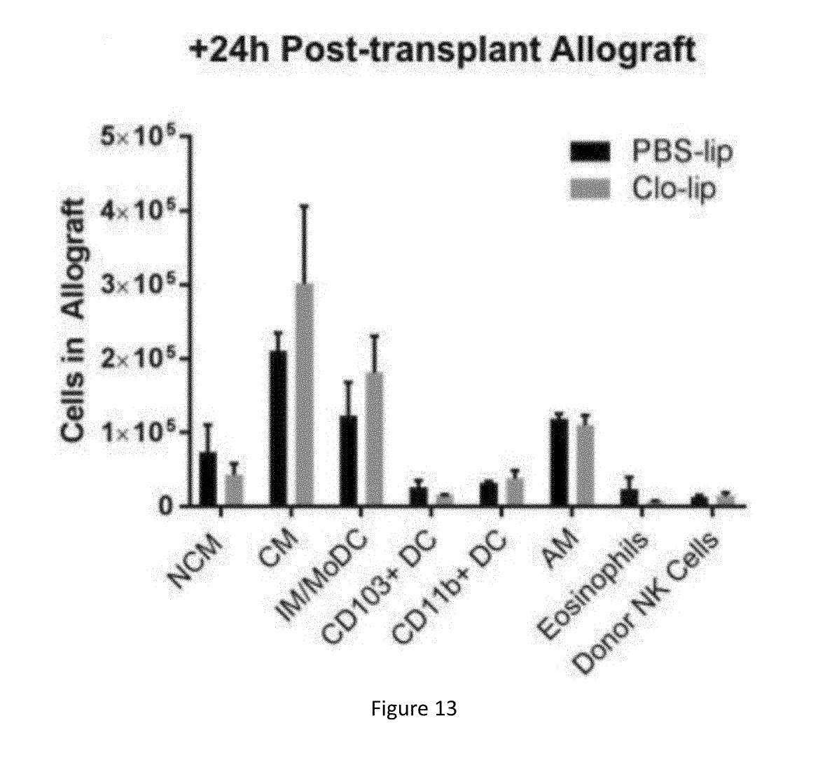

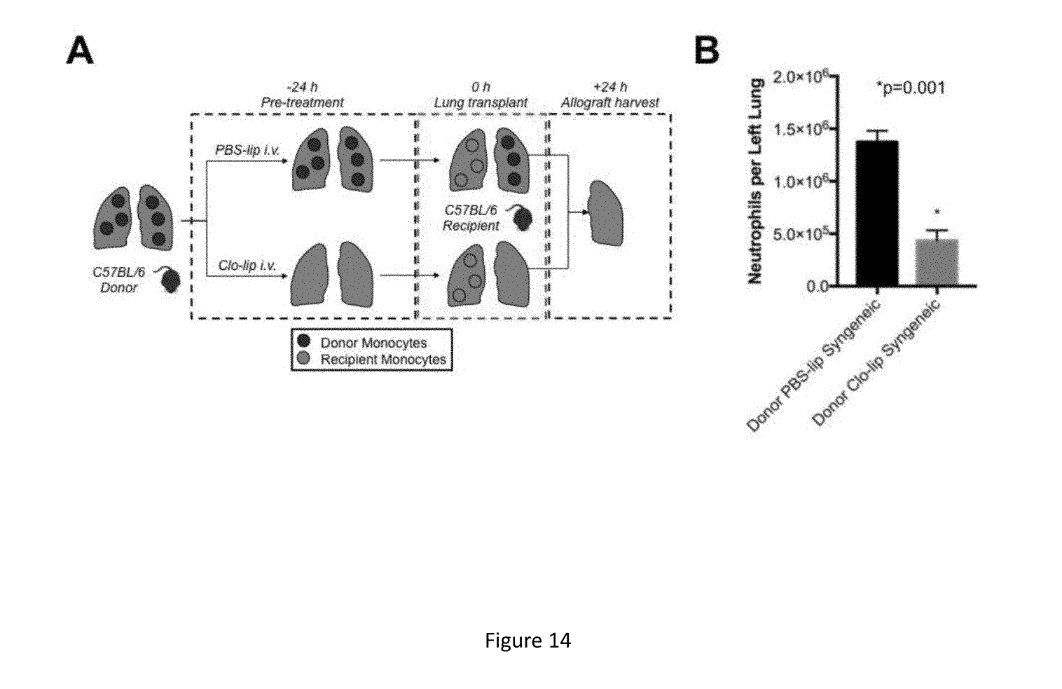

Since LysM-GFP mice can express GFP in other myeloid cells in addition to neutrophils, we used flow cytometry to confirm that pretreatment of donors before transplant with clo-lip leads to a significant suppression of neutrophil influx following allogeneic lung transplantation (FIG. 3H, Group 2). While neutrophil influx was suppressed, we did not find any changes in the influx of other myeloid cells (FIG. 13). At such early time points, it is generally accepted that there is no role for an alloimmune response in mediating neutrophil influx in naïve recipients (30). To confirm that alloimmunity did not play a role in neutrophil influx, we performed the same experiment using the syngeneic murine lung transplant model and found similar results (FIG. 14). To eliminate the possibility that the neutrophil recruitment was mediated by donor classical monocytes, we depleted them using anti-CCR2 antibodies before transplantation and found that neutrophil influx was unaltered in the absence of donor classical monocytes (FIG. 3H, Group 3). Next, we eliminated the possibility of recipient-derived monocytes driving the neutrophil influx following lung allograft reperfusion by treating the recipients with clo-lip 48 hours prior to transplant, a procedure we have shown results in profound monocytopenia for 72 hours (17). Wild type donor lungs transplanted into clo-lip treated recipients were not protected from neutrophil infiltration post-transplantation (FIG. 3H, Group 4). Finally, we depleted donor classical monocytes using anti-CCR2 antibodies and depleted all intravascular recipient monocytes with clo-lip, 48-hours prior to transplantation. This treatment, which left only NCM in the donor lungs, resulted in enhanced neutrophil infiltration after transplantation (FIG. 3H, Group 5).

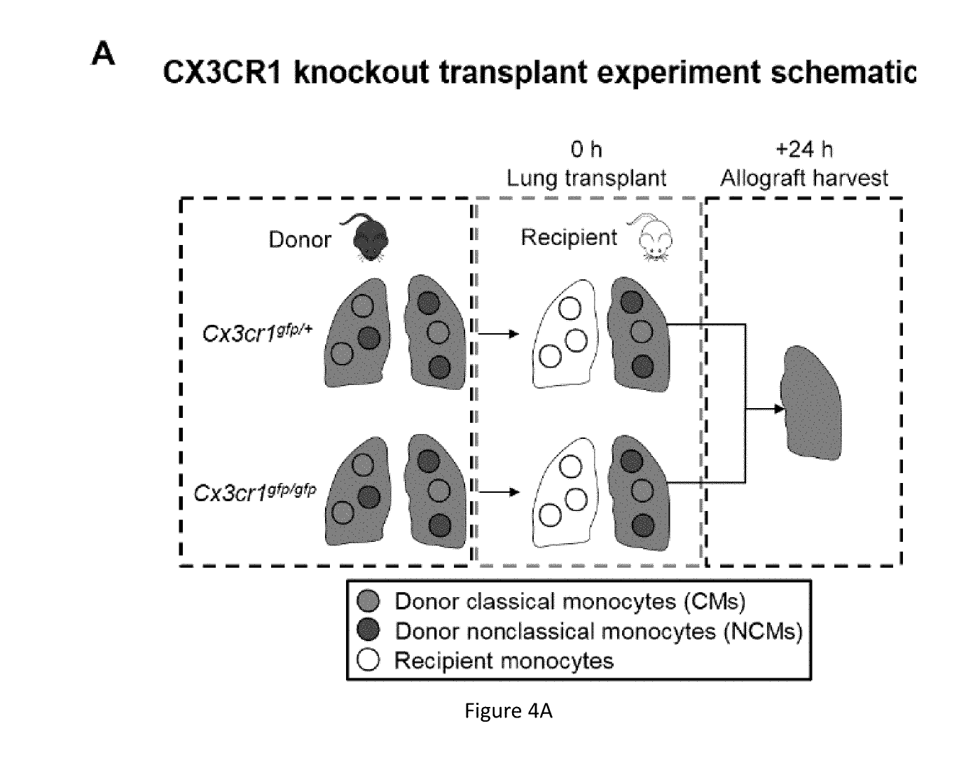

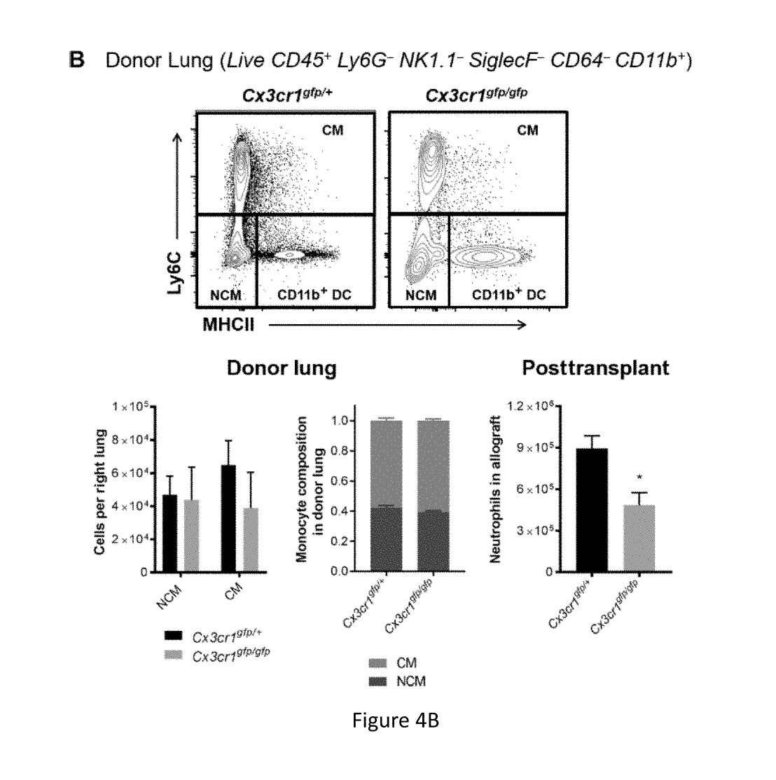

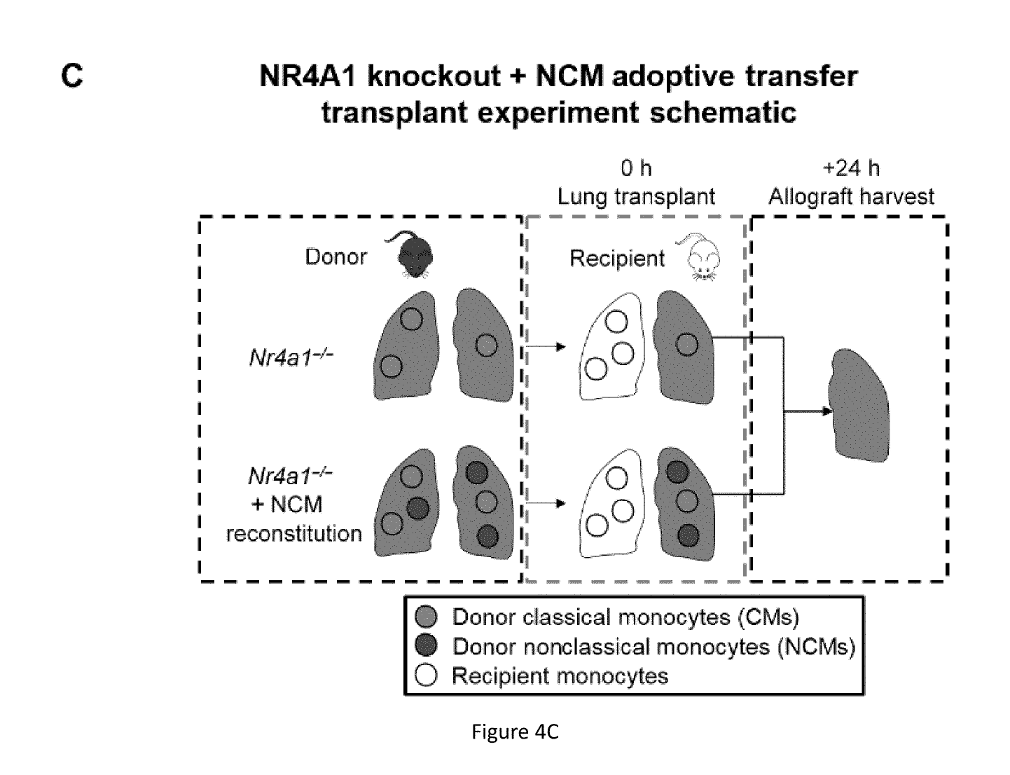

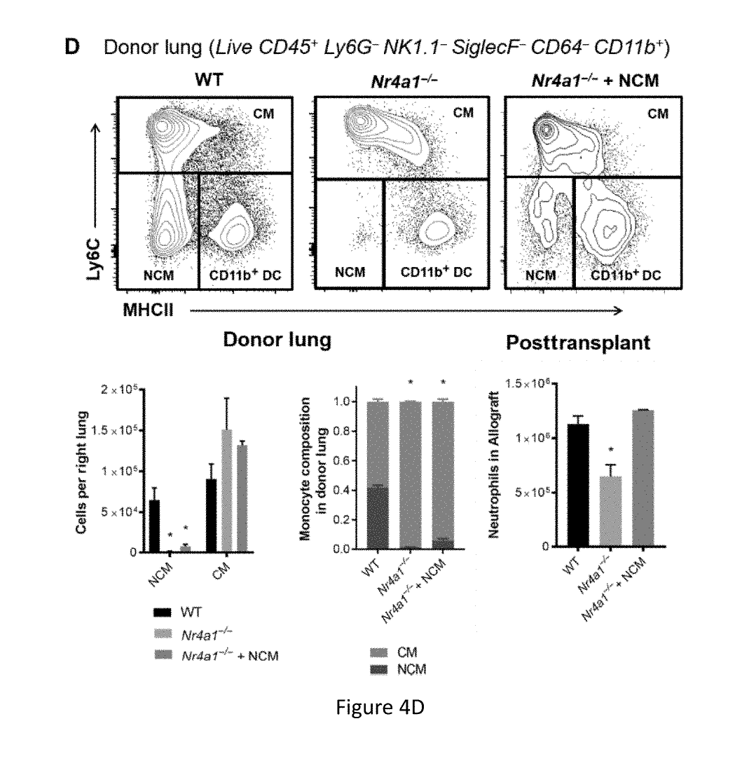

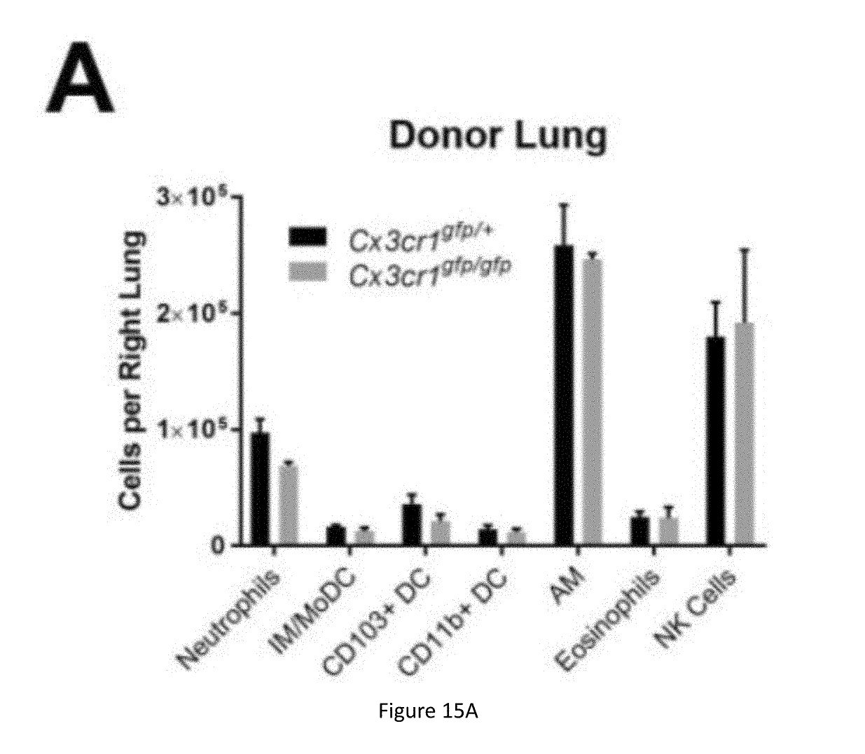

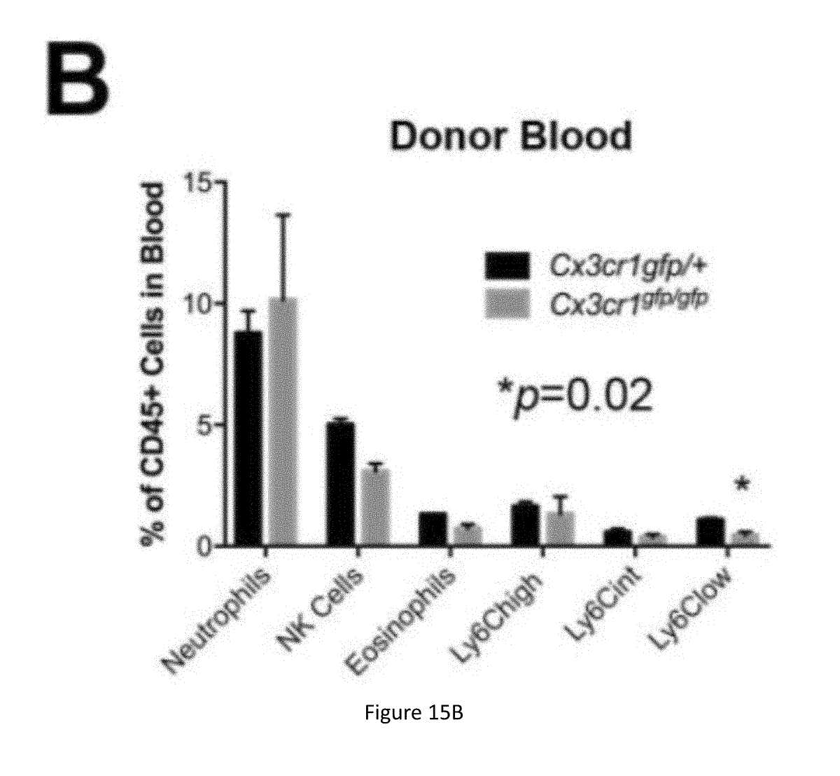

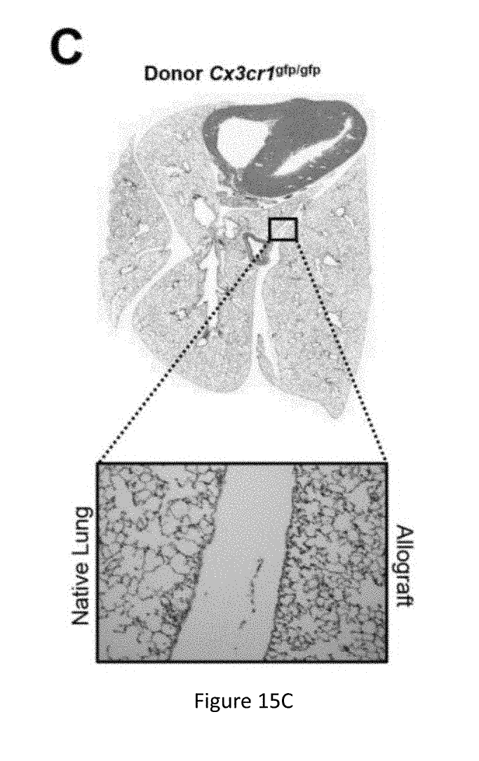

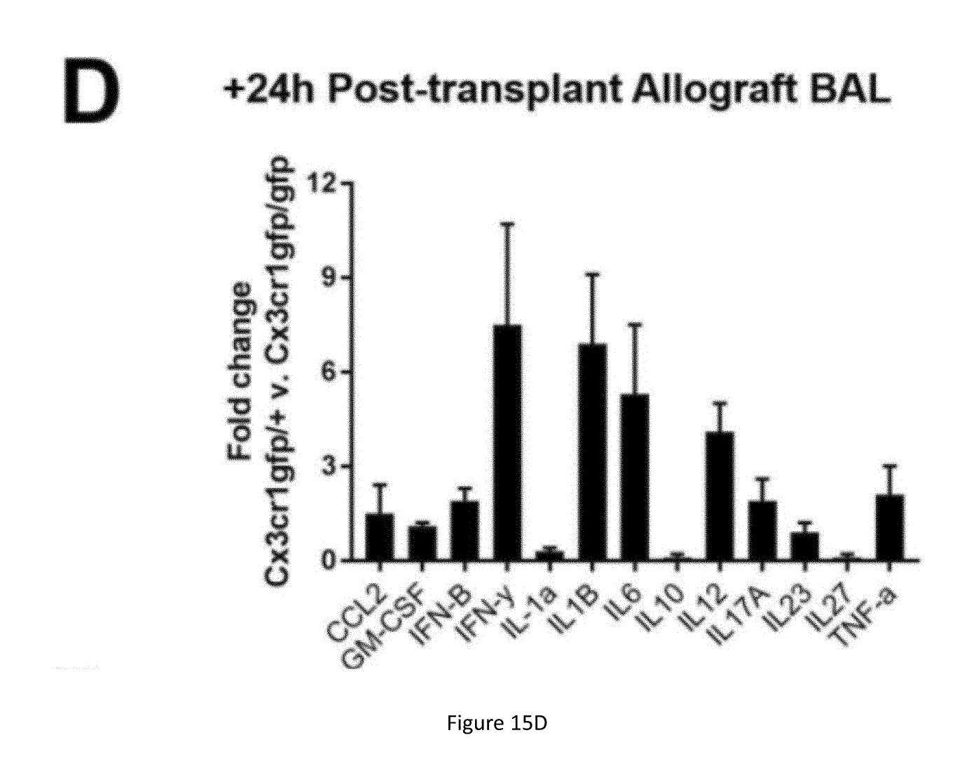

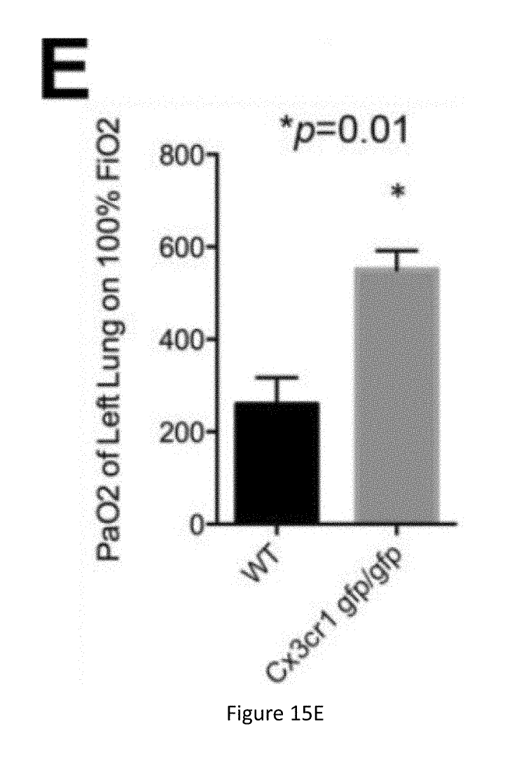

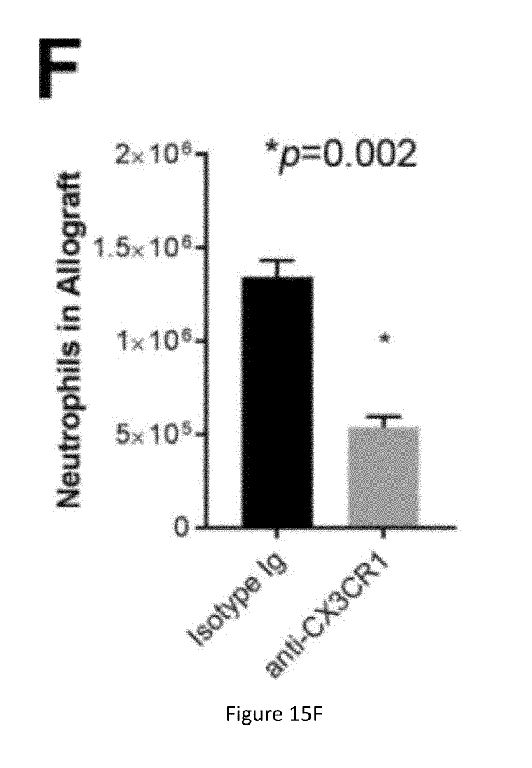

Donor NCM are necessary for neutrophil influx following reperfusion and development of PGD. We next confirmed the role of donor NCM in neutrophil trafficking into the lungs after transplant with two independent genetic models (FIG. 4A). The fractalkine receptor CX3CR1 is not necessary for the formation of NCM but is required for their patrolling function (19, 31). Consistently, we found that Cx3cr1^{gfp/gfp} mice, which have a homozygous knockin mutation of the fractalkine receptor, had preserved NCM and other myeloid cells in the lungs under homeostasis (FIG. 4B & FIG. 15). Heterozygous Cx3cr1^{gfp/+} mice had levels of NCM both in blood and lung similar to wild type mice (FIG. 4B & FIG. 15B). When Cx3cr1^{gfp/gfp} mice were used as lung donors, neutrophil influx was attenuated compared with Cx3cr1^{gfp/+} donor lungs (FIG. 4B). Cx3cr1^{gfp/gfp} donor lungs demonstrated preserved gas exchange and allograft architecture. Additionally, the levels of pro-inflammatory cytokines were reduced in the bronchoalveolar lavage at 24 hours when Cx3cr1^{gfp/gfp} donor lung were used (FIG. 15). We confirmed the role of CX3CR1 on donor NCM by using neutralizing anti-CS3CR1 IgG antibodies administered to the donor prior to the graft procurement and immediately following reperfusion. In contrast to isotype control antibodies, anti-CX3CR1 antibodies led to protection against neutrophil infiltration into the allograft similar to that observed in Cx3cr1^{gfp/gfp} donor lungs (FIG. 15).

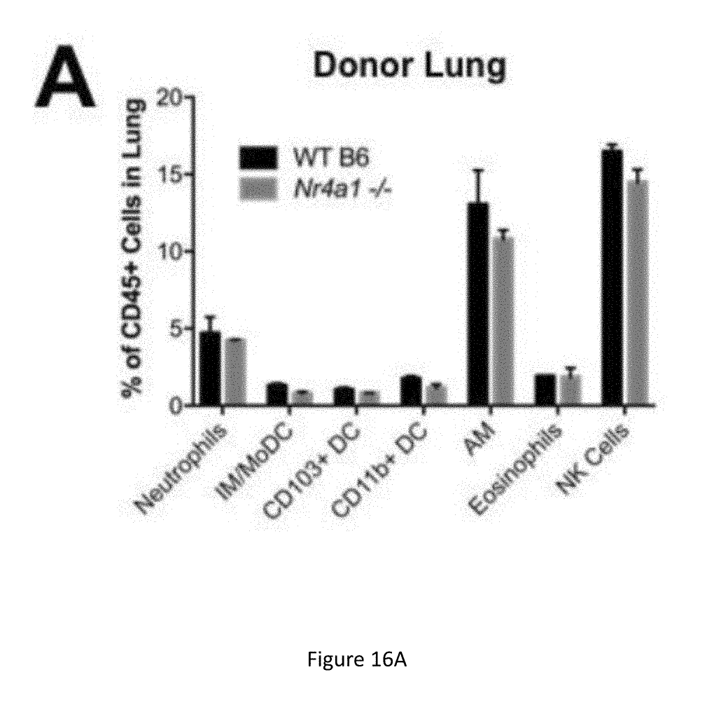

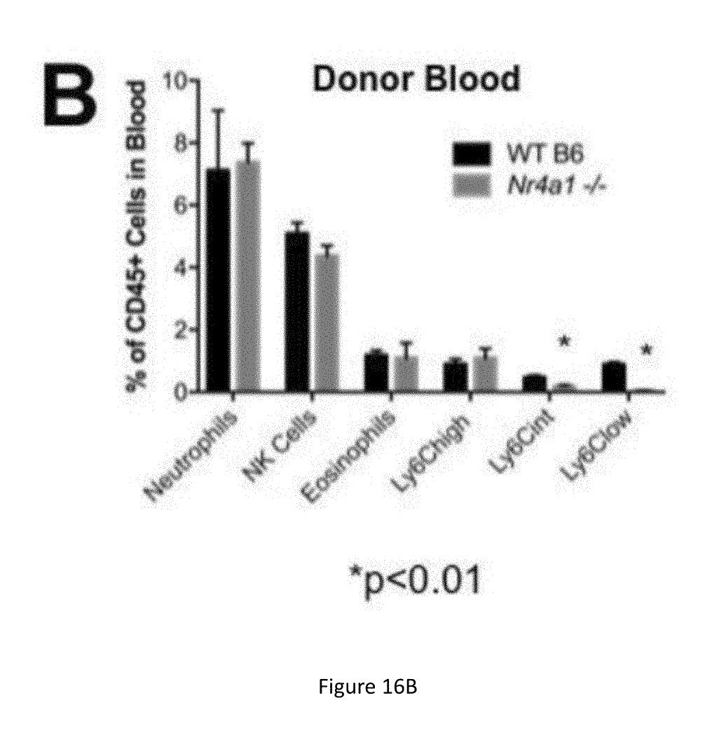

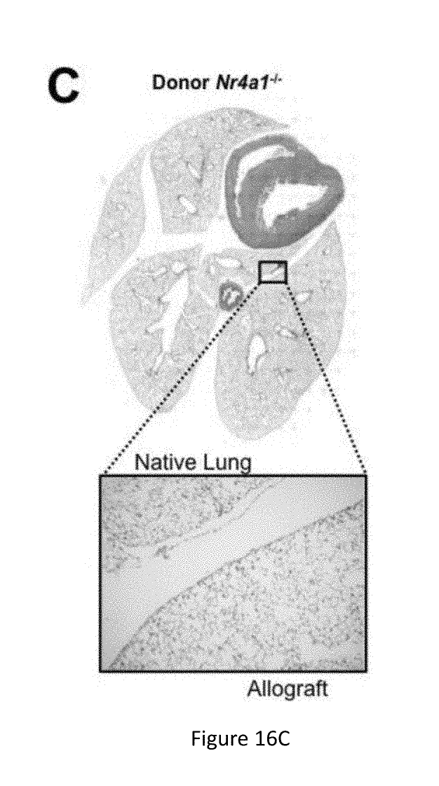

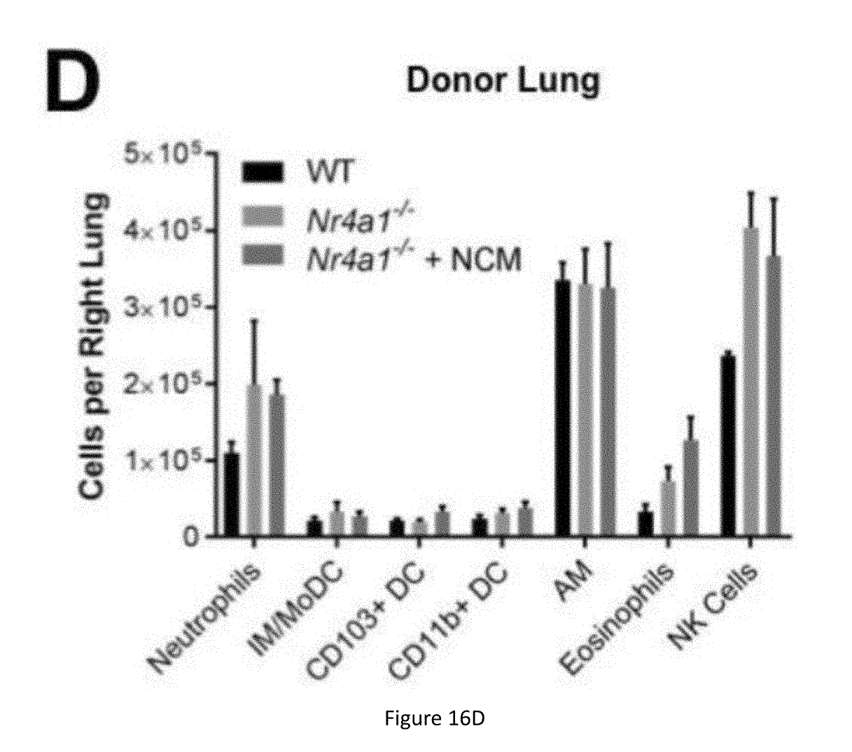

The orphan nuclear receptor NR4A1 (also known as Nur77) is required for the differentiation of classical monocytes into NCM, and although these Nr4a1^{-/-} mice develop normally, they lack NCM (32). Consistent with these findings, we did not detect NCM in perfused donor lungs from Nr4a1^{-/-} mice but other myeloid cell populations were preserved (FIG. 4D, FIG. 16A). Transplantation of Nr4a1^{-/-} donor lungs into wild type recipients was associated with reduced neutrophil recruitment and extravasation in the lung compared with allogeneic wild type donors into wild type recipients (FIG. 4D) and preserved allograft architecture (FIG. 16).

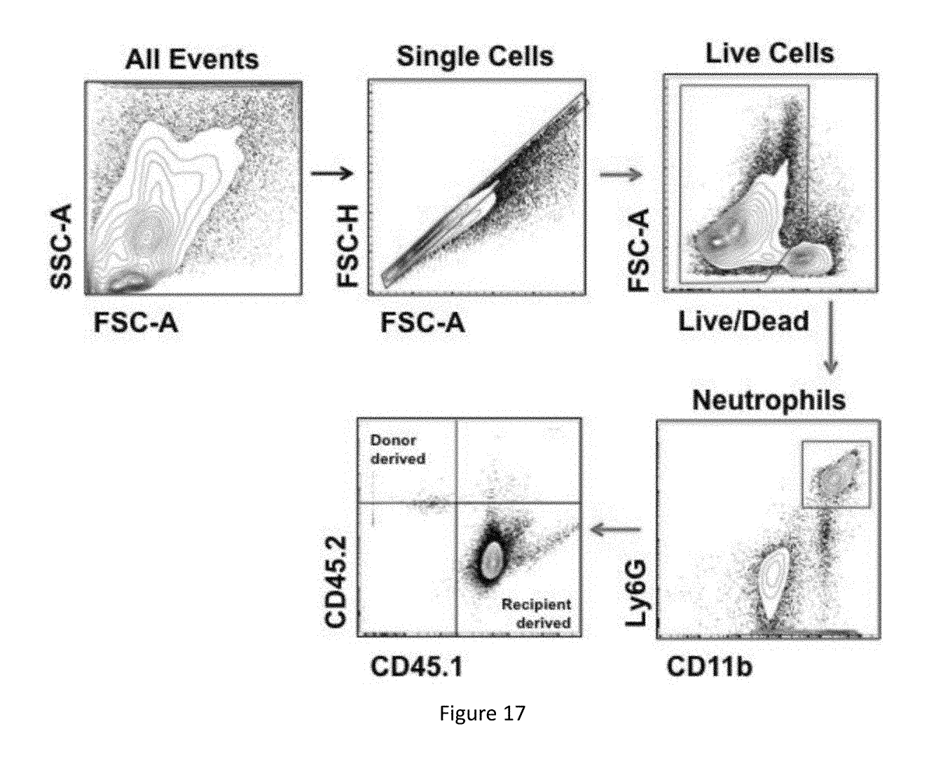

Given these findings, we reasoned that reconstitution of Nr4a1^{-/-} mice (B6 background) with isogenic Cx3cr1^{gfp/+} NCM (B6 background) should allow us to reverse the protection conferred by the loss of NR4A1. Use of functional Cx3cr1^{gfp/+} cells, which have the green-fluorescent protein on NCM, allowed us to confirm successful engraftment into the NR4A1 mice and track them following transplantation. Indeed, adoptive transfer of NCM restored the number of these cells in the NR4A1 murine lungs in the same spatial distribution (FIG. 4D). There was no difference in the other myeloid cell population in the reconstituted NR4A1 donor lungs (FIG. 16D). When these NR4A1-deficient lungs were reconstituted with Cx3cr1^{gfp/+} NCM and then used as donors, neutrophil influx following transplantation was restored (FIG. 4D). All neutrophils at 24 hours were of recipient origin (FIG. 17), consistent with the rapid recipient-derived neutrophil influx observed during two-photon imaging of LysM-GFP recipients.

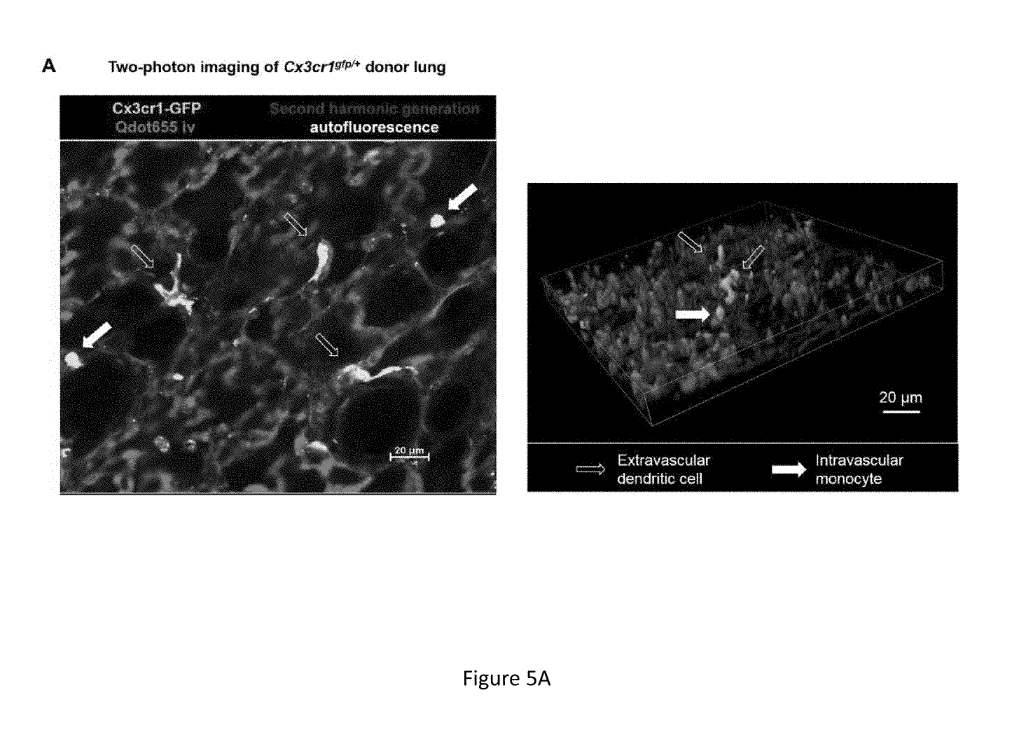

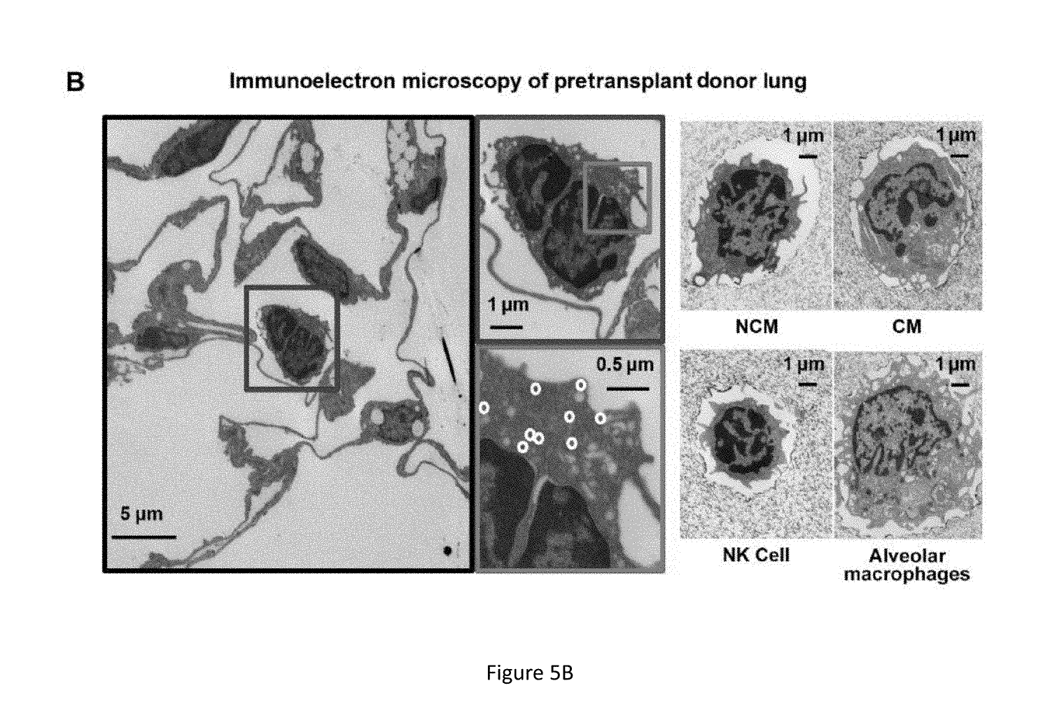

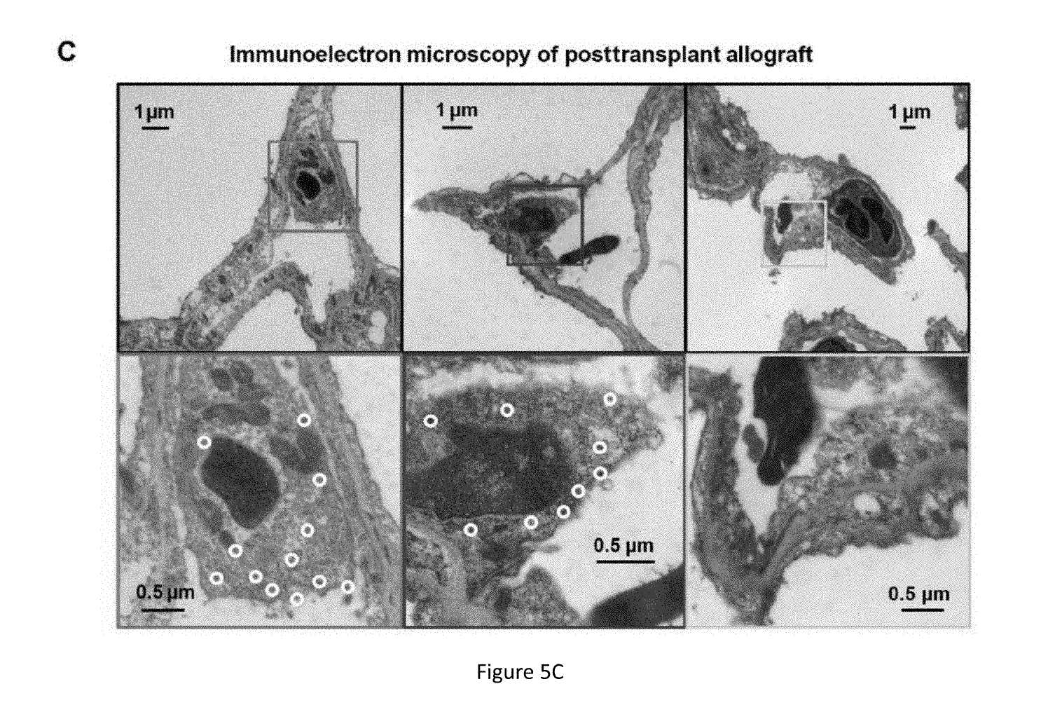

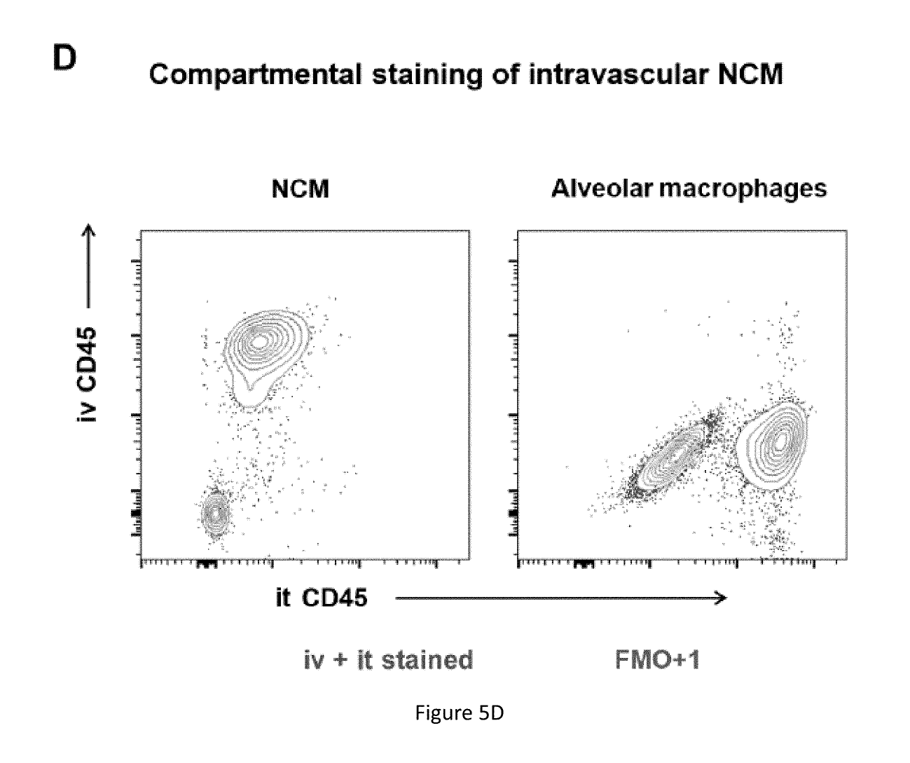

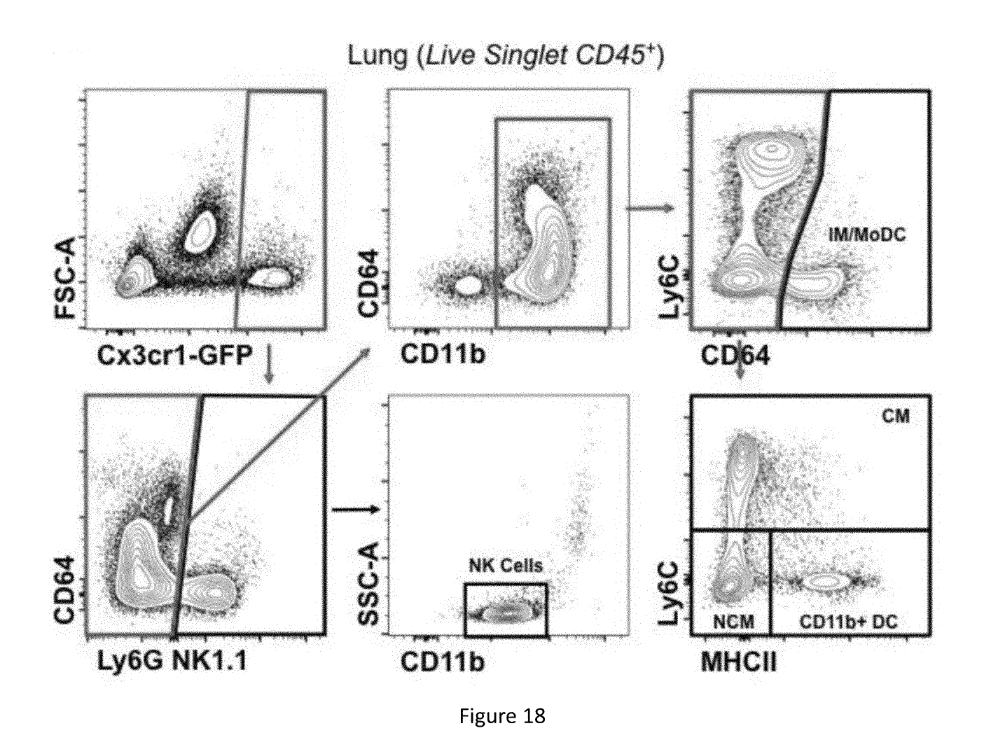

Pulmonary non-classical monocytes reside in the intravascular space. The selective and complete depletion of murine pulmonary NCM with clo-lip suggested that they are present only in the intravascular space under homeostasis. To confirm the anatomical localization of NCM in the lung tissue, we further studied the Cx3cr1^{gfp/+} reporter mouse. Two-photon imaging of murine Cx3cr1^{gfp/+} lungs revealed intravascular cells with green fluorescence (FIG. 5A). Additionally, there were interstitial GFP⁺ cells that resembled dendritic cells and macrophages. Flow cytometry revealed that GFP⁺ cells were predominantly comprised of NCM and classical monocytes (CM) along with a small fraction of dendritic cells, macrophages, and NK cells (FIG. 18). To further characterize the morphology of these cells, we evaluated the intravascular GFP⁺ cells in the lungs using electron microscopy with immunolabeling. Lungs were flushed to eliminate unbound intravascular cells from Cx3cr1^{gfp/+} mice and then labeled using gold nanoparticles conjugated to anti-GFP antibodies. Intravascular cells attached to the endothelium were found to have gold deposition and the morphology of mononuclear phagocytes, consistent with intravascular monocytes (FIG. 5B). Myeloid populations from flushed lungs were then sorted using flow cytometry and imaged. The in situ NCM resembled flow-sorted NCM, but not alveolar macrophages, classical monocytes, or natural killer cells (FIG. 5B). Specifically, pulmonary NCM were smaller than classical monocytes and larger than NK cells, and with a nucleus-to-cytoplasm ratio higher than classical monocytes but lower than NK cells. Both classical and NCM were rich in mitochondria, vacuoles, and endoplasmic reticulum, consistent with the known properties of monocytes as phagocytes and drivers of inflammatory responses, as previously described (18). We further imaged the Cx3cr1^{gfp/+} lung allografts using electron microscopy at 4 hours post-reperfusion to confirm the persistence of donor-derived NCM following ischemia-reperfusion. As shown in FIG. 5C, we found gold-labeled NCM bound to the endothelium near areas of exposed, thickened basement membrane and endothelial cell blebbing.

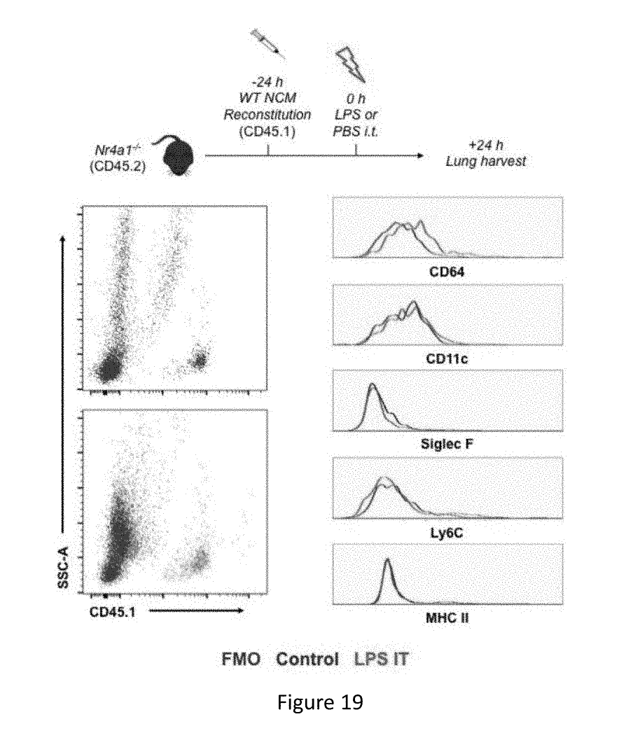

We then used intravenous and intratracheal compartmental staining (34, 35) to confirm that NCM in homeostatic lungs are in the intravascular space and to characterize their response to intratracheal lipopolysaccharide, a bacterial cell wall motif. All NCM stained with intravenously injected anti-CD45 antibodies but not with antibodies injected into the trachea (FIG. 5D). The intratracheal staining was effective as all alveolar macrophages stained for tracheally-administered anti-CD45 (FIG. 5D). Intratracheal lipopolysaccharide led to migration of CM, but not NCM, into the alveolar space (FIG. 5E). We then precluded the possibility of NCM differentiating into interstitial CM or alveolar macrophages following intratracheal lipopolysaccharide administration. Towards this, we adoptively transferred NCM isolated from wild-type B6-CD45.1 hosts into Nr4a1^{-/-} hosts (B6-CD45.2 background) and following engraftment of the NCM into the lungs, we challenged the reconstituted Nr4a1^{-/-} hosts with intratracheal lipopolysaccharide. None of the CM or alveolar macrophages were found to be CD45.1 positive confirming that NCM do not extravasate in response to intratracheal lipopolysaccharide (FIG. 19). Furthermore, treatment of wild type hosts with intravenous clo-lip did not affect neutrophil influx into the lungs following intratracheal administration of lipopolysaccharide (FIG. 5E), suggesting that depletion of NCM in the donor lungs might not impact recipient's defense against respiratory pathogens. Taken together, these findings indicate that NCM in native lungs exist in the intravascular space and while CM can move into the alveoli in response to intratracheal pathogen stimuli, NCM remain intravascular.

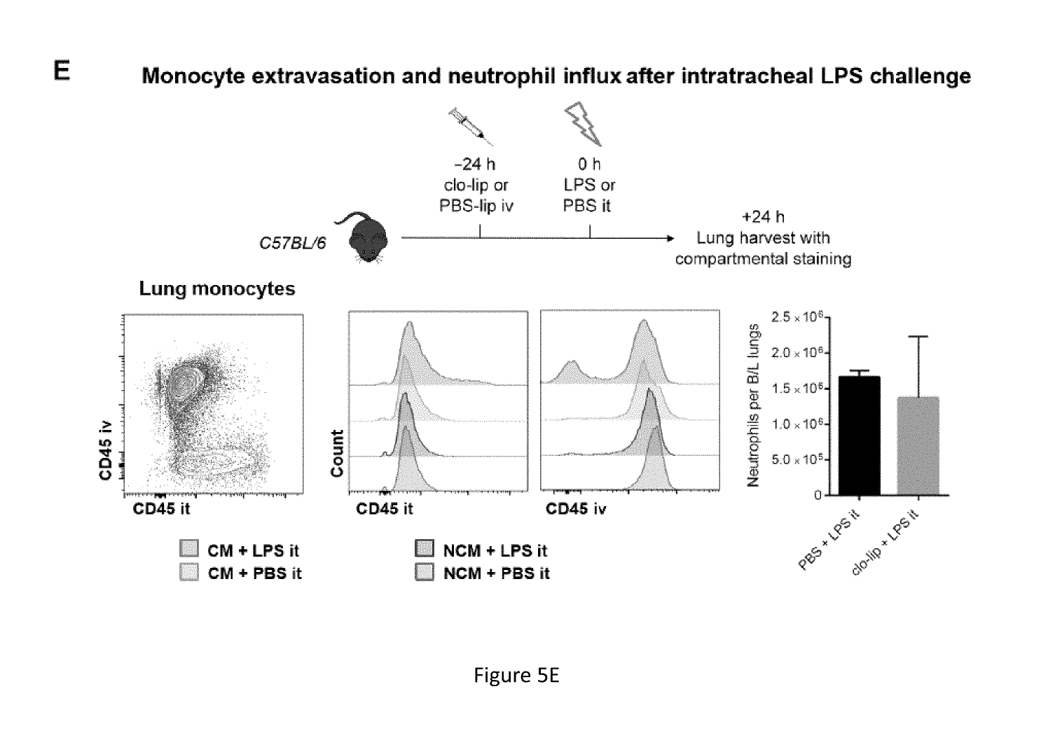

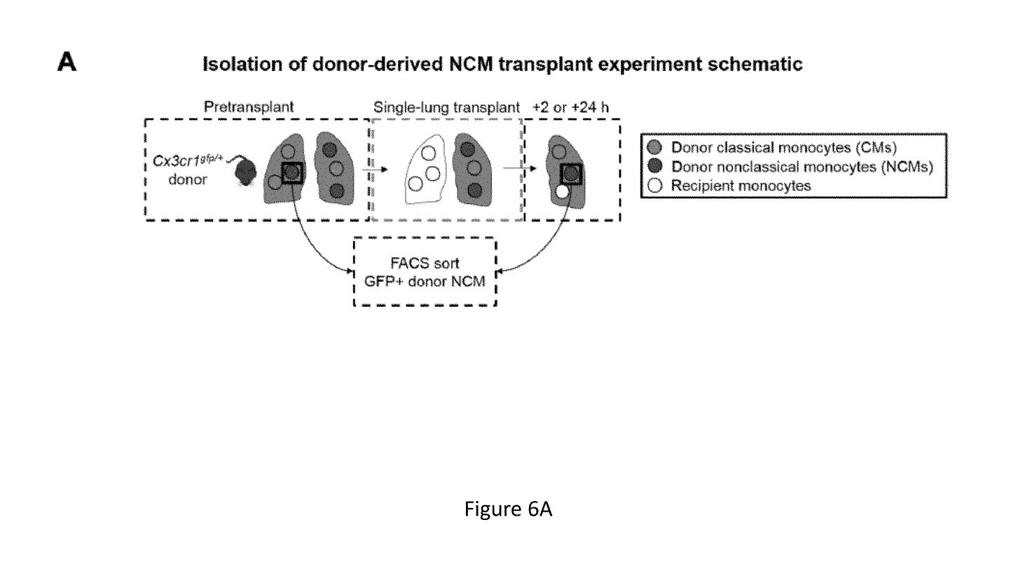

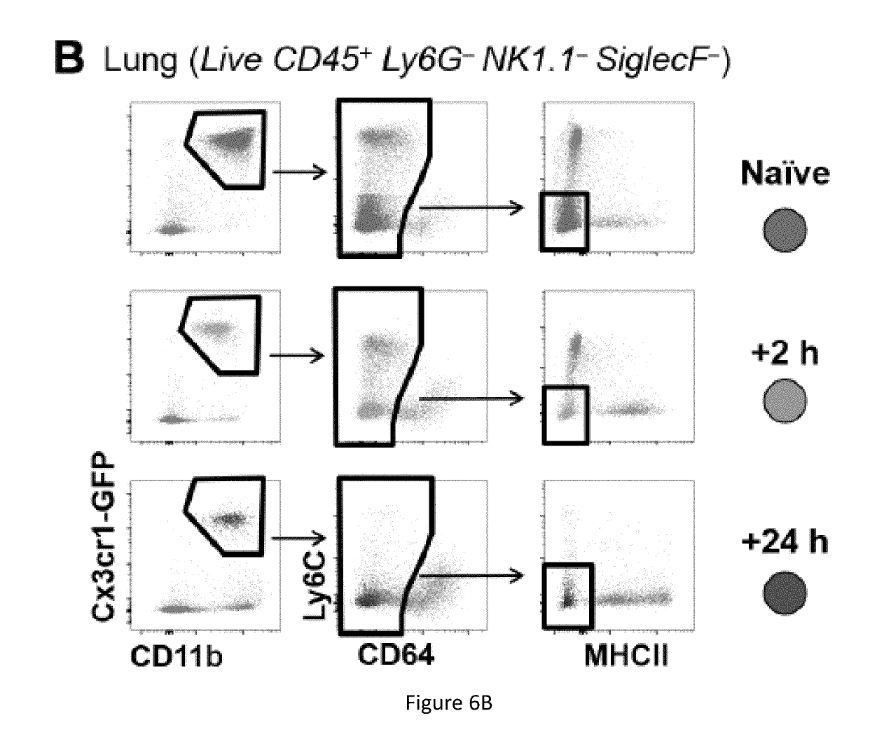

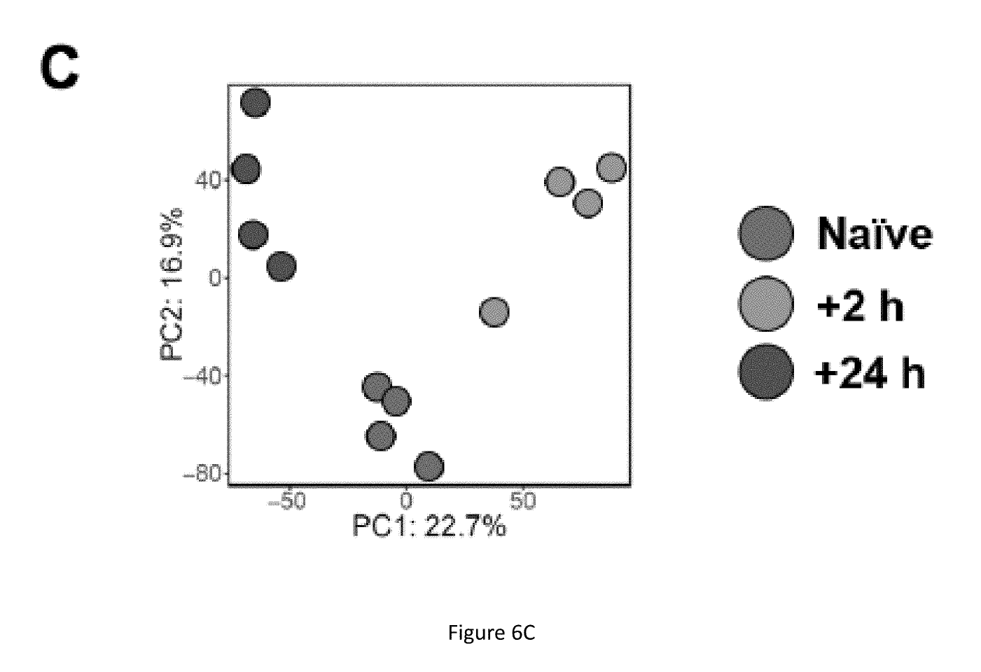

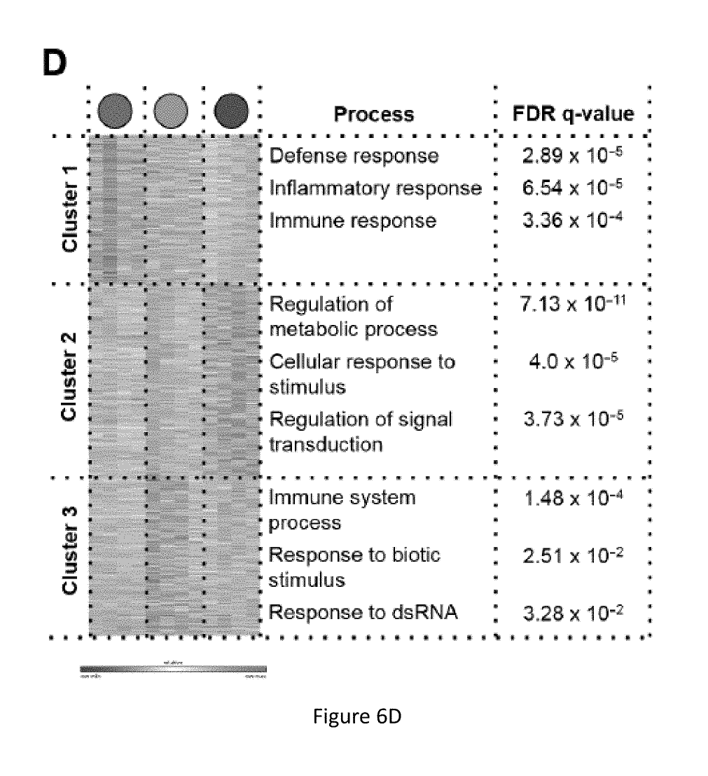

Transcriptomic profiling reveals upregulation of MyD88-pathway genes and neutrophil chemoattractants in donor NCM following reperfusion. Having confirmed that donor NCM were necessary and sufficient for recruiting neutrophils into the allograft, which leads to PGD, we then sought to investigate the mechanisms by which they recruited neutrophils. In order to do so, we utilized the Cx3cr1^{gfp/+} reporter mouse, which allowed for flow sorting of donor-origin NCM before and after allogeneic transplantation (FIGS. 6A&B). Donor-origin NCM isolated during the resting state of the donor and at different time points following transplantation underwent unbiased whole transcriptome analysis by RNA-Seq (FIGS. 6C&D). Given one prior report of NCM upregulating toll-like receptor (TLR) 7 transcripts and initiating neutrophil recruitment in a model of nephritis (18), we queried TLR pathways using KEGG PATHWAY mapping (http://www.kegg.jp/kegg/pathway.html). We found that genes from the TLR2/CD14, and MYD88 pathways were upregulated along with transcripts for macrophage inflammatory protein 2 (CXCL2 or MIP-2), a potent neutrophil chemoattractant (data not shown). Together, this suggested that TLR signaling and production of neutrophil chemoattractants, such as CXCL2, by donor NCM might be responsible for neutrophil recruitment following lung transplantation.

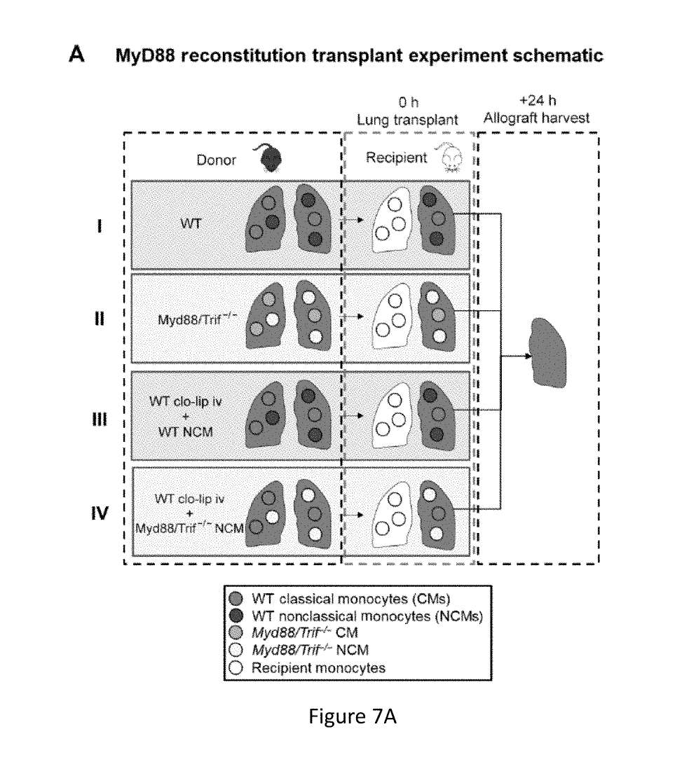

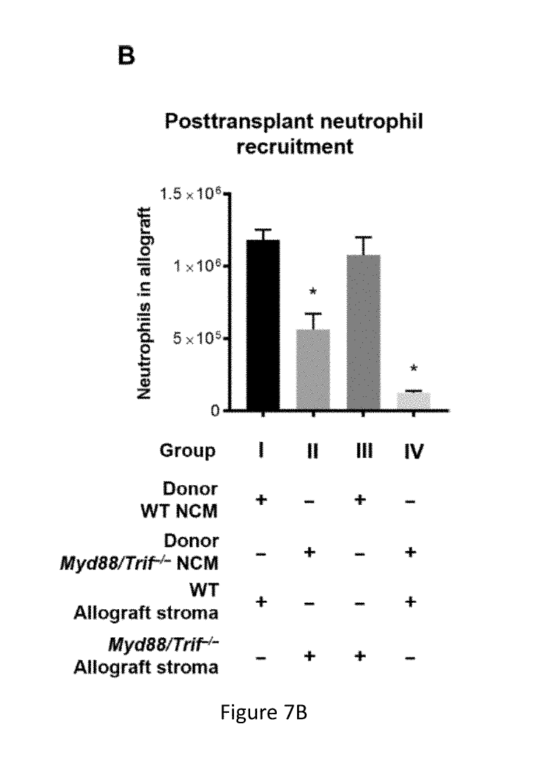

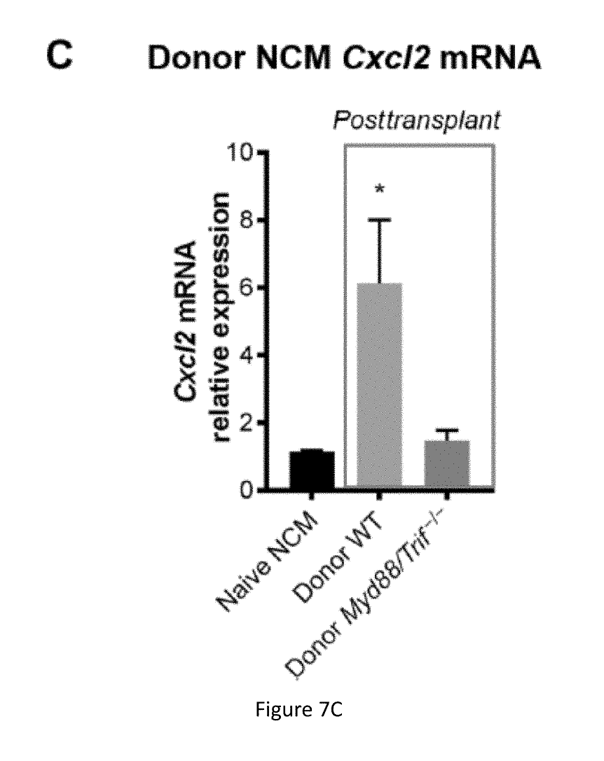

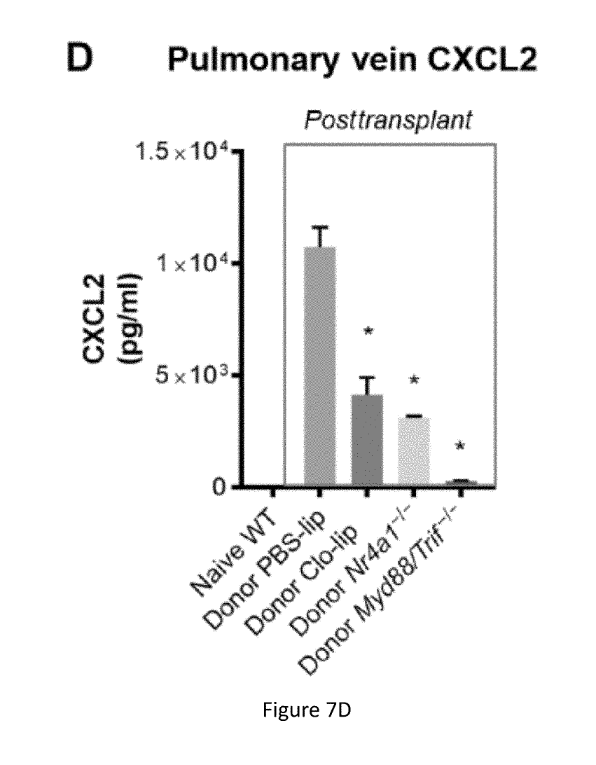

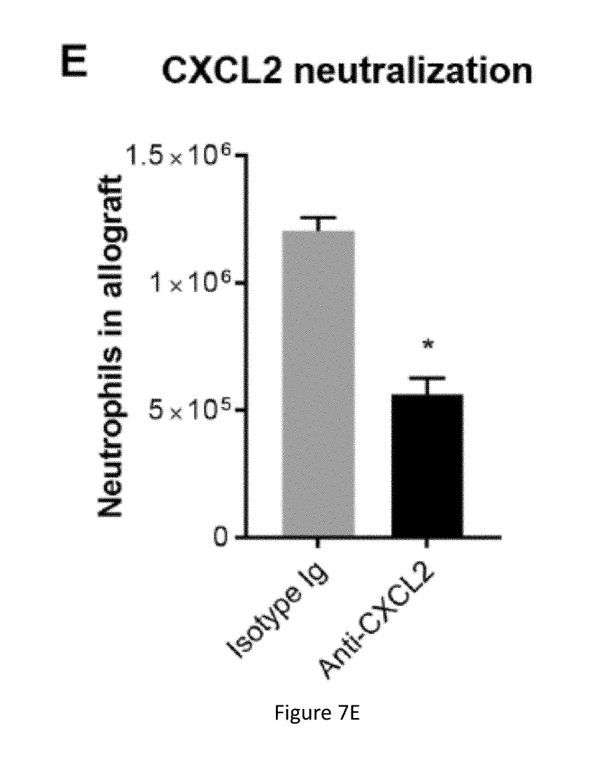

Donor non-classical monocytes produce CXCL2 in a TLR-dependent manner to recruit recipient neutrophils. All TLRs initiate their signaling cascade via their adaptor proteins MyD88 and/or TRIF. Supported by the data from our unbiased transcriptome analysis, we utilized the dual knockout Myd88^{-/-}/Trif^{-/-} mouse in a series of adoptive transfer experiments (FIG. 7A). When Myd88^{-/-}/Trif^{-/-} mice were used as donors, there was reduced neutrophil infiltration in the allograft compared with wild type donor lungs (FIG. 7B). These findings suggested that TLR signaling in the donor allograft, either in the stroma or the myeloid cells, was important for initiation of neutrophil recruitment. Indeed, prior studies have shown that TLR-dependent activation of stroma and endothelium can recruit monocytes and promote inflammation (18). However, the precise cell types and underlying mechanisms are unknown. To specifically investigate the role of TLR signaling in NCM, we reconstituted clo-lip treated donor lungs with NCM from either wild type or Myd88-Trif^{-/-} mice ex vivo following harvest and then utilized these reconstituted grafts in transplant (FIG. 7A). As shown in FIG. 7B, reconstitution with wild type, but not TLR-deficient NCM, restored neutrophil influx into the transplanted lungs indicating that TLR signaling in NCM is necessary for neutrophil recruitment.

CXCL2 has been shown to play an important role in neutrophil chemotaxis and extravasation in many tissues (36, 37) and has also emerged as a crucial chemokine in the recruitment of neutrophils to the lungs (9, 38-41). Given that Cxcl2 transcripts were upregulated in donor-derived NCM after transplant (data not shown), we tested whether MyD88-TRIF signaling was necessary for the production of CXCL2. We first transplanted either wild type or Myd88/Trif^{-/-} donor lungs into wild-type allogeneic recipients. Two hours after reperfusion, donor-derived NCM were isolated from the allograft and Cxcl2 mRNA transcript abundance was analyzed. We found that NCM from wild-type donors up-regulated Cxcl2, but NCM from Myd88/Trif^{-/-} donors did not (FIG. 7C). At the same time, analysis of allograft pulmonary vein blood showed increased CXCL2 levels following transplant of wild type donor lungs. However, CXCL2 levels were suppressed if NCM were depleted in donor lungs with clo-lip or if Nr4a1^{-/-} and Myd88/Trif^{-/-} donor lungs were used (FIG. 7D). Neutralizing CXCL2 using anti-CXCL2 antibodies at the time of transplantation, but not isotype control, attenuated neutrophil influx in NCM sufficient donors (FIG. 7E). Further, the allograft function was significantly improved in mice receiving anti-CXCL2 antibodies compared to isotype control (PaO₂ 469.7±27.3 vs 200.7±6.2 mmHg, p=0.0007). Collectively, these data indicate that NCM induce the recruitment of neutrophils by producing CXCL2 in a MyD88/TRIF-dependent fashion.

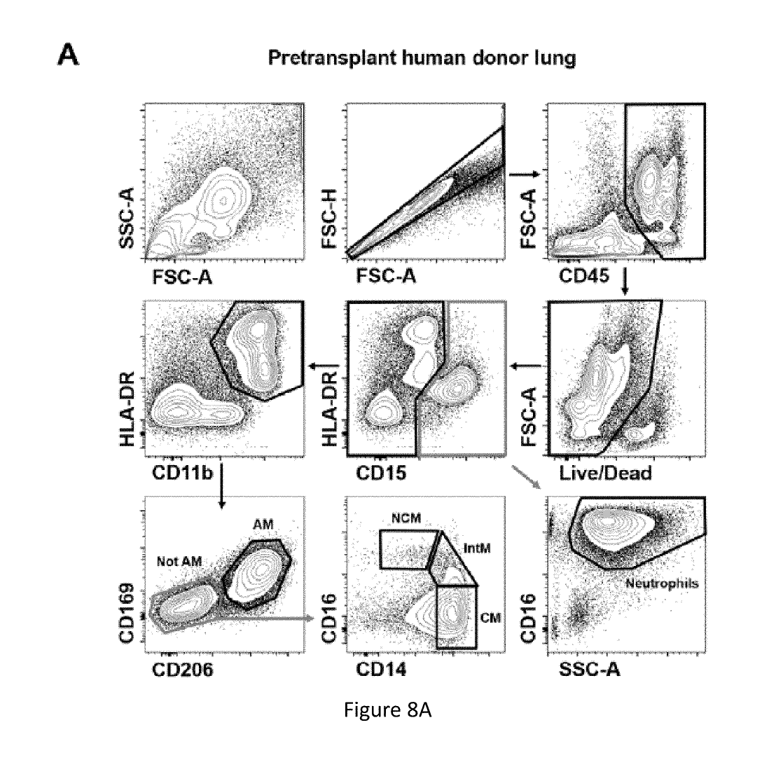

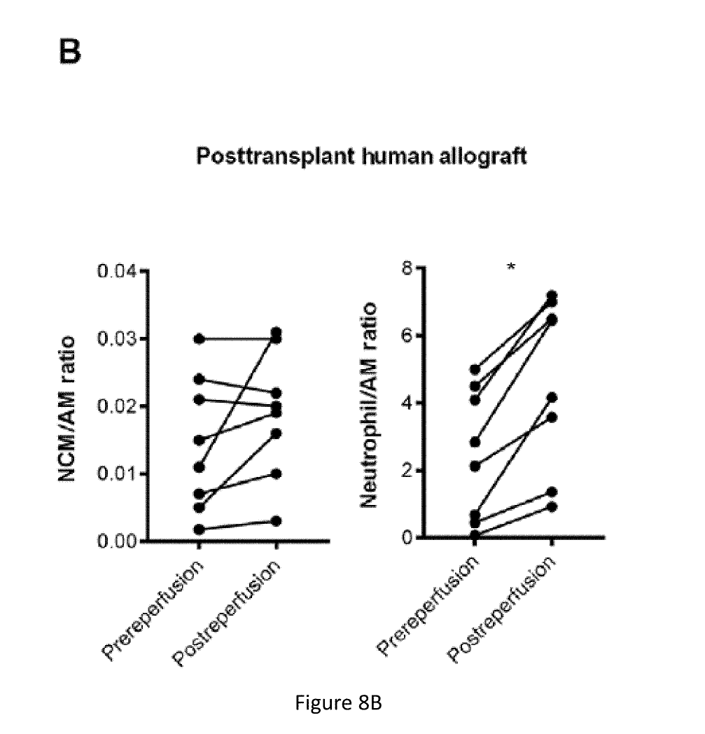

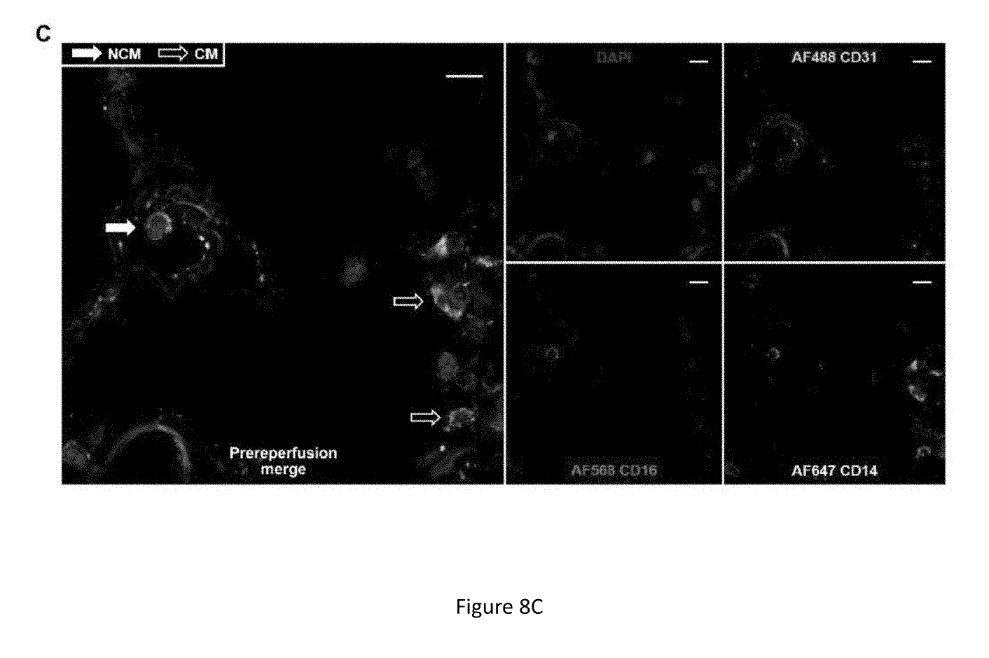

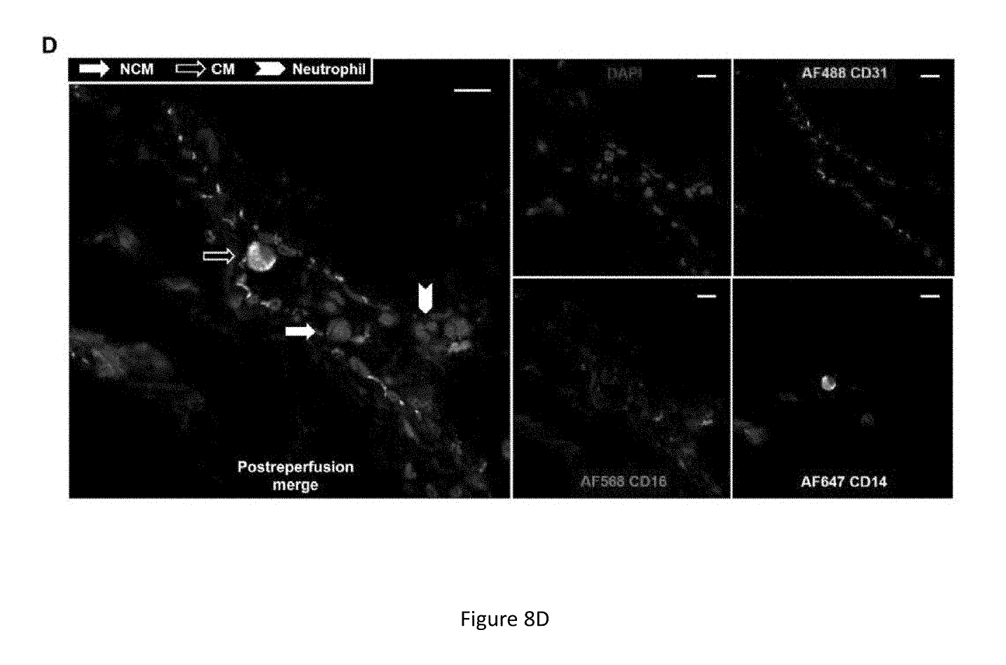

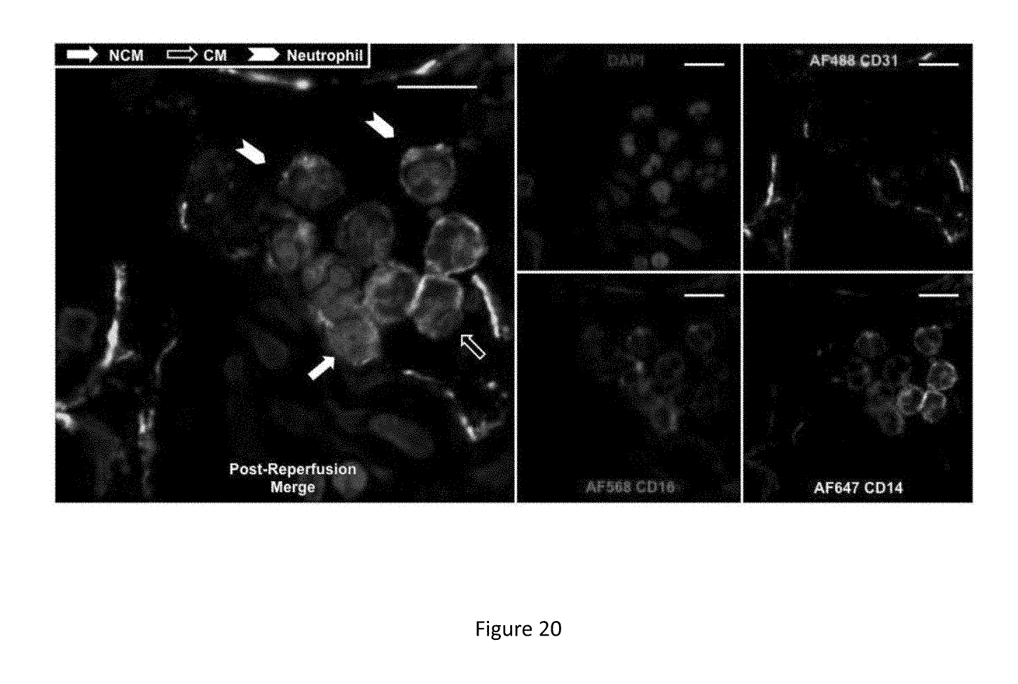

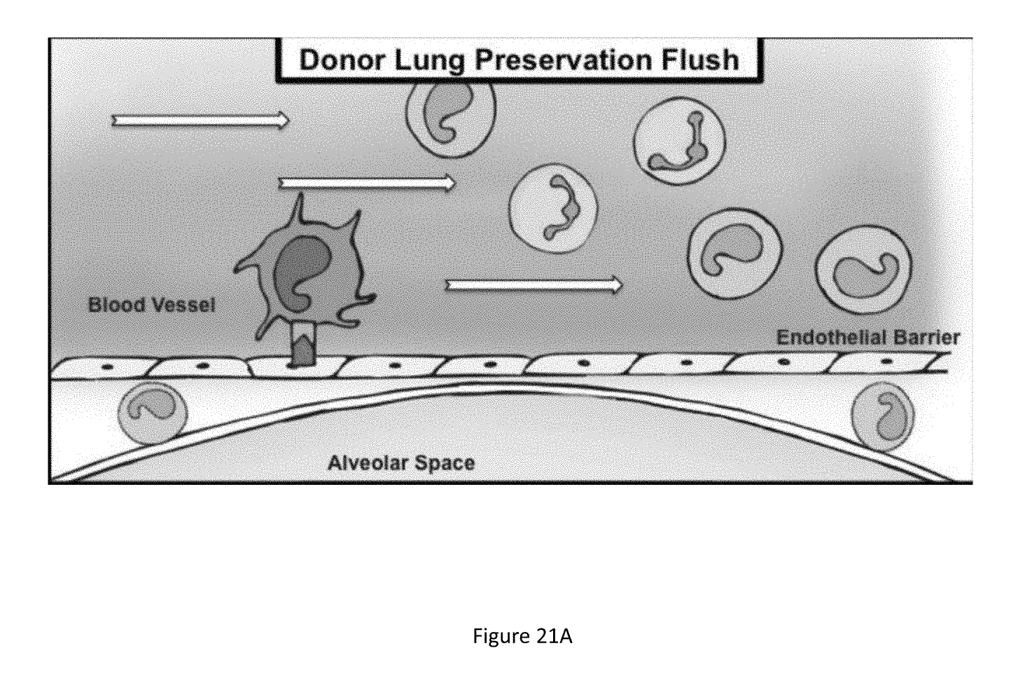

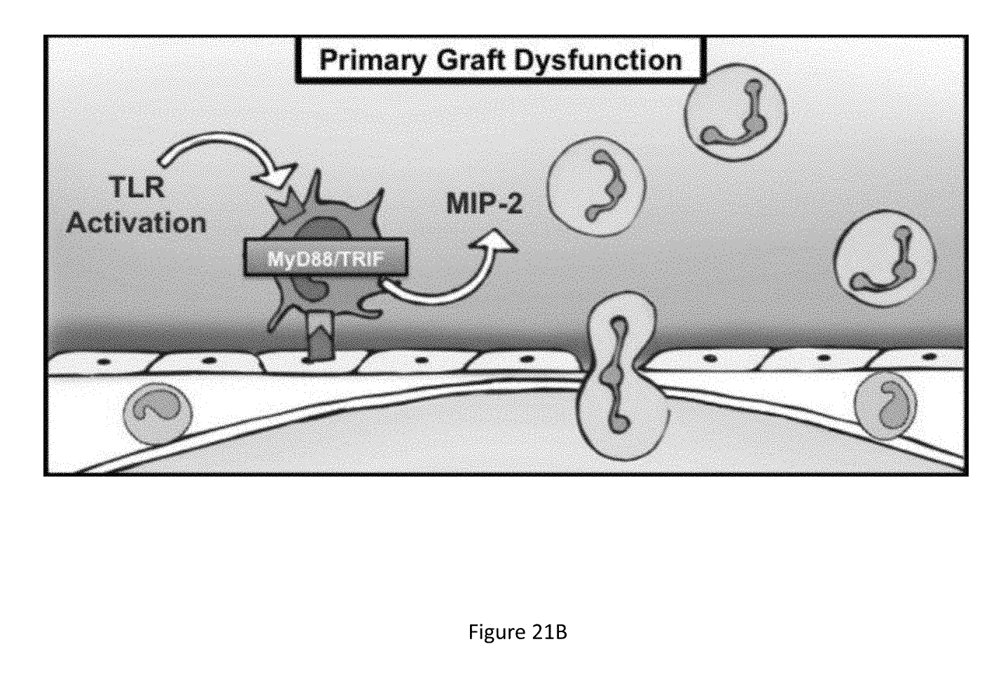

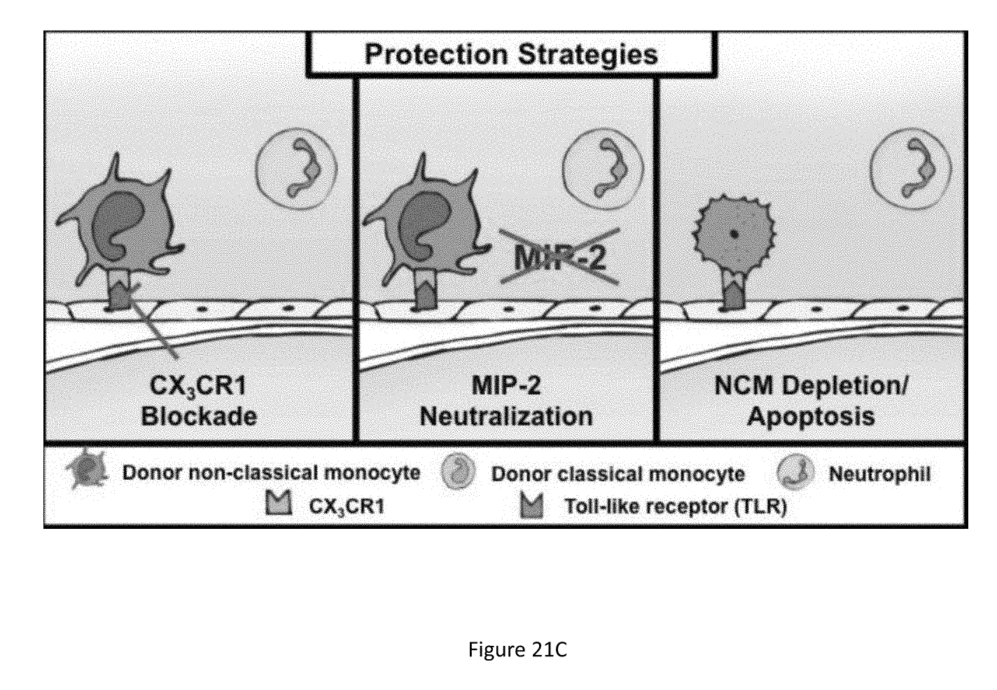

Non-classical monocytes persist in the vasculature of human donor lungs procured for transplantation. Ly6C^{low}CX3CR1^{hi}CCR2⁻ murine NCM are analogous to human CD14^{dim}CD16⁺⁺ monocytes (42). To determine whether our findings could apply to human lung transplantation, we evaluated the presence of NCM in human donor lungs procured for lung transplantation, using established protocols (22). These lungs were flushed using both antegrade and retrograde flush as is common in clinical practice (see Materials and Methods for details). CD45⁺CD15⁻HLADR⁺CD11b⁺CD169⁻CD206⁻ monocytes comprised 4-8% of resident lung myeloid cells. Of these, NCM (CD14^{dim}CD16⁺⁺) comprised 3-14%, classical monocytes (CD14⁺CD16³¹ ) comprised 62-83%, and intermediate monocytes (CD14⁺CD16⁺) comprised 12-19% (FIG. 8A). The population of NCM detected before reperfusion was stable at 90 minutes following reperfusion (FIG. 8B). Interestingly, there was a rapid increase in the number of neutrophils (FIG. 8B), similar to the murine allografts (FIG. 3). We used immunofluorescence of perfused human donor lungs and detected intravascular NCM (FIG. 8C). These cells persisted immediately after reperfusion (FIG. 8D) and were observed in the regions of neutrophil aggregation (FIG. 20). Together, these studies confirmed the presence of NCM in human donor lungs utilized in clinical transplant and verified the clinical relevance of our murine model.

Discussion

Here, we show that donor-derived intravascular pulmonary NCM are the primary drivers of neutrophil recruitment into the lung during ischemia reperfusion injury, and cause primary graft dysfunction (PGD) after lung transplant. These findings fundamentally change our understanding of the role of monocytes in the development of early ischemia-reperfusion injury and conclusively identify donor-derived NCM as the culprit myeloid cell. Genetic loss of TLR signaling in NCM through simultaneous deletion of MyD88 and TRIF prevented NCM-mediated neutrophil influx into the lung after transplantation, in part by preventing the release of the neutrophil chemokine CXCL2. Our studies of human lungs utilized in clinical transplantation confirmed that NCM persist in donor lungs and that there is a brisk neutrophil influx into the allograft, parallel to our murine model. Together, our data suggest that targeting NCM in the donor lung prior to transplantation might reduce the severity of PGD, the principal predictor of poor outcomes immediately following lung transplantation and the strongest risk factor for chronic allograft rejection (3, 4). Because these therapies could be applied to the donor lung prior to transplantation, it is unlikely they will be toxic to the recipient.

Distinct populations of circulating monocytes have been recently identified based on surface marker expression and morphology: classical and non-classical (13, 14, 43). Classical monocytes are a well-studied population of circulating bone marrow-derived cells that migrate into tissues in response to injury. In mice, these cells are identified by their high level expression of Ly6C, CD62L and CCR2, which are all involved in homing to sites of injury, and intermediate expression of CX3CR1 (43). At a steady state, classical monocytes differentiate into NCM, losing expression of Ly6C and CD62L while upregulating the expression of the fractalkine receptor CX3CR1 and CD43, a sialomucin involved in leukocyte adhesion (44). NCM adhere to the vascular wall where they “patrol” the vascular space, sometimes crawling against the flow of blood. The function of NCM in homeostasis and pathophysiology has been a recent topic of intense study, and in the context of inflammation, they have been shown to remove apoptotic endothelial cells and other vascular debris (43). Analogous populations of classical monocytes and NCM have been observed in humans distinguished by high and low expression of CD14, respectively (13, 14, 43).

In the context of lung transplantation, circulating classical monocytes in the recipient have been previously implicated in the development of PGD and models of ischemia-reperfusion injury such as hilar clamping (11). We believe that interpretations from these studies might have been confounded by lack of sufficient phenotypic markers to distinguish classical and non-classical monocytes and the inability to distinguish between vascular-adherent and circulating cells. In addition, it has been assumed that circulating monocytes are depleted from the donor lung through vigorous perfusion prior to transplant. We were therefore surprised to find NCM in biopsies from human lung allografts obtained immediately prior to transplant, a finding that was subsequently confirmed by other groups (22, 23). Using murine models, including the Cx3cr1^{gfp/+} reporter which allows for the tracking of functional monocytes (45), we were able to show that the population of intravascular monocytes retained in the lung after perfusion was exclusively composed of NCM and was completely depleted after the administration of clo-lip. Electron micrographs of the post-reperfusion lung showed that donor NCM were scattered throughout the vasculature and in areas of exposed basement membrane, suggesting that they mediated their effects either through a direct binding interaction with the vasculature or its underlying basement membrane, or perhaps through a paracrine release of chemokines or cytokines.

To determine whether NCM in the donor lung play a causal role in PGD, we depleted them prior to transplantation in our murine model. This resulted in a dramatic reduction in neutrophil egress into the alveolar space and a marked improvement in all of the physiologic markers of PGD. Interestingly, while we observed neutrophil egress into the alveolus in regions of the lung immediately adjacent to NCM during intravital imaging, neutrophil egress was also noted in regions where NCM were not present. This might result from limitations of currently available imaging techniques, or suggest a mechanism in which signaling in the NCM triggers the release of soluble chemokines or cytokines to affect generalized neutrophil egress. This latter hypothesis is consistent with our finding that NCM increased transcription of Mip2 after transplantation and that the administration of neutralizing anti-CXCL2markedly attenuated neutrophil influx after transplantation. This mechanism may also explain the dramatic change in phenotype we observed following depletion of the relatively rare population of NCM.

Previously, investigators attributed the reductions in neutrophil influx they observed in recipient mice treated with intravenous clo-lip immediately prior to transplantation to the depletion of classical monocytes in the recipient (11). However, we found this strategy also depletes donor-derived NCM in the allograft. By differentially administering cytotoxic antibodies directed against CCR2 (to selectively target pulmonary classical monocytes) and clo-lip (to target pulmonary NCM), we were able to show that the protection conferred by the administration of clo-lip to the recipient was attributable to the depletion of NCM in the donor. Indeed, depleting monocytes in the recipient without depleting NCM in the donor led to an unexpected increase in neutrophil recruitment into the lung. Ischemia-reperfusion injury causes PGD in over 50% of patients following human lung transplantation (1-4), but not all recipients experience PGD. We postulate that some of the heterogeneity in the development of PGD following human lung transplant could be explained by the number or viability of NCM in the donor lung at the time of transplantation, and perhaps the number and function of classical monocytes in the recipient. Using fate-mapping techniques, prior studies have indicated that the lifespan of NCM is about 2 days (46). However, if classical monocytes are depleted, NCM survive for up to 5 days (46). Hence, variability in the number of NCM and their ability to survive and perpetuate injury within donor lungs might affect development of human PGD.

The orphan nuclear receptor NR4A1 has been shown to be required for the differentiation of classical monocytes into NCM (32). NR4A1-deficient mice lack NCM in the circulation and tissues and therefore provide a genetic approach to determine the importance of donor-derived NCM in the development of PGD. We found that neutrophil influx into the engrafted lung after transplant and the severity of the resulting PGD was dramatically reduced when lungs from Nr4a1^{-/-} donors were transplanted into wild type mice. This protection is not attributable to a function of NR4A1 independent of its inhibition of NCM differentiation, as the adoptive transfer of functional NCM into Nr4a1^{-/-} mice prior to transplant restored neutrophil influx into the allograft. Further genetic evidence supporting the importance of NCM in neutrophil influx comes from mice lacking the fractalkine receptor CX3CR1. In some models, CXC3CR1 has been suggested to be required for the patrolling behavior of NCM along the vascular endothelium while in a kidney injury model, it was shown to be required for neutrophil influx independent of any effect on their patrolling behavior (20, 47, 48). Similar to NR4A1 deletion, we found that donor lungs from mice deficient in CX3CR1 showed marked attenuation of neutrophil influx following transplantation. Our findings did not suggest a role for CX3CR1 in the adherence of NCM to the pulmonary vasculature as the number of NCM in donor lungs from Cx3cr1^{gfp/gfp} mice was similar to controls. While it could be argued that in our pharmacologic depletion studies that donor lungs retained clodronate liposomes and depleted recipient blood monocytes upon reperfusion, clodronate liposomes do not cross capillary barriers and are not known to adhere to pulmonary vasculature (49) making this an unlikely scenario. Additionally, we found that genetic deletion of either CX3CR1 or NR4A1 in the donor lung prevented neutrophil influx into the lung after transplantation, and reconstitution of either clodronate depleted or Nr4a1^{-/-} donor lungs with flow-sorted NCM from wild-type mice restored neutrophil influx. These data demonstrate that donor-derived pulmonary NCM are necessary and sufficient for initiating neutrophil influx after lung transplantation independent of potential off-target effects of clo-lip depletion.

Our group and others have previously shown that neutrophil recruitment, in certain models of inflammation, including the serum-induced model of rheumatoid arthritis and a model of autoimmune kidney injury, is dependent on NCM which may be activated through

TLR7 ligation (18, 50). Guided by these previous reports, we examined the role of TLR signaling in NCM in mediating the egress of neutrophils during ischemia reperfusion injury. We indeed found that transcripts for genes involved in TLR-signaling through its downstream adaptors were up-regulated, but interestingly found that in this model, TLR2 and its coreceptor CD14 were up-regulated, while TLR7 was not. In confirmatory studies, we used mice doubly deficient in MyD88 and TRIF. One or both of these proteins is required for signaling through all of the described TLRs (51). We found that transplantation of lungs from these mice into wild type recipients reduced neutrophil egress into the lung after transplantation to a level similar to that observed in allografts depleted of NCM. Furthermore, reconstitution of wild type clo-lip treated donor lungs with flow-sorted NCM from MyD88-Trif^{-/-} failed to restore neutrophil recruitment into the lung, while reconstitution using NCM from wild type mice did. NCM, however, did not mediate neutrophil influx in response to lipopolysaccharide, suggesting that they are not activated through TLR4 but a damage-associated molecular pattern-sensing pathway. Of note, our studies of NCM depletion using pre-treatment with clo-lip prior to lipopolysaccharide challenge differ from previous studies that utilized clo-lip to induce depletion of circulating monocytes after lipopolysaccharide administration (49). Hence, pulmonary intravascular NCM may play a broader role in mediating sterile, but not pathogen-induced, lung inflammation and their transient depletion may not compromise host pathogen defense.

Our studies identify non-classical monocytes as the causal cell type responsible for ischemia reperfusion injury through the production of neutrophil chemoattractants. One of these, CXCL2, has been shown to play an important role in neutrophil recruitment (38-41). Unbiased whole transcriptome analysis and confirmatory qPCR showed that Cxcl2 transcripts were upregulated nearly 10-fold following graft reperfusion. Accordingly, we examined the expression of Cxcl2in flow-sorted NCM from wild type and MyD88/Trif^{-/-} donor mice after transplantation and found that Cxcl2 mRNA was markedly elevated post-transplant in the wild type but not Myd88^{-/-}/Trif^{-/-} donor NCM. This could represent a scenario in which stored CXCL2 is immediately released upon activation and the observed transcription is occurring in response to replenish stores and continually produce the chemokine due to ongoing TLR-signaling. Consistent with this hypothesis, parallel differences were observed in the levels of CXCL2 in the pulmonary veins from the donor lungs after transplant.

We show that the strategies currently used to perfuse lung allografts leave a population of NCM in the vasculature of the human lung. In mice, this retained population is sufficient to mediate neutrophil egress into the lung after transplantation. These findings have important potential clinical applications. Bisphosphonates like clodronate have a long history of safety in humans (33) and pharmacologic procedures to encapsulate drugs into liposomes that facilitate uptake by macrophages can be made readily available. While the effects of these medications may preclude their administration to the recipient in the perioperative period after transplant, administration of an NCM-depleting agent to the donor lung at the time of procurement or ex vivo prior to implantation is predicted to be safe and feasible. Safety might be further improved through strategies that detach NCM from the vasculature without killing them, allowing them to be efficiently flushed from the pulmonary vasculature and may transiently prevent the recruitment of recipient-derived NCM after implantation. Alternatively, the activation of NCM in the donor lung might be inhibited through the use of TLR-antagonists. Finally, chemokines or cytokines released from NCM after transplantation might be targeted in the recipient, which could dampen neutrophil recruitment by recipient derived NCM recruited to the allograft after transplantation, but would have to be modulated closely to prevent adverse effects of such a strategy.

Taken together, these findings represent a significant advance in our understanding of the role of monocytes during inflammation and a fundamental change in our comprehension of the pathophysiology of ischemia-reperfusion injury and PGD with important clinical implications. Our findings in a murine lung transplant model suggest that targeting these NCM in the donor lung prior to transplantation will likely reduce the disease burden of primary graft dysfunction following lung transplantation.

Materials and Methods

Study Design. The objective of this study was to determine the role of donor-derived nonclassical monocytes in the pathogenesis of primary lung allograft dysfunction. Flow cytometry and immunofluorescence microscopy were used to identify nonclassical monocytes in donor human lungs before and after reperfusion. Murine allogeneic single lung transplant was used as a model of human transplantation and to test the effects of donor and recipient treatments, genetic deletion, and cell adoptive transfer. Intravital two-photon microscopy and flow cytometry were used to measure the influx of inflammatory cell populations into the lung. Compartmental staining for flow cytometry, immunofluorescence two photon microscopy, and immuno-electron micoroscopy were used to anatomically localize nonclassical monocytes in the intravascular space. To identify activation pathways and proinflammatory chemokines released by nonclassical monocytes, RNAseq was performed on FACS sorted donor-derived nonclassical monocytes in the allograft early after transplant. Genetic deletion of MYD88 and TRIF and measurement of CXCL2 in the post-transplant allograft were performed based on their identification as candidate activation pathways and as an identified neutrophil chemokine during analysis of whole transcriptome data. In an independent set of experiments, to examine the effects of nonclassical monocytes on the host's ability to respond to pathogen, mice depleted of nonclassical monocytes were treated with intratracheal instillation of lipopolysaccharide to model gram negative organism-induced lung injury. In vivo experiments represent pooled results of at least two repeated experiments unless otherwise indicated. Details of all protocols are provided below.

Human Donor Lungs. Lungs for human transplantation were obtained from donors who met standard donation criteria (52). The lungs were procured in a standardized fashion by first perfusing the main pulmonary artery with a low-potassium dextran (Perfadex) solution cooled to 4oC (XVIVO Perfusion) at a volume of 60 ml/kg (53). Additionally, the lungs were perfused in a retrograde manner with 250 ml of the same solution through each of the four pulmonary veins. The lungs were transported for transplantation at 4oC. A 1×1 cm piece of lingula or right middle lobe was serially resected immediately after removing the lungs from cold storage, and 90 minutes following reperfusion. To achieve consistency, the serial biopsies of human lungs were obtained from the exact same region of the donor pre- and post-reperfusion. Lung specimens were processed immediately as recently described (22). Since alveolar macrophages are the most consistent cell population throughout the lung before and after transplantation (54), cell counts were expressed as a ratio per AM to achieve meaningful comparison. Human lung samples were acquired in concordance with the Northwestern University Institutional Review Board policies and regulations.

Animals and Reagents. The mice used were 10-16 weeks and weighed 24-30 g. Wild type C57BL/6 (B6), BALB/c (B/c), Cx3cr1/gfp/gfp, Nr4a1−/−, congenic CD45.1 and CD45.2 mice were commercially acquired (Jackson Laboratories). Myd88−/− and Trif−/− mice were crossed to develop Myd88/Trif−/− mice on the B6 background. The mice were housed at Northwestern University Animal Care center, in a specific pathogen-free environment with climate controlled rooms and free access to standard pelleted food and water. All experiments using mice were performed in accordance with protocols that were approved by Northwestern University Institutional Care and Use Committee. Clodronate-loaded liposomes and control phosphate-buffered saline liposomes were purchased (Clodroliposomes). At specific time points as discussed in the results, mice were injected intravenously with 200 ul of clodronate-loaded liposomes or control PBS-loaded liposomes (25). Cytotoxic anti-CCR2 antibodies were a kind gift of Steffen Jung and used for selective depletion of CCR2+ classical monocytes as previously described (45). Purified anti-CXCL2 antibodies (R&D Systems) and anti-CX3CR1 antibodies (Torrey Pines Biolabs) were commercially acquired. Multiplex cytokine arrays were performed according to standardized manufacturer's instructions (Luminex Multiplex Assays, ThermoFisher Scientific).

Murine lung transplantation. Orthotopic murine left lung transplantation was performed as previously described (27, 55). Donor mice were anesthetized with a mixture of xylazine (10 mg/kg) and ketamine (100 mg/kg). The donor lungs were flushed with 3 ml of preservative solution through the pulmonary artery. The heart-lung block was excised and then stored in cooled (4oC) preservative solution. The bronchus, pulmonary vein, and artery were dissected free and prepared for anastomosis. A customized cuff made of a Teflon intravenous catheter applied to the vascular structures and fixated with a 10-0 nylon ligature. After placement of a microvessel clip on the bronchus to avoid airway infiltration with preservative solution, the graft was stored at 4oC for a period of 90-120 minutes of cold ischemic time prior to implantation. The recipient mice received subcutaneous buprenorphine (0.1 mg/kg) 30 min prior to incision and every 6 hours as needed after the procedure. The recipient mice were intubated and a left-sided thoracotomy was performed within the third intercostal space. The recipient's native lung was gently clamped and pulled out of the thoracic cavity. The space between the artery, the vein and the bronchus was dissected separately. The artery and vein were temporarily occluded using 8-0 nylon ligatures. The anastomoses were completed by fixating each cuff with 10-0 nylon ligatures. The occlusion ligatures were released (first vein, then artery) and the lung inflated. The chest incision was closed and recipients separated from the ventilator when spontaneous respiration resumed. No antibiotics or immunosuppressive agents were used postoperatively in any groups unless otherwise noted. For reconstitution of clodronate-loaded liposome treated donors, we used 5×105 freshly sorted lung NCM from either wild type or Myd88/Trif−/− mice. These NCM were injected into the pulmonary artery of the donor murine lungs ex vivo, immediately prior to implantation of the donor lung. For reconstitution of Nr4a1−/− mice, freshly sorted NCM from Cx3cr1gfp/+ mice were used and injected intravenously through a retro-orbital injection.

Assessment of Lung injury. To obtain arterial oxygenation measurements, mice were intubated, mechanically ventilated on 100% oxygen, and a sternotomy was performed. The right lung hilum was clamped for 5 minutes, then 100 ul of arterial blood was drawn from the aorta via a heparinized syringe and immediately analyzed using an i-STAT blood gas analyzer (Abbott). Graft function was expressed as the partial pressure of arterial oxygen (PaO2) while on 100% inhaled oxygen (FiO2). For histological analyses, the whole lung was harvested and gently flushed through the pulmonary artery with 3ml saline. 4% paraformaldehyde was instilled into the trachea with a pressure of 10 cmH2O then fixed for 48-72 hours prior to being embedded in paraffin. The whole lung was serially sectioned and stained with hematoxylin and eosin (H&E). Evans blue dye extravasation and wet-dry ratio of the lungs were performed as previously described (29).

Multicolor flow cytometry. Single cell suspensions from mouse whole lung and human lung wedge biopsies were obtained as previously described (22, 56). Murine peripheral blood was storage in EDTA-coated tubes. Whole blood was utilized for staining, after which simultaneous cell fixation and red blood cell lysis was performed utilizing FACSLyse (BD Biosciences). Antibodies utilized for murine cell staining included rat-anti mouse CD24-BUV395 (M1/69, BD), rat anti-mouse CD45-FITC (30-F11, BioLegend), CD45.1-FITC (A20, BD), rat anti-mouse CD11b-FITC (M1/70, BD), rat anti-mouse I-A/I-E-FITC (M5/114.15.2, BioLegend), rat anti-mouse Ly6C-eFluor450 (HK1.4, eBiosciences), rat anti-mouse I-A/I-E-PerCPCy5.5 (M5/114.15.2, BioLegend), rat anti-mouse CD24-APC (M1/69, eBiosciences), rat anti-mouse CD45-APC (30-F11, BioLegend), rat anti-mouse CD45.2-APC (104, BD) rat anti-mouse CD3-APC (145-2C11, eBiosciences), rat anti-mouse Ly6G-AlexaFluor 700 (1A8, BioLegend), rat anti-mouse NK1.1-AlexaFluor 700 (PK136, BD), rat anti-mouse CD11b-APCCy7 (M1/70, BioLegend), rat anti-mouse CD64-PE (X54-5/7.1, BioLegend), rat anti-mouse CD115-PE (AFS98, eBiosciences), rat anti-mouse SiglecF-PECF594 (E50-2440, BD), rat anti-mouse CD19-PECF594, (1D3, BD), rat anti-mouse F4/80-APC (BM8, eBiosciences), rat anti-mouse CD11c-PECy7 (HL3, BD), rat anti-mouse CD62L-PECy7 (MEL-14, eBiosciences).

Antibodies utilized for human cell staining included mouse-anti human CD45-BB515 (H130, BD), mouse-anti human CD14-PerCPCy5.5 (M5E2, BD), mouse anti-human HLA-DR-eFluor450 (L243, eBioscience), mouse anti-human CD169-APC (7-239, Biolegend), mouse anti-human CD15-AlexaFlour 700 (W6D3, Biolegend), rat anti-mouse/human CD11b-APCCy7 (M1/70, Biolegend), mouse anti-human CD16-PE (3G8, BD), mouse anti-human CD163-PECF594 (GHI/61, BD), mouse anti-human CD206-PECy7 (19.2, eBioscience). Fixed samples were run on acustom LSRFortessa Cell Analyzer (BD) for flow cytometry analysis. Fresh samples were sorted using a FACSAria (BD). Acquired data was analyzed with FlowJo v10 (FlowJo).

Two-photon Microscopy. For imaging of fresh lung explants, 100 ul of a 1:4 dilution of Qtracker 655 vascular (Life Technologies) was injected intravenously and allowed to circulate for 3 minutes prior to sacrifice. Following animal sacrifice, tracheostomy was performed and the anterior chest wall was removed. 1 mL of 30% sucrose was instilled via the tracheostomy, then and the right and left hila were ligated with silk suture and hilar structures transected proximal to the suture ligature. The resulting lung block thus retained alveolar inflation and contained Qtracker655 to label blood vessels. This lung block was then attached to the underside of a microscope slide cover utilizing a ring of VetBond (3M) and immersed in ice cold sterile PBS. Imaging was performed utilizing a water immersion lens on an A1R-MP+ multiphoton microscope system with a Coherent Ti:S Chameleon Vision S laser tuned to 890 nm. The post-transplant intravital two-photon microscopy was performed as previously described in our previous report (11).

Immuno-Electron Microscopy. After exposing the heart and bilateral lungs, the intrathoracic inferior vena cava and aorta were transected. The right ventricle was flushed with 5 mL of HBSS, followed by 5 mL of fixative composed of 4% paraformaldehyde and 0.1% gluteraldehyde on 0.1 M cacodylate buffer. The bilateral lung block was then gently instilled with 1 mL of fixative solution to re-recruit the alveoli and fixed in 10 mL of fixative buffer overnight at 4° C. After overnight fixation, the pleura was stripped and the subpleural parenchyma minced gently into 1-2 mm pieces and replaced in fixative buffer and kept at 4° C. After dehydration in a graded series of ethanol, the pieces were embedded in LRWhite embedding medium (EMS) cured at 58° C. overnight. Ultra-thin sections were collected on nickel grids and were blocked with 1% BSA in PBS and primary staining performed with a 1:20 dilution of polyclonal rabbit anti-GFP (ab6556, Abcam) followed by 10 nm immunogold goat anti-rabbit IgG (Jackson Immunoresearch). The grids were contrasted with 3% aqueous uranyl acetate and Reynold's lead citrate solutions and dried. Sorted cells were fixed in the same fixative and mixed with 10% gelatin, then post-fixed with 1% osmium tetroxide, dehydrated in a graded series of ethanol and embedded in Epon812 (EMS). Ultrathin sections of cells were also contrasted with 3% aqueous uranyl acetate solution and Reynold's lead citrate, and dried. Samples were imaged using a FEI Tecnai Spirit G2 transmission electron microscope (FEI Company) operated at 80 kV. Images were captured by Eagle 4 k HR 200 kV CCD camera.

Compartmental Lung Intravenous and Intratracheal Staining. Intravenous and intratracheal was performed utilizing an adaptation of previously described methods (34). 6 ug of APC-conjugated anti-CD45 in 100 ul of sterile PBS was injected IV and allowed to circulate for 3 minutes prior to euthanasia with an overdose of Euthasol. Tracheostomy was performed. The vena cava was transected and the right ventricle flushed with 10mL of HBSS to wash unbound IV antibody. The heart was removed and the bilateral lung block was then gently instilled with lug of FITC-conjugated anti-CD45 mAb in 1 mL of HBSS and incubated at room temperature for 5 minutes. The instilled antibody-containing volume was then removed and 3 sequential 1 mL bronchoalveolar lavage (BAL) washes were performed and collected to remove any unbound IT antibody prior to enzymatic digestion and mechanical dissociation of the whole lung. The collected BAL was washed with 10 mL of MACS buffer, pelleted, re-suspended, and passed through a 40 um filter and combined with the single cell suspension from the whole lung digest to proceed with ex vivo staining.

RNA sequencing. NCM were sorted from naïve, 2 hour, and 24 hour post-perfusion Cx3cr1gfp/+ lungs according to the gating strategy shown in FIG. 6B. Samples with less than 10,000 cells were sorted directly into extraction buffer then thoroughly mixed. Total RNA was extracted using the Arcturus PicoPure RNA Isolation Kit (ThermoFisher) with an average yield of 18 ng per naïve sample and 2.8 ng per post-perfusion sample. Total RNA with an RIN>7.0 (average 8.7) was then preamplified and cDNA synthesis was performed using the SMART-Seq v4 Ultra Low Input RNA Kit for Sequencing (Clontech, Takara Bio USA). Libraries were then prepared using the Nextera XT DNA Library Preparation Kit (Illumina), indexed using the Nextera Index Kit v2 (Illumina), and sequenced on a NextSeq500 (Illumina). FASTQ files were generated, then single-end reads mapped to the mouse reference genome mm10 using TopHat2 aligner software with the HTSeq package resulting in an average of 74.7% (range 72.6-77.4%) singly-mapped reads per sample and an average of 18.5×106 (range 10.1-29.4×106) singly-mapped reads per sample. Raw counts were used for a differential gene expression analysis using edgeR (R version 3.2.3) with an adjusted p<0.05 by pair-wise comparisons to identify differentially expressed genes (DEG). A filter for genes having a count per million (cpm) sum of at least 2 including samples with at least 1 cpm was done to eliminate low expressing and non-expressing genes before DEG analysis. Principle component analysis (PCA) of samples was done using log 2CPM (count per million reads) values of all detected genes in R. Heat maps were generated using K-means clustering with K=3 using GENE-E (Broad Institute), gene ontology analysis was performed using Gorilla Gene Ontology (57), and pathway analysis done using KEGG Pathways (www.kegg.jp/kegg/pathway.html).

MIP-2 analysis. Total RNA was extracted from sorted cells or pulmonary vein blood with Trizol reagent following the manufacturer's instructions (Invitrogen). cDNA was generated from lug RNA using superscript III reverse transcriptase (Invitrogen) following the manufacturer's instruction. Real-time PCR amplification and analysis was used the 7900HT real-time PCR system (Applied Biosystems). The relative amount of gene was normalized to B-Actin expression. MIP-2 ELISA was performed using a commercially available kit accordingly to manufacturer's instructions (R&D Systems, Minneapolis, Minn.).

Statistical Analysis. All data were presented as mean±standard error of mean (SEM). Comparison between two groups was performed by unpaired ‘t’ tests with Sidak-Holm correction for multiple comparisons, unless otherwise noted in the figure legends.

### REFERENCES

1. M. K. Porteous, J. M. Diamond, J. D. Christie, Primary graft dysfunction: lessons learned about the first 72 h after lung transplantation. Curr Opin Organ Transplant 20, 506-514 (2015).

2. J. D. Christie, M. Carby, R. Bag, P. Corris, M. Hertz, D. Weill, I. W. G. o. P. L. G. Dysfunction, Report of the ISHLT Working Group on Primary Lung Graft Dysfunction part II: definition. A consensus statement of the International Society for Heart and Lung Transplantation. J Heart Lung Transplant 24, 1454-1459 (2005).

3. A. Bharat, E. Kuo, N. Steward, A. Aloush, R. Hachem, E. P. Trulock, G. A. Patterson, B. F. Meyers, T. Mohanakumar, Immunological link between primary graft dysfunction and chronic lung allograft rejection. Ann Thorac Surg 86, 189-195; discussion 196-187 (2008).

4. S. A. Daud, R. D. Yusen, B. F. Meyers, M. M. Chakinala, M. J. Walter, A. A. Aloush, G. A. Patterson, E. P. Trulock, R. R. Hachem, Impact of immediate primary lung allograft dysfunction on bronchiolitis obliterans syndrome. Am J Respir Crit Care Med 175, 507-513 (2007).

5. J. A. Belperio, M. P. Keane, M. D. Burdick, V. Londhe, Y. Y. Xue, K. Li, R. J. Phillips, R. M. Strieter, Critical role for CXCR2 and CXCR2 ligands during the pathogenesis of ventilator-induced lung injury. J Clin Invest 110, 1703-1716 (2002).

6. K. Ley, C. Laudanna, M. I. Cybulsky, S. Nourshargh, Getting to the site of inflammation: the leukocyte adhesion cascade updated. Nat Rev Immunol 7, 678-689 (2007).

7. K. S. Tsai, M. H. Grayson, Pulmonary defense mechanisms against pneumonia and sepsis. Curr Opin Pulm Med 14, 260-265 (2008).

8. M. D. Tate, Y. M. Deng, J. E. Jones, G. P. Anderson, A. G. Brooks, P. C. Reading, Neutrophils ameliorate lung injury and the development of severe disease during influenza infection. J Immunol 183, 7441-7450 (2009).

9. J. A. Belperio, M. P. Keane, M. D. Burdick, B. N. Gomperts, Y. Y. Xue, K. Hong, J. Mestas, D. Zisman, A. Ardehali, R. Saggar, J. P. Lynch, 3rd, D. J. Ross, R. M. Strieter, CXCR2/CXCR2 ligand biology during lung transplant ischemia-reperfusion injury. J Immunol 175, 6931-6939 (2005).

10. A. Zarbock, M. Allegretti, K. Ley, Therapeutic inhibition of CXCR2 by Reparixin attenuates acute lung injury in mice. Br J Pharmacol 155, 357-364 (2008).

11. D. Kreisel, R. G. Nava, W. Li, B. H. Zinselmeyer, B. Wang, J. Lai, R. Pless, A. E. Gelman, A. S. Krupnick, M. J. Miller, In vivo two-photon imaging reveals monocyte-dependent neutrophil extravasation during pulmonary inflammation. Proc Natl Acad Sci U S A 107, 18073-18078 (2010).

12. O. E. Sorensen, N. Borregaard, Neutrophil extracellular traps—the dark side of neutrophils. J Clin Invest 126, 1612-1620 (2016).

13. L. Ziegler-Heitbrock, P. Ancuta, S. Crowe, M. Dalod, V. Grau, D. N. Hart, P. J. Leenen, Y. J. Liu, G. MacPherson, G. J. Randolph, J. Scherberich, J. Schmitz, K. Shortman, S. Sozzani, H. Strobl, M. Zembala, J. M. Austyn, M. B. Lutz, Nomenclature of monocytes and dendritic cells in blood. Blood 116, e74-80 (2010).

14. S. Chiu, A. Bharat, Role of monocytes and macrophages in regulating immune response following lung transplantation. Curr Opin Organ Transplant 21, 239-245 (2016).

15. R. N. Hanna, C. Cekic, D. Sag, R. Tacke, G. D. Thomas, H. Nowyhed, E. Herrley, N. Rasquinha, S. McArdle, R. Wu, E. Peluso, D. Metzger, H. Ichinose, I. Shaked, G. Chodaczek, S. K. Biswas, C. C. Hedrick, Patrolling monocytes control tumor metastasis to the lung. Science 350, 985-990 (2015).

16. J. Cros, N. Cagnard, K. Woollard, N. Patey, S. Y. Zhang, B. Senechal, A. Puel, S. K. Biswas, D. Moshous, C. Picard, J. P. Jais, D. D'Cruz, J. L. Casanova, C. Trouillet, F. Geissmann, Human CD14dim monocytes patrol and sense nucleic acids and viruses via TLR7 and TLR8 receptors. Immunity 33, 375-386 (2010).

17. A. V. Misharin, C. M. Cuda, R. Saber, J. D. Turner, A. K. Gierut, G. K. Haines, 3rd, S. Berdnikovs, A. Filer, A. R. Clark, C. D. Buckley, G. M. Mutlu, G. R. Budinger, H. Perlman, Nonclassical Ly6C(−) monocytes drive the development of inflammatory arthritis in mice. Cell Rep 9, 591-604 (2014).

18. L. M. Carlin, E. G. Stamatiades, C. Auffray, R. N. Hanna, L. Glover, G. Vizcay-Barrena, C. C. Hedrick, H. T. Cook, S. Diebold, F. Geissmann, Nr4a1-dependent Ly6C(low) monocytes monitor endothelial cells and orchestrate their disposal. Cell 153, 362-375 (2013).

19. J. L. Collison, L. M. Carlin, M. Eichmann, F. Geissmann, M. Peakman, Heterogeneity in the Locomotory Behavior of Human Monocyte Subsets over Human Vascular Endothelium In Vitro. J Immunol 195, 1162-1170 (2015).

20. C. Auffray, D. Fogg, M. Garfa, G. Elain, O. Join-Lambert, S. Kayal, S. Sarnacki, A. Cumano, G. Lauvau, F. Geissmann, Monitoring of blood vessels and tissues by a population of monocytes with patrolling behavior. Science 317, 666-670 (2007).

21. M. Nahrendorf, F. K. Swirski, E. Aikawa, L. Stangenberg, T. Wurdinger, J. L. Figueiredo, P. Libby, R. Weissleder, M. J. Pittet, The healing myocardium sequentially mobilizes two monocyte subsets with divergent and complementary functions. J Exp Med 204, 3037-3047 (2007).

22. A. Bharat, S. M. Bhorade, L. Morales-Nebreda, A. C. McQuattie-Pimentel, S. Soberanes, K. Ridge, M. M. DeCamp, K. K. Mestan, H. Perlman, G. R. Budinger, A. V. Misharin, Flow Cytometry Reveals Similarities Between Lung Macrophages in Humans and Mice. Am J Respir Cell Mol Biol 54, 147-149 (2016).

23. A. N. Desch, S. L. Gibbings, R. Goyal, R. Kolde, J. Bednarek, T. Bruno, J. E. Slansky, J. Jacobelli, R. Mason, Y. Ito, E. Messier, G. J. Randolph, M. Prabagar, S. M. Atif, E. Segura, R. J. Xavier, D. L. Bratton, W. J. Janssen, P. M. Henson, C. V. Jakubzick, Flow Cytometric Analysis of Mononuclear Phagocytes in Nondiseased Human Lung and Lung-Draining Lymph Nodes. Am J Respir Crit Care Med 193, 614-626 (2016).

24. N. Van Rooijen, The liposome-mediated macrophage ‘suicide’ technique. Journal of immunological methods 124, 1-6 (1989).

25. N. Van Rooijen, A. Sanders, Liposome mediated depletion of macrophages: mechanism of action, preparation of liposomes and applications. J Immunol Methods 174, 83-93 (1994).

26. D. Kreisel, S. Sugimoto, J. Tietjens, J. Zhu, S. Yamamoto, A. S. Krupnick, R. J. Carmody, A. E. Gelman, Bc13 prevents acute inflammatory lung injury in mice by restraining emergency granulopoiesis. J Clin Invest 121, 265-276 (2011).

27. A. Bharat, S. Chiu, Z. Zheng, H. Sun, A. Yeldandi, M. M. DeCamp, H. Perlman, G. R. Budinger, T. Mohanakumar, Lung-Restricted Antibodies Mediate Primary Graft Dysfunction and Prevent Allotolerance after Murine Lung Transplantation. Am J Respir Cell Mol Biol 55, 532-541 (2016).

28. M. Okazaki, A. S. Krupnick, C. G. Kornfeld, J. M. Lai, J. H. Ritter, S. B. Richardson, H. J. Huang, N. A. Das, G. A. Patterson, A. E. Gelman, D. Kreisel, A mouse model of orthotopic vascularized aerated lung transplantation. Am J Transplant 7, 1672-1679 (2007).

29. A. S. Krupnick, X. Lin, W. Li, R. Higashikubo, B. H. Zinselmeyer, H. Hartzler, K. Toth, J. H. Ritter, M. Y. Berezin, S. T. Wang, M. J. Miller, A. E. Gelman, D. Kreisel, Central memory CD8+ T lymphocytes mediate lung allograft acceptance. J Clin Invest 124, 1130-1143 (2014).

30. D. Kreisel, S. Sugimoto, J. Zhu, R. Nava, W. Li, M. Okazaki, S. Yamamoto, M. Ibrahim, H. J. Huang, K. A. Toth, J. H. Ritter, A. S. Krupnick, M. J. Miller, A. E. Gelman, Emergency granulopoiesis promotes neutrophil-dendritic cell encounters that prevent mouse lung allograft acceptance. Blood 118, 6172-6182 (2011).

31. A. M. Fong, L. A. Robinson, D. A. Steeber, T. F. Tedder, O. Yoshie, T. Imai, D. D. Patel, Fractalkine and CX3CR1 mediate a novel mechanism of leukocyte capture, firm adhesion, and activation under physiologic flow. The Journal of experimental medicine 188, 1413-1419 (1998).

32. R. N. Hanna, L. M. Carlin, H. G. Hubbeling, D. Nackiewicz, A. M. Green, J. A. Punt, F. Geissmann, C. C. Hedrick, The transcription factor NR4A1 (Nur77) controls bone marrow differentiation and the survival of Ly6C-monocytes. Nat Immunol 12, 778-785 (2011).

33. G. A. Rodan, H. A. Fleisch, Bisphosphonates: mechanisms of action. J Clin Invest 97, 2692-2696 (1996).

34. K. G. Anderson, K. Mayer-Barber, H. Sung, L. Beura, B. R. James, J. J. Taylor, L. Qunaj, T. S. Griffith, V. Vezys, D. L. Barber, D. Masopust, Intravascular staining for discrimination of vascular and tissue leukocytes. Nat Protoc 9, 209-222 (2014).

35. B. V. Patel, K. C. Tatham, M. R. Wilson, K. P. O'Dea, M. Takata, In vivo compartmental analysis of leukocytes in mouse lungs. Am J Physiol Lung Cell Mol Physiol 309, L639-652 (2015).

36. L. Feng, Y. Xia, T. Yoshimura, C. B. Wilson, Modulation of neutrophil influx in glomerulonephritis in the rat with anti-macrophage inflammatory protein-2 (MIP-2) antibody. J Clin Invest 95, 1009-1017 (1995).

37. J. Reutershan, M. A. Morris, T. L. Burcin, D. F. Smith, D. Chang, M. S. Saprito, K. Ley, Critical role of endothelial CXCR2 in LPS-induced neutrophil migration into the lung. J Clin Invest 116, 695-702 (2006).

38. J. H. Spahn, W. Li, A. C. Bribriesco, J. Liu, H. Shen, A. Ibricevic, J. H. Pan, B. H. Zinselmeyer, S. L. Brody, D. R. Goldstein, A. S. Krupnick, A. E. Gelman, M. J. Miller, D. Kreisel, DAP12 expression in lung macrophages mediates ischemia/reperfusion injury by promoting neutrophil extravasation. J Immunol 194, 4039-4048 (2015).

39. C. G. McKenzie, M. Kim, T. K. Singh, Y. Milev, J. Freedman, J. W. Semple, Peripheral blood monocyte-derived chemokine blockade prevents murine transfusion-related acute lung injury (TRALI). Blood 123, 3496-3503 (2014).

40. S. A. Jones, B. Dewald, I. Clark-Lewis, M. Baggiolini, Chemokine antagonists that discriminate between interleukin-8 receptors. Selective blockers of CXCR2. J Biol Chem 272, 16166-16169 (1997).

41. F. Casilli, A. Bianchini, I. Gloaguen, L. Biordi, E. Alesse, C. Festuccia, B. Cavalieri, R. Strippoli, M. N. Cervellera, R. Di Bitondo, E. Ferretti, F. Mainiero, C. Bizzarri, F. Colotta, R. Bertini, Inhibition of interleukin-8 (CXCL8/IL-8) responses by repertaxin, a new inhibitor of the chemokine receptors CXCR1 and CXCR2. Biochem Pharmacol 69, 385-394 (2005).

42. G. Thomas, R. Tacke, C. C. Hedrick, R. N. Hanna, Nonclassical patrolling monocyte function in the vasculature. Arterioscler Thromb Vasc Biol 35, 1306-1316 (2015).

43. F. Ginhoux, S. Jung, Monocytes and macrophages: developmental pathways and tissue homeostasis. Nat Rev Immunol 14, 392-404 (2014).

44. C. Sunderkotter, T. Nikolic, M. J. Dillon, N. Van Rooijen, M. Stehling, D. A. Drevets, P. J. Leenen, Subpopulations of mouse blood monocytes differ in maturation stage and inflammatory response. J Immunol 172, 4410-4417 (2004).

45. S. Jung, J. Aliberti, P. Graemmel, M. J. Sunshine, G. W. Kreutzberg, A. Sher, D. R. Littman, Analysis of fractalkine receptor CX(3)CR1 function by targeted deletion and green fluorescent protein reporter gene insertion. Molecular and cellular biology 20, 4106-4114 (2000).

46. S. Yona, K. W. Kim, Y. Wolf, A. Mildner, D. Varol, M. Breker, D. Strauss-Ayali, S. Viukov, M. Guilliams, A. Misharin, D. A. Hume, H. Perlman, B. Malissen, E. Zelzer, S. Jung, Fate mapping reveals origins and dynamics of monocytes and tissue macrophages under homeostasis. Immunity 38, 79-91 (2013).

47. J. F. Bazan, K. B. Bacon, G. Hardiman, W. Wang, K. Soo, D. Rossi, D. R. Greaves, A. Zlotnik, T. J. Schall, A new class of membrane-bound chemokine with a CX3C motif. Nature 385, 640-644 (1997).

48. D. J. Oh, B. Dursun, Z. He, L. Lu, T. S. Hoke, D. Ljubanovic, S. Faubel, C. L. Edelstein, Fractalkine receptor (CX3CR1) inhibition is protective against ischemic acute renal failure in mice. Am J Physiol Renal Physiol 294, F264-271 (2008).

49. K. Dhaliwal, E. Scholefield, D. Ferenbach, M. Gibbons, R. Duffin, D. A. Dorward, A. C. Morris, D. Humphries, A. MacKinnon, T. S. Wilkinson, W. A. Wallace, N. van Rooijen, M. Mack, A. G. Rossi, D. J. Davidson, N. Hirani, J. Hughes, C. Haslett, A. J. Simpson, Monocytes control second-phase neutrophil emigration in established lipopolysaccharide-induced murine lung injury. Am J Respir Crit Care Med 186, 514-524 (2012).

50. L. M. Carlin, C. Auffray, F. Geissmann, Measuring intravascular migration of mouse Ly6C(low) monocytes in vivo using intravital microscopy. Curr Protoc Immunol Chapter 14, Unit 14 33 11-16 (2013).

51. C. A. Leifer, A. E. Medvedev, Molecular mechanisms of regulation of Toll-like receptor signaling. J Leukoc Biol 100, 927-941 (2016).

52. W. A. Altemeier, W. C. Liles, A. Villagra-Garcia, G. Matute-Bello, R. W. Glenny, Ischemia-reperfusion lung injury is attenuated in MyD88-deficient mice. PLoS One 8, e77123 (2013).

53. G. Zanotti, M. Casiraghi, J. B. Abano, J. R. Tatreau, M. Sevala, H. Berlin, S. Smyth, W. K. Funkhouser, K. Burridge, S. H. Randell, T. M. Egan, Novel critical role of Toll-like receptor 4 in lung ischemia-reperfusion injury and edema. Am J Physiol Lung Cell Mol Physiol 297, L52-63 (2009).

54. J. B. Orens, A. Boehler, M. de Perrot, M. Estenne, A. R. Glanville, S. Keshavjee, R. Kotloff, J. Morton, S. M. Studer, D. Van Raemdonck, T. Waddel, G. I. Snell, I. S. f. H. Pulmonary Council, T. Lung, A review of lung transplant donor acceptability criteria. J Heart Lung Transplant 22, 1183-1200 (2003).

55. M. K. Pasque, Standardizing thoracic organ procurement for transplantation. J Thorac Cardiovasc Surg 139, 13-17 (2010).

56. D. K. Nayak, F. Zhou, M. Xu, J. Huang, M. Tsuji, R. Hachem, T. Mohanakumar, Long-Term Persistence of Donor Alveolar Macrophages in Human Lung Transplant Recipients That Influences Donor-Specific Immune Responses. Am J Transplant 16, 2300-2311 (2016).

57. Z. Zheng, J. Wang, X. Huang, K. Jiang, J. Nie, X. Qiao, J. Li, Improvements of the surgical technique on the established mouse model of orthotopic single lung transplantation. PLoS One 8, e81000 (2013).

58. A. V. Misharin, L. Morales-Nebreda, G. M. Mutlu, G. R. Budinger, H. Perlman, Flow cytometric analysis of macrophages and dendritic cell subsets in the mouse lung. Am J Respir Cell Mol Biol 49, 503-510 (2013).

59. E. Eden, R. Navon, I. Steinfeld, D. Lipson, Z. Yakhini, GOrilla: a tool for discovery and visualization of enriched GO terms in ranked gene lists. BMC Bioinformatics 10, 48 (2009).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method comprising treating a donor prior to harvesting an allograft from the donor with a pharmaceutical agent that inhibits the activity of non-classical monocytes.

2. The method of claim 1, wherein the agent induces apoptosis of the non-classical monocytes.

3. The method of claim 2, wherein the agent is loaded in liposomes.

4. The method of claim 1, wherein the agent is clodronate.

5. The method of claim 1, wherein the agent is a cytotoxic antibody that is targeted to the non-classical monocytes.

6. The method of claim 5, wherein the cytotoxic antibody is targeted to the cell surface protein 6-sulfo LacNAc (SLAN) on the non-classical monocytes.

7. The method of claim 1, wherein the agent is an antibody that suppresses the function of non-classical monocytes.

8. The method of claim 7, wherein the antibody binds to the cell surface receptor CS3CR1.

9. The method of claim 1 further comprising harvesting the allograft and storing the allograft in a preservation solution.

10. The method of claim 9, wherein the preservation solution comprises the pharmaceutical agent.

11. The method of claim 9, wherein the preservation solution comprises a chelator of divalent cations.

12. The method of claim 11, wherein the chelator is selected from EDTA and EGTA.

13. The method of claim 1, wherein the allograft comprises an organ or part of an organ selected from the group consisting of lung, kidney, pancreas, intestine, and liver.

14. A method comprising treating an allograft prior to transplantation with an agent that inhibits the activity of non-classical monocytes and/or an agent that depletes the allograft of non-classical monocytes.

15. The method of claim 14, wherein the agent induces apoptosis of the non-classical monocytes.

16. The method of claim 15, wherein the agent is a cytotoxic antibody that is targeted to the non-classical monocytes.

17. The method of claim 16, wherein the cytotoxic antibody is targeted to the cell surface protein 6-sulfo LacNAc (SLAN) on the non-classical monocytes.

18. The method of claim 14, wherein the agent is an antibody that suppresses the function of non-classical monocytes.

19. A method for preserving an allograft after harvest, the method comprising storing the allograft in a preservation solution comprising an agent inhibits the activity of non-classical monocytes and/or a chelator of divalent cations.

20. A composition comprising:
(a) a harvested allograft; and
(b) at least one of (i) an agent inhibits the activity of non-classical monocytes; and (ii) a chelator of divalent cations.
